SEQUENCE LISTING (12 sequences)

SEQ ID NO: 1 - DNA, 20 bp, Artificial Sequence
  DNA Primer
tttgtgaaccagtggcaggt

SEQ ID NO: 2 - DNA, 24 bp, Artificial Sequence
  DNA Primer
agtgtggatcccaagcaataccca

SEQ ID NO: 3 - DNA, 20 bp, Artificial Sequence
  DNA Primer
ccaatttccaatgctctcct

SEQ ID NO: 4 - DNA, 21 bp, Artificial Sequence
  DNA Primer
acaaccagttcgccatggtat

SEQ ID NO: 5 - DNA, 20 bp, Artificial Sequence
  DNA Primer
ggcgacctggaagtccaact

SEQ ID NO: 6 - DNA, 24 bp, Artificial Sequence
  DNA Primer
ttccgaattcactggagcctcgaa

SEQ ID NO: 7 - DNA, 20 bp, Artificial Sequence
  DNA Primer
tctccgatgcactttcgctt

SEQ ID NO: 8 - DNA, 24 bp, Artificial Sequence
  DNA Primer
tgtcctgaccactgttgtttccca

SEQ ID NO: 9 - DNA, 20 bp, Artificial Sequence
  DNA Primer
accacagtgaggaatgtcca

SEQ ID NO: 10 - DNA, 20 bp, Artificial Sequence
  DNA Primer
aagcgggtgaaacgttcctt

SEQ ID NO: 11 - DNA, 20 bp, Artificial Sequence
  DNA Primer
ccatcagcaccacagccttc

SEQ ID NO: 12 - DNA, 24 bp, Artificial Sequence
  DNA Primer
tgcacctcagggaagaatctggaa

(19) United States
(12) Patent Application Publication
(10) Pub. No.: US 2023/0357261 A1
(43) Pub. Date: Nov. 9, 2023

(54) ARYL HYDROCARBON RECEPTOR LIGANDS AND THEIR ANALOGUES FOR THE PREVENTION AND TREATMENT OF INFLAMMATORY DISORDERS

(71) Applicants: The Johns Hopkins University, Baltimore, MD (US); THE UNIVERSITY OF PITTSBURGH-OF THE COMMONWEALTH SYSTEM OF HIGHER EDUCATION, Pittsburgh, PA (US)

(72) Inventors: David Hackam, Baltimore, MD (US); Peng Lu, Baltimore, MD (US); Chhinder P. Sodhi, Baltimore, MD (US); Peter Wipf, Pittsburgh, PA (US); David Kornfilt, Pittsburgh, PA (US)

(21) Appl. No.: 18/025,797

(22) PCT Filed: Sep. 17, 2021
(86) PCT No.: PCT/US2021/050912

§ 371 Date: Mar. 10, 2023

Related U.S. Application Data
(60) Provisional application No. 63/079,749, filed on Sep. 17, 2020.

(51) Int. Cl. C07D 495/04; C07D 403/12; C07D 403/06; C07D 403/08; C07D 403/14; C07D 401/12; A61P 29/00; A23L 33/00; A23L 33/10

(52) U.S. Cl. CPC C07D 495/04; C07D 403/12; C07D 403/06; C07D 403/08; C07D 403/14; C07D 401/12; A61P 29/00; A23L 33/40; A23L 33/10

(57) ABSTRACT

Aryl hydrocarbon receptor (AHR) agonists and their use in treating or preventing or reducing the risk of an inflammatory disorder associated with a reduced expression of an aryl hydrocarbon receptor (AHR), including necrotizing enterocolitis, for preventing, reducing the risk of, or reducing the severity of an inflammatory disorder associated with a reduced expression of an aryl hydrocarbon receptor (AHR), and as an additive to infant nutritional formulas.

### FEDERALLY SPONSORED RESEARCH OR DEVELOPMENT

This invention was made with government support under DK117186 awarded by the National Institutes of Health. The government has certain rights in the invention.

### BACKGROUND

Necrotizing enterocolitis (NEC) is a serious disease of premature infants that is characterized by the acute onset of inflammation and necrosis of the intestine, leading eventually to overwhelming sepsis and death. More than 5,000 babies in the U.S. alone are afflicted with NEC and more than 50,000 premature babies born each year in the U.S. are at risk of developing NEC. The cost to the U.S. health care system to care for this population is approximately $2 billion per year. There is currently, however, no effective treatment for NEC and no effective preventive strategy.

NEC develops in the setting of prematurity, formula feeds and bacterial colonization of the newborn gastrointestinal tract, and over a third of affected patients die from the disease. In seeking to understand the pathogenesis of NEC, it has been shown that exaggerated signaling of the receptor for bacterial lipopolysaccharide, namely Toll-like receptor 4 (TLR4), on the intestinal epithelium plays a critical role in NEC development. TLR4 is expressed at higher levels in the premature as compared with the full-term intestinal epithelium, and its activation by luminal bacteria leads to mucosal death and bacterial translocation.

### SUMMARY

In some aspects, the presently disclosed subject matter provides an aryl hydrocarbon receptor (AHR) agonist comprising a compound of formula (I):

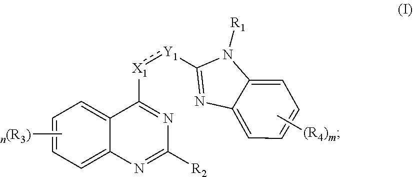

wherein:
[symbol] indicates a single or a double bond;
n and m are each independently selected from the group consisting of 0, 1, 2, 3, and 4;
X₁ is selected from the group consisting of S, O, S(═O), S(═O)₂, and —C(Rₓ)—;
Y₁ is —CH₂— or —C(R_{y})—;
wherein Rₓ and R_{y} are each independently selected from the group consisting of H, hydroxyl, cyano, and amino, or Rₓ and R_{y} together form part of a 5- to 6-membered substituted or unsubstituted cycloalkyl, cycloalkenyl, cycloheteroalkyl, cycloheteroalkenyl, aryl, or heteroaryl;
R₁ is selected from the group consisting of H, substituted or unsubstituted straightchain or branched C₁-C₆ alkyl, and —(CH₂)ₚ-Ar, wherein Ar is selected from the group consisting of substituted aryl, unsubstituted aryl, substituted heteroaryl, and unsubstituted heteroaryl, p is an integer selected from the group consisting of 0, 1, 2, 3, and 4;
R₂ is selected from the group consisting of H, substituted or unsubstituted straightchain or branched C₁-C₆ alkyl, hydroxyl, C₁-C₆ alkoxyl, halogen, —CF₃, cyano, amino, carboxyl, nitro, and mercapto;
each R₃ and R₄ are independently selected from the group consisting of H, substituted or unsubstituted straightchain or branched C₁-C₆ alkyl, hydroxyl, C₁-C₆ alkoxyl, halogen, —CF₃, cyano, amino, carboxyl, nitro, and mercapto; and
pharmaceutically acceptable salts thereof.

In certain aspects, X₁ is S or 0.

In certain aspects, R₁ is selected from the group consisting of H, substituted or unsubstituted straightchain or branched C₁-C₆ alkyl, and —(CH₂)ₚ-Ar, wherein Ar is substituted aryl or unsubstituted aryl, and p is 1.

In certain aspects, R₂ is selected from the group consisting of H, substituted or unsubstituted straightchain or branched C₁-C₆ alkyl, hydroxyl, and —CF₃.

In particular aspects, the compound of formula (I) is selected from the group consisting of:

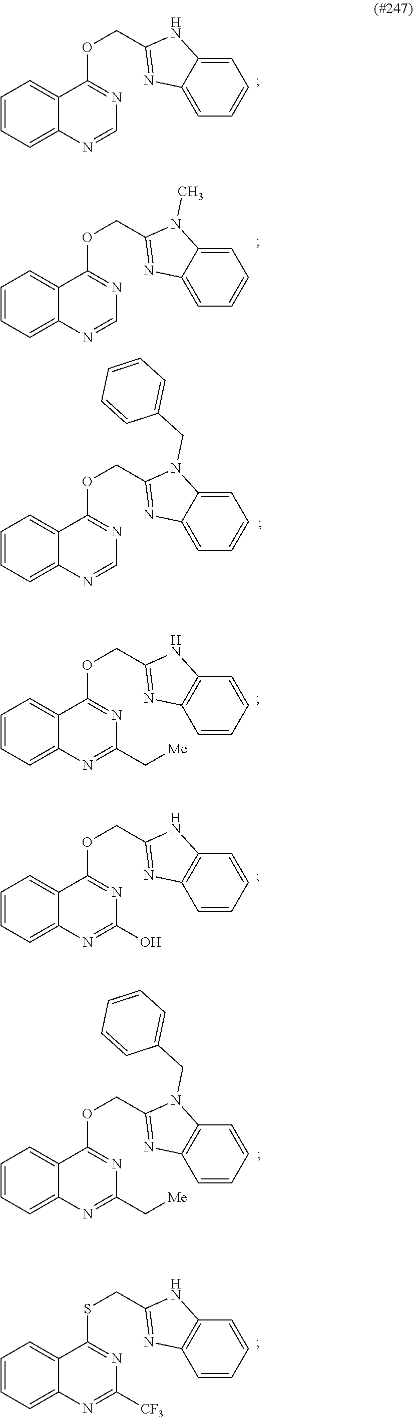

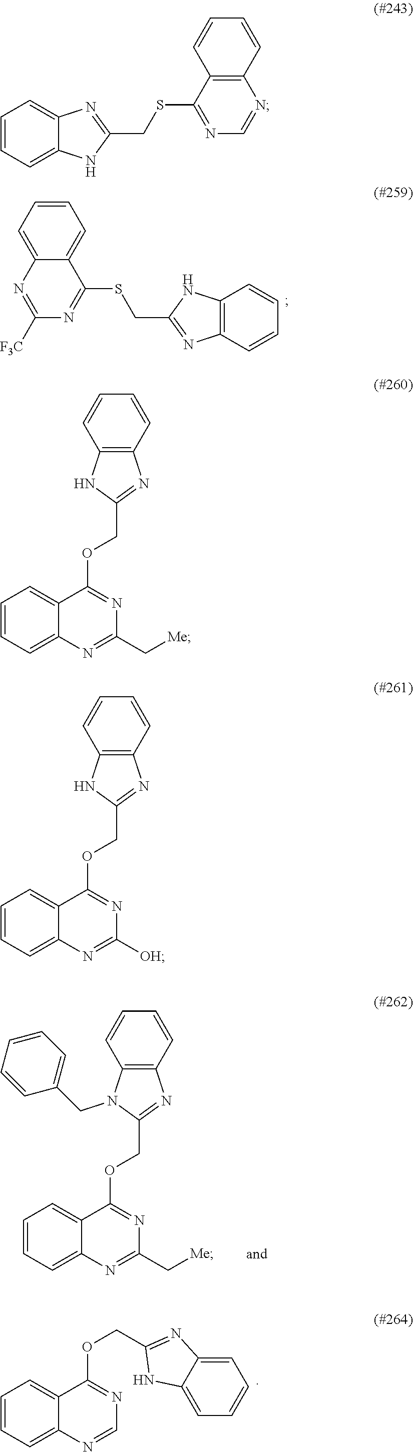

In certain aspects, X₁ is —C(Rₓ)— and Y₁ is —C(R_{y})—.

In particular aspects, the compound of formula (I) is selected from the group consisting of:

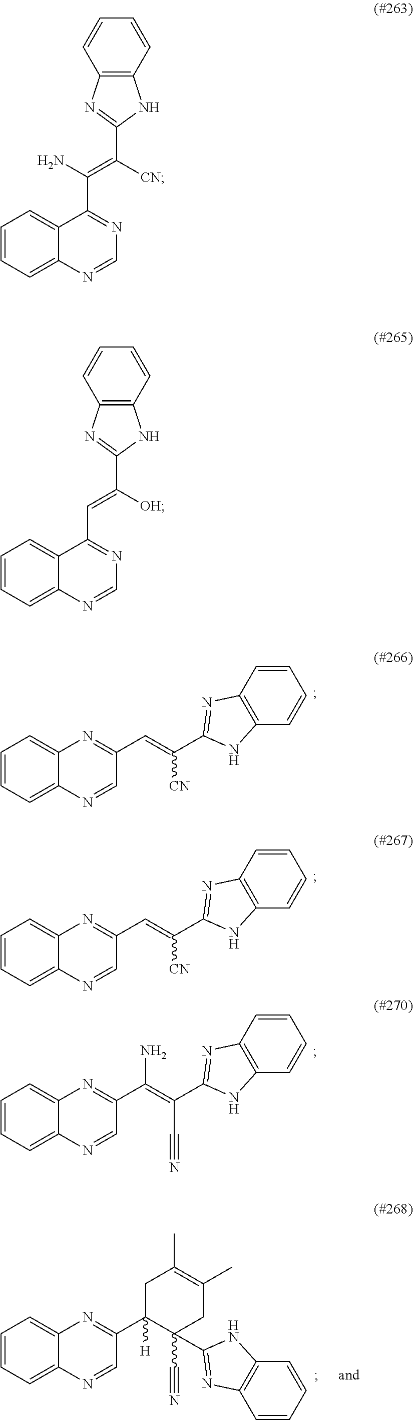 [SMILES: N#C/C(=C(/N)c1ncnc2ccccc12)c1nc2ccccc2[nH]1 ; O/C(=C\c1ncnc2ccccc12)c1nc2ccccc2[nH]1 ; N#CC(=Cc1cnc2ccccc2n1)c1nc2ccccc2[nH]1 ; N#CC(=Cc1cnc2ccccc2n1)c1nc2ccccc2[nH]1 ; N#C/C(=C(/N)c1cnc2ccccc2n1)c1nc2ccccc2[nH]1 ; CC1=C(C)CC(C#N)(c2nc3ccccc3[nH]2)C(c2cnc3ccccc3n2)C1]

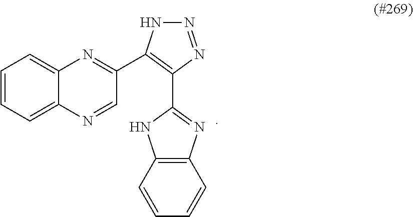 [SMILES: c1ccc2nc(-c3[nH]nnc3-c3nc4ccccc4[nH]3)cnc2c1]

In other aspects, the presently disclosed subject matter provides an aryl hydrocarbon receptor (AHR) agonist comprising a compound of formula (II):

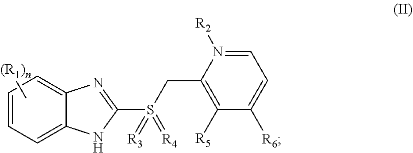

wherein:
n is an integer selected from the group consisting of 0, 1, 2, 3, and 4;
each R₁ is independently halogen or substituted or unsubstituted straightchain or branched C₁-C₆ alkyl;
R₂ is present or absent and when present is oxygen;
R₃ and R₄ are each independently present or absent and when present are oxygen;
R₅ is substituted or unsubstituted straightchain or branched C₁-C₆ alkyl;
R₆ is —O—(CH₂)ₘ-R₇, wherein R₇ is selected from the group consisting of substituted or unsubstituted straightchain or branched C₁-C₆ alkyl, —CF₃, and substituted or unsubstituted aryl; and
pharmaceutically acceptable salts thereof.

In certain aspects, the compound of formula (II) is:

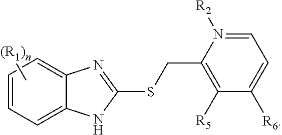

In certain aspects, the compound of formula (II) is:

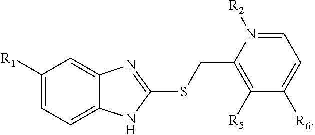

In certain aspects, the compound of formula (II) is:

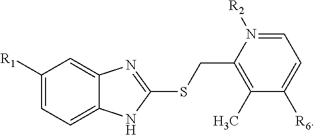

In particular aspects, the compound of formula (II) is selected from the group consisting of:

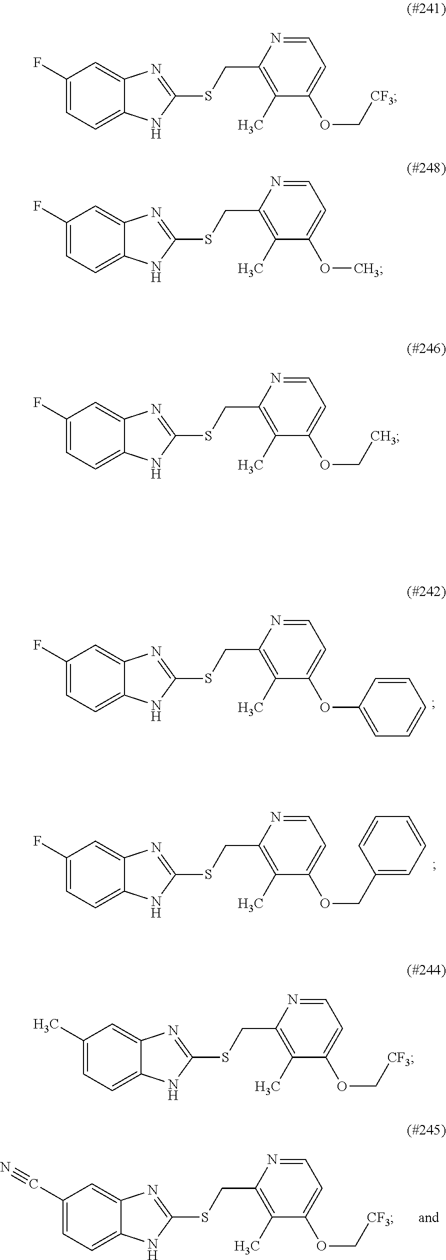

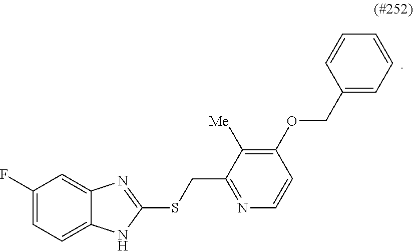 [SMILES: Cc1c(OCc2ccccc2)ccnc1CSc1nc2cc(F)ccc2[nH]1]

In certain aspects, the compound of formula (II) is:

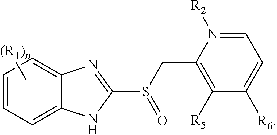

In certain aspects, the compound of formula (II) is:

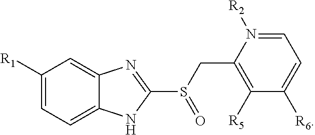

In certain aspects, the compound of formula (II) is:

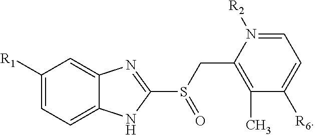

In particular aspects, the compound of formula (II) is:

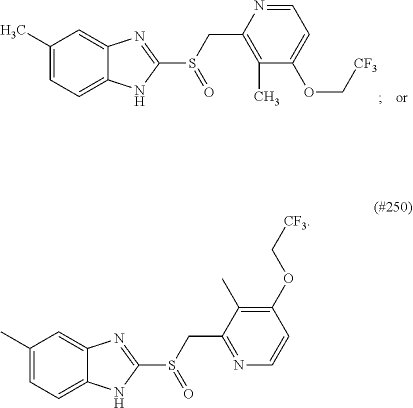 [SMILES: Cc1ccc2[nH]c(S(=O)Cc3nccc(OCC(F)(F)F)c3C)nc2c1 ; Cc1ccc2[nH]c(S(=O)Cc3nccc(OCC(F)(F)F)c3C)nc2c1]

In certain aspects, the compound of formula (II) is:

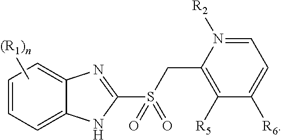

In certain aspects, the compound of formula (II) is:

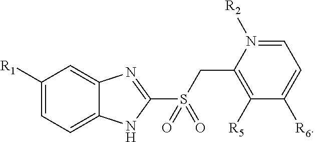

In certain aspects, the compound of formula (II) is:

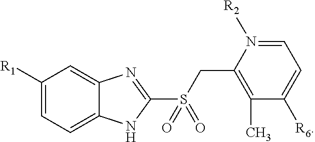

In particular aspects, the compound of formula (II) is selected from the group consisting of:

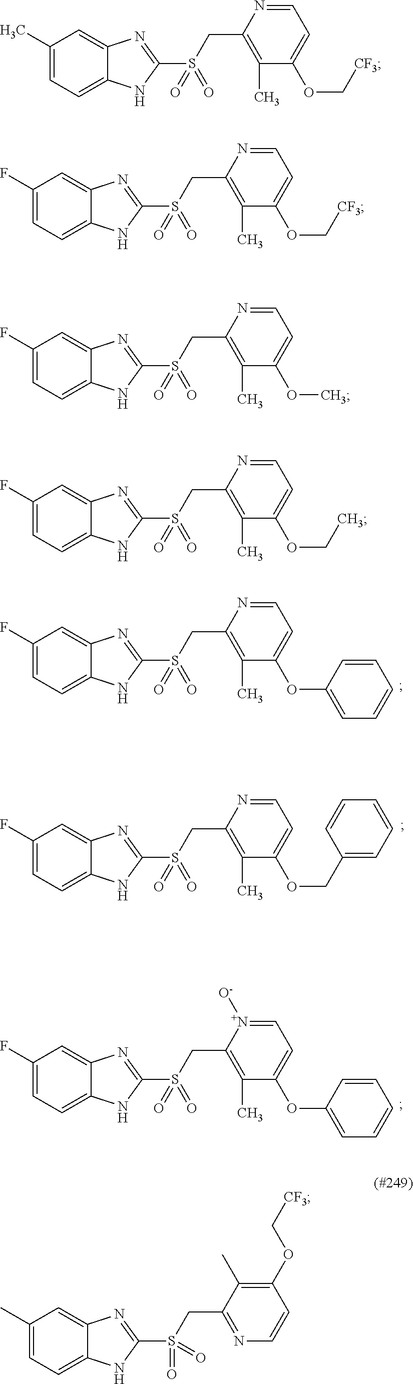

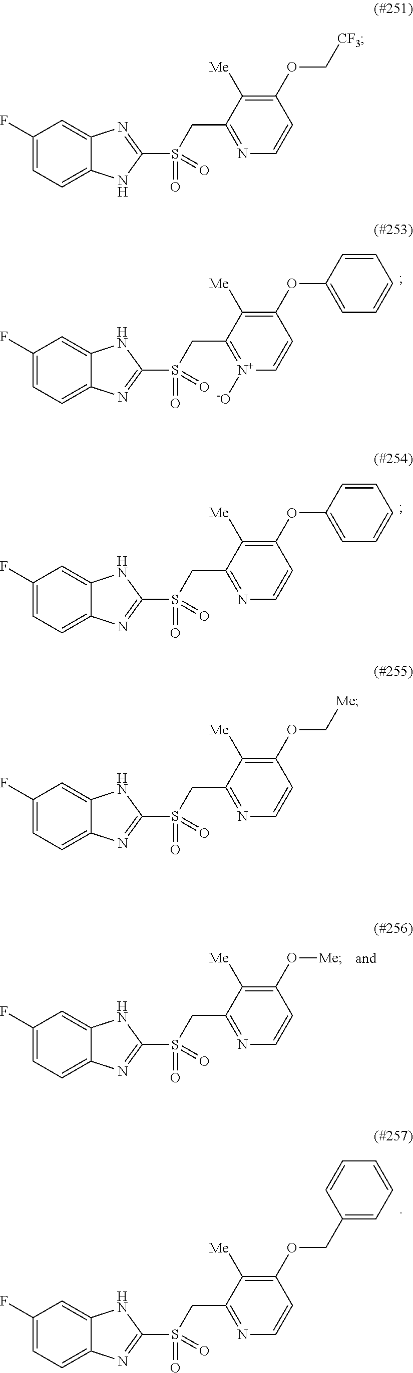

In some aspects, the presently disclosed subject matter provides an aryl hydrocarbon receptor (AHR) agonist comprising a compound of formula (III):

wherein:

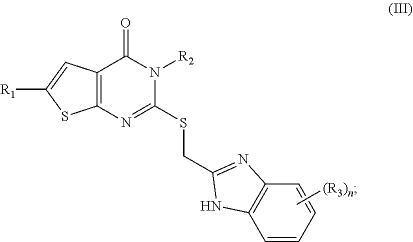

n is an integer selected from the group consisting of 0, 1, 2, 3, and 4;
R₁ is substituted or unsubstituted straightchain or branched C₁-C₆ alkyl;
R₂ is selected from the group consisting of substituted aryl, unsubstituted aryl, substituted heteroaryl, and unsubstituted heteroaryal;
each R₃ is independently selected from the group consisting of H, substituted or unsubstituted straightchain or branched C₁-C₆ alkyl, hydroxyl, C₁-C₆ alkoxyl, halogen, —CF₃, cyano, amino, carboxyl, nitro, and mercapto; and
pharmaceutically acceptable salts thereof.

In certain aspects, the compound of formula (III) is:

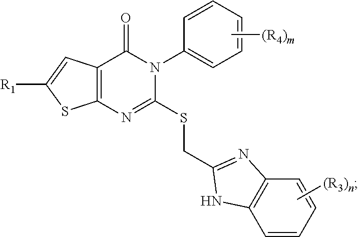

wherein:
m is an integer selected from the group consisting of 0, 1, 2, 3, 4, and 5; and
R₄ is selected from the group consisting of H, substituted or unsubstituted straightchain or branched C₁-C₆ alkyl, hydroxyl, C₁-C₆ alkoxyl, halogen, —CF₃, cyano, amino, carboxyl, nitro, and mercapto.

In particular aspects, the compound of formula (III) is:

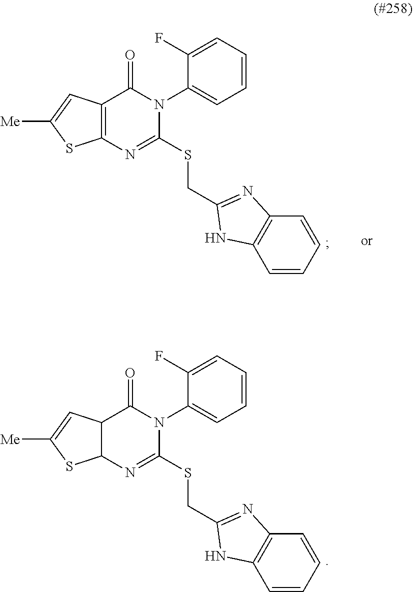 [SMILES: Cc1cc2c(=O)n(-c3ccccc3F)c(SCc3nc4ccccc4[nH]3)nc2s1 ; CC1=CC2C(=O)N(c3ccccc3F)C(SCc3nc4ccccc4[nH]3)=NC2S1]

In some aspects, the presently disclosed subject matter provides a method for treating or preventing or reducing the risk of an inflammatory disorder associated with a reduced expression of an aryl hydrocarbon receptor (AHR) in a subject in need of treatment thereof, the method comprising administering to the subject a compound of formula (I-III), a compound having the following structure:

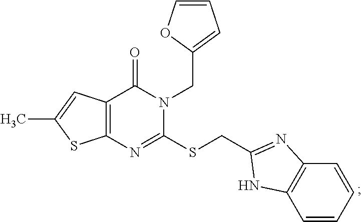 [SMILES: Cc1cc2c(=O)n(Cc3ccco3)c(SCc3nc4ccccc4[nH]3)nc2s1]

or pharmaceutically acceptable salts thereof, to activate the AHR, thereby treating or preventing or reducing the risk of the inflammatory disorder.

In particular aspects, the inflammatory disorder is necrotizing enterocolitis. In certain aspects, the subject is an infant. In more certain aspects, the subject is a premature infant.

In other aspects, the presently disclosed subject matter provides an infant nutritional formula comprising a therapeutically effective amount of one or more compounds of formula (I-III), a compound having the following structure:

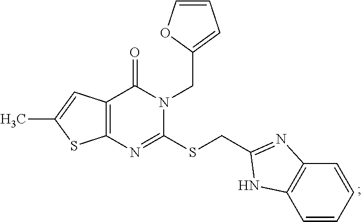 [SMILES: Cc1cc2c(=O)n(Cc3ccco3)c(SCc3nc4ccccc4[nH]3)nc2s1]

or pharmaceutically acceptable salts thereof.

In other aspects the presently disclosed subject matter provides a method for preventing, reducing the risk of, or reducing the severity of an inflammatory disorder associated with a reduced expression of an aryl hydrocarbon receptor (AHR) in a subject in need of treatment thereof, the method comprising administering to a mother while pregnant with the subject one or more compounds of formula (I-III), or a compound having the following structure:

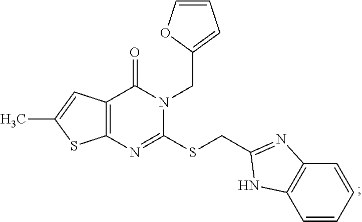 [SMILES: Cc1cc2c(=O)n(Cc3ccco3)c(SCc3nc4ccccc4[nH]3)nc2s1]

or pharmaceutically acceptable salts thereof, to activate the AHR, thereby treating or preventing, reducing the risk of, or reducing the severity of the inflammatory disorder.

In certain aspects, the mother is at risk for delivering the subject prematurely.

Certain aspects of the presently disclosed subject matter having been stated hereinabove, which are addressed in whole or in part by the presently disclosed subject matter, other aspects will become evident as the description proceeds when taken in connection with the accompanying Examples and Figures as best described herein below.

### BRIEF DESCRIPTION OF THE FIGURES

The patent or application file contains at least one drawing executed in color. Copies of this patent or patent application publication with color drawing(s) will be provided by the Office upon request and payment of the necessary fee.

Having thus described the presently disclosed subject matter in general terms, reference will now be made to the accompanying Figures, which are not necessarily drawn to scale, and wherein:

FIG. 1a, FIG. 1B, FIG. 1c, FIG. 1d, FIG. 1e, FIG. 1f, FIG. 1g, FIG. 1h, FIG. 1i, FIG. 1j, FIG. 1k, FIG. 1l, and FIG. 1m show that administration of the AHR ligand I3C during pregnancy protects against NEC in the newborn offspring. FIG. 1a, Schematic illustrating the experimental setup in which administration of the AHR ligand I3C to the pregnant mother can be evaluated for effects on the development of NEC in the pups. FIG. 1B, FIG. 1c, and FIG. 1d, mRNA expression of AHR activation marker Cyp1a1 in the ileum of pregnant mice (FIG. 1B), the fetus (FIG. 1c), and newborn mice (FIG. 1d), on control diet (Ctrl), or maternal I3C enriched diet (M-I3C, 25 mg/kg body weight/day). FIG. 1e-FIG. 1f, Representative H&E-stained micrographs of the terminal ileum in pups with NEC from mothers (i.e., dams) who were fed either a control diet or a diet rich in I3C (M-I3C, 25 mg/kg body weight/day); NEC severity (FIG. 10; FIG. 1g, qRT-PCR showing expression of 116 in the terminal ileum. FIG. 1h, confocal images of ileal sections stained with the apoptosis marker TUNEL. FIG. 1i, quantification of the fluorescent intensity in FIG. 1h using ImageJ. FIG. 1j, FIG. 1k, and FIG. 1l, AHR activation measured as Cyp1a1 expression in the intestinal epithelial cell line (IEC-6) treated with amniotic fluid (FIG. 1j), breast milk (FIG. 1k), and serum (FIG. 1l) that were harvested from the pregnant mice that were either on the control diet (Ctrl) or I3C enriched diet (M-I3C, 25 mg/kg body weight/day); FIG. 1m, qRT-PCR showing expression of 116 in the ileum induced by LPS (50 μg/mL for 6 hours) injection in neonatal mice, born from mice fed on Ctrl or maternal I3C enriched diet (M-I3C, 25 mg/kg body weight/day) during pregnancy. Scale bars in FIG. 1h, 50 μm. *p<0.05, **p<0.01, ***p<0.001, p values obtained from two-sided t-tests. Each dot in graphs represents data from an individual mouse or well;

FIG. 2a, FIG. 2b, FIG. 2c, FIG. 2d, FIG. 2e, FIG. 2f, FIG. 2g, FIG. 2h, FIG. 2i, FIG. 2j, FIG. 2k, and FIG. 2l show that AHR expression on the intestinal epithelium is required for protection against NEC. FIG. 2a-FIG. 2b, d-FIG. 2e, FIG. 2g-FIG. 2h show representative confocal images and quantification of fluorescent intensity of AHR immunostained ileal sections of human (FIG. 2a-FIG. 2b), mouse (FIG. 2d-FIG. 2e), piglet (FIG. 2g-FIG. 2h) ileal specimens from Ctrl and NEC patients or animals. FIG. 2c, FIG. 2f, FIG. 2i show expression of AHR by qRT-PCR in the small intestine of human (FIG. 2c), mouse (FIG. 2f), and piglets (FIG. 2i). FIG. 2j is H&E-stained representative images showing abnormal NEC histology in mice induced to develop experimental NEC in wildtype (WT), AHR knockout (Ahr^{-/-}), AHR intestinal epithelial cells knockout (Ahr^{ΔIEC}), and AHR myeloid knockout (Ahr^{Δlys}). FIG. 2k shows quantification of NEC severity. FIG. 2l shows qRTPCR expression of Tnf-a in the intestinal epithelium. Scale bars in FIG. 2a, FIG. 2d, FIG. 2j are 50 μm. *p<0.05, **p<0.01, ***p<0.001, p values obtained either from two-sided t-tests or one-way ANOVA followed by multiple comparisons using Prism 8 software. Each dot in graphs represents data from an individual human specimen, mouse, piglet;

FIG. 3a, FIG. 3b, FIG. 3c, and FIG. 3d shown that feeding mice the AHR ligand protects against NEC by activating AHR on the intestinal epithelium. FIG. 3a, FIG. 3d, H&E stained representative images (FIG. 3a) and the quantification of NEC severity (FIG. 3d) in ileal sections of newborn mice, supplemented with AHR ligand I3C (25 mg/kg body weight/day for 4 days) and induced to develop experimental NEC. Data showing I3C mediated protection against NEC development only in wildtype (WT) and AHR myeloid knockout (Ahr^{Δlys}) mice but not in AHR knockout (Ahr^{-/-}) and AHR intestinal epithelial cells knockout (Ahr^{ΔIEC}). FIG. 3b, dot graph showing AHR ligand I3C supplementation (25 mg/kg body weight/day for 4 days) produced a multifold induction of AHR activation marker, Cyp1a1 in the ileum of wildtype, WT but not in Ahr^{-/-}, and Ahr^{ΔIEC} and a moderate increase in Ahr^{Δlys} mice. FIG. 3c, mRNA levels of pro-inflammatory cytokine Tnf-a. *p<0.05, **p<0.01, ***p<0.001, p values obtained from two-sided t-tests. Each dot in graphs represents data from an individual mouse;

FIG. 4a, FIG. 4b, FIG. 4c, FIG. 4d, FIG. 4e, FIG. 4f, FIG. 4g, FIG. 4h, FIG. 4j, and FIG. 4k show that AHR activation limits TLR4 signaling and expression in the intestinal epithelium of mice and humans. FIG. 4a shows representative confocal images of enteroids harvested from wildtype (WT) and AHR knockout (Ahr^{-/-}) mice after 7 days of culture on Matrigel, and stained with the epithelial marker (Ecadherin, Ecad), actin filaments (Rhodamine Phalloidin, RP) and nuclei (DAPI). FIG. 4b-FIG. 4c show qRT-PCR expression of Cyp1a1 and Tnf-a in these enteroids under the indicated condition; FIG. 4d-FIG. 4f are qRT-PC showing the expression of Cyp1a1 (FIG. 4d), Tnf-a (FIG. 4e) and Tlr4 (FIG. 40 in the ileum of newborn mice after treatment with or without I3C (25 mg/kg body weight/day for 4 days). FIG. 4g-FIG. 4i, qRT-PCR showing the expression of the Tlr4 regulatory microRNAs, miR-146b (FIG. 4g), let-7i (FIG. 4h) and miR-223 (FIG. 4i) in the ileum of WT and Ahr^{-/-} mice in the presence or absence of I3C (25 mg/kg body weight/day for 24 hours). FIG. 4j-FIG. 4k are qRT-PCR showing expression of Cyp1a1 (FIG. 4j) and Tnf-a (FIG. 4k) in the presence of I3C (200 μM I3C pretreatment for 15 minutes and then additional 6 hours) and LPS (50 μg/mL for 6 hours). Scale bars in FIG. 4a, 25 μm. *p<0.05, **p<0.01, ***p<0.001, p values obtained using one-way ANOVA followed by multiple comparisons. Each dot in graphs represents data from an individual mouse or well of human explant culture;

FIG. 5a, FIG. 5b, FIG. 5c, FIG. 5d, FIG. 5e, FIG. 5f, FIG. 5g, FIG. 5h, FIG. 5i, and FIG. 5j show that breast milk activates AHR on the intestinal epithelium and protects against experimental NEC in newborn mice. FIG. 5a-FIG. 5b are qRT-PCR showing the expression of Cyp1a1 (FIG. 5a) and Tnf-a (FIG. 5b) in mouse intestinal enteroids from wildtype and Ahr^{-/-} mice, treated with saline or LPS in the presence or absence of breast milk; FIG. 5c-FIG. 5d show LPS-induced apoptosis in enteroids measured by TUNEL assay in wild-type and Ahr^{-/-} mouse enteroids as quantified (FIG. 5c) and revealed (FIG. 5d) by confocal microscopy; FIG. 5e-FIG. 5f show qRT-PCR expression of Cyp1a1 (FIG. 5 FIG. 5e) and 116 (FIG. 5f) in wildtype and Ahr^{-/-} mice treated with saline, LPS and/or breast milk as indicated. FIG. 5g-FIG. 5j, representative H&E stained section (FIG. 5g), NEC severity score (FIG. 5h) iNosS qRT-PCR (FIG. 5i), and Tlr4 qRT-PCR (FIG. 5j), in wildtype and Ahr^{-/-} mice induced to develop NEC in the presence or absence of breast milk as indicated. Scale bars in FIG. 5d, 10 μm. *p<0.05, **p<0.01, ***p<0.001, p values obtained using one-way ANOVA followed by multiple comparisons using Prism 8 software. Each dot in graphs represents data from an individual mouse or individual well of enteroids;

FIG. 6a, FIG. 6b, FIG. 6c, FIG. 6d, FIG. 6e, FIG. 6f, FIG. 6g, FIG. 6h, FIG. 6i, FIG. 6j, FIG. 6k, FIG. 6l, FIG. 6m, FIG. 6n, FIG. 6o, FIG. 6p, and FIG. 6q show that AHR agonist A18 activates AHR on the intestinal epithelium, reduces TLR4 signaling, and protects against experimental NEC in newborn mice. FIG. 6a is a dose-response curve of A18 for luciferase activity in AHR-reporter IEC-6 cells. FIG. 6b is the molecular formula of A18. FIG. 6c shows qRT-PCR expression of Cyp1a1 in IEC-6 cells treated with A18 (2004 for 6 hours). FIG. 6d-FIG. 6f shows qRT-PCR expression of Cyp1a1 (FIG. 6d), 116 induction (FIG. 6e), and Tlr4 (FIG. 60 in the ileum of wildtype (WT) and not AHR knockout (Ahr^{-/-}) mice. FIG. 6g-FIG. 6i are representative histological H&E of the terminal ileum (FIG. 6g), NEC severity score (FIG. 6h), 116 expression (FIG. 6i) in newborn mice with NEC after administration of A18 (300 mg/kg body weight/day for 4 days). FIG. 6j-FIG. 6l are qRTPCR showing expression of Cyp1a1 (FIG. 6j), Tnf-a (FIG. 6k) and Tlr4 (FIG. 6l) in freshly harvested human ileum obtained at surgery at treated with LPS (50 μg/ML for 6 hours) and/or A18 (2004 A18 pretreatment for 15 minutes and then additional 6 hours). FIG. 6m-FIG. 6q show expression Cyp1a1 in the neonatal ileum after maternal administration of A18 (FIG. 6m, 300 mg/kg body weight/day for 4 days), ileal H&E of the newborn (FIG. 6n), NEC severity (FIG. 6o), expression of Tnf-a (FIG. 6p), and Tlr4 after NEC induction in the absence or presence of maternal A18 (M-A18, 300 mg/kg body weight/day for 4 days) (FIG. 6q). *p<0.05, **p<0.01, ***p<0.001, p values obtained either from two-sided t-tests or using one-way ANOVA followed by multiple comparisons. Each dot in graphs represents data from an individual mouse or human specimen;

FIG. 7 demonstrates AHR expression on the mouse ileum. Representative confocal images of ileal sections from P11 wildtype, Ahr^{-/-}, Ahr^{ΔIEC} and Ahr^{Δlys} mice stained with AHR (green) and DAPI (blue);

FIG. 8a, FIG. 8b, FIG. 8c, and FIG. 8d demonstrate evaluation of Ahr transgenic mice treated with breast milk and I3C. FIG. 8a and FIG. 8b are representative images of H&E-stained ileal sections from breast fed and/or I3C-treated (25 mg/kg body weight/day for 4 days) newborn wild-type, Ahr^{-/-}, Ahr^{ΔIEC} and Ahr^{Δlys} mice. FIG. 8c and FIG. 8d are qRT-PCR showing expression of Tnf-a under the indicated conditions. Each dot represents data from an individual mouse; comparison by ANOVA;

FIG. 9a, FIG. 9b, FIG. 9c, FIG. 9d, FIG. 9e, FIG. 9f, FIG. 9g, FIG. 9h, and FIG. 9i show that AHR activation protects against NEC independent of IL-22 and intestinal permeability. FIG. 9a, FIG. 9b, and FIG. 9c show mRNA expression of Cyp1a1 (FIG. 9a) Tnf-a (FIG. 9b), and representative H&E-stained ileum (FIG. 9c) in wildtype and Il22^{-/-} mice subjected to experimental NEC (NEC) with or without I3C (NEC+I3C, 25 mg/kg body weight/day for 4 days). FIG. 9d, FIG. 9e, FIG. 9f, and FIG. 9g are flow cytometry showing % relative to total lamina propria lymphocytes (LPL) of Th17 cells (FIG. 9d), ILC1 cells (FIG. 9e), ILC2 cells (FIG. 90 and ILC3 cells (FIG. 9g) in the lamina propria of ileum of WT and Ahr^{-/-} mice. FIG. 9h is representative confocal images stained with ZO-1 in the ileum of WT and Ahr^{-/-} mice. FIG. 9i shows concentration of 4-kDa FITC-dextran in the serum of WT and Ahr^{-/-} mice showing the intestinal permeability. Scale bars: 50 μm. *p<0.05, **p<0.01, ***p<0.001, p values obtained from two-sided t-tests. Each dot in graphs represents data from an individual mouse;

FIG. 10 shows the chemical structures of compound #169 and representative analogues;

FIG. 11 and FIG. 12 are qRT-PCR showing the expression of Tnf-a (FIG. 11) and Cyp1a1 (FIG. 12) in mouse intestinal enteroids from wildtype mice, treated with saline or LPS in the presence or absence of 20 μM or 1 μM of I3C, A18, compound #169 (old batch, 1.5-year-old, 1.5-yr) and compound #169 (new batch, 0-year-old, 0-yr);

FIG. 13 and FIG. 14 are qRT-PCR showing the expression of Tnf-a (FIG. 13) and Cyp1a1 (FIG. 14) in mouse intestinal enteroids from wildtype mice, treated with saline or LPS in the presence or absence of 20 μM of compound #169 and its analogs (compounds #241, #242, #243, #244, #245 #246, #247 and #248);

FIG. 15a and FIG. 15b are qRT-PCR showing the expression of Cyp1a1 (FIG. 15a) and Tnf-a (FIG. 15b) in mouse intestinal enteroids from wildtype mice, treated with DMSO vehicle or LPS in the presence or absence of compound #169 and its analogs (compounds #241, #242, #243, #244, #245 #246, #247 and #248);

FIG. 16a, FIG. 16b, FIG. 16c, FIG. 16d, FIG. 16e, FIG. 16f, FIG. 16g, FIG. 16h, FIG. 16i, and FIG. 16j show that administration of compound #169, compound #243, or compound #247 didn't not reduce the severity of NEC in mouse intestine. FIG. 16a-FIG. 16e show qRT-PCR expression of Cyp1a1 (FIG. 16a), IL-1b (FIG. 16b), IL-6 (FIG. 16c), Tnf-a (FIG. 16d) and Lcn2 (FIG. 16e) in the ileum of healthy control mice (Ctrl), mice with NEC (NEC), and mice with NEC administrated with compound #169 (NEC #169), compound #243 (NEC #243), or compound #247 (NEC #247) (20 mg/kg body weight/day for 4 days). FIG. 16f-FIG. 16j, representative H&E-stained section of the ileum of healthy control mice (Ctrl), mice with NEC (NEC), and mice with NEC administrated with compound #169 (NEC #169), compound #243 (NEC #243), or compound #247 (NEC #247) (20 mg/kg body weight/day for 4 days). *p<0.05, **p<0.01, ***p<0.001, p values obtained from Kruskal-Wallis test followed by multiple comparisons using Prism 8 software. Each dot in graphs represents data from an individual mouse;

FIG. 17 shows the chemical structures of compound #169 and analogues (compound #249, #250, #251, #252, #253, #254, #255, #256, #257, #258, #259, #260, #261, #262, #263, #264, #265, #266, #267, #268, #269 and #270);

FIG. 18a, FIG. 18b, FIG. 18c, and FIG. 18d are qRT-PCR showing the expression of Cyp1a1 in mouse intestinal enteroids from wildtype mice, treated with DMSO vehicle or LPS in the presence or absence of compound #169 and its analogs (compound #249, #250, #251, #252, #253, #254, #255, #256, #257 (FIG. 18a), #258, #259, #260, #261, #262 (FIG. 18b), #263, #264, #265 (FIG. 18c), #266, #267, #268, #269 and #270 (FIG. 18d));

FIG. 19a, FIG. 19b, FIG. 19c, and FIG. 19d are qRT-PCR showing the expression of Tnf-a in mouse intestinal enteroids from wildtype mice, treated with DMSO vehicle or LPS in the presence or absence of compound #169 and its analogs (compound #249, #250, #251, #252, #253, #254, #255, #256, #257 (FIG. 19a), #258, #259, #260, #261, #262 (FIG. 19b), #263, #264, #265 (FIG. 19c), #266, #267, #268, #269 and #270 (FIG. 19d)); and

FIG. 20a, FIG. 20b, FIG. 20c, FIG. 20d, FIG. 20e, FIG. 20f, and FIG. 20g show that compound #263 protects against experimental NEC in newborn mice. FIG. 20a-FIG. 20e show qRT-PCR expression of Cyp1a1 (FIG. 20a), IL-1b (FIG. 20b), IL-6 (FIG. 20c), Tnf-a (FIG. 20d) and Lcn2 (FIG. 20e) in the ileum of mice with NEC (NEC) and mice with NEC administrated with compound #263 (NEC #263) (20 mg/kg body weight/day for 4 days). FIG. 20f-FIG. 20g, representative H&E-stained section of the ileum of mice with NEC (NEC) and mice with NEC administrated with compound #263 (NEC #263) (20 mg/kg body weight/day for 4 days). *p<0.05, **p <0.01, ***p<0.001, p values obtained from Mann-Whitney test using Prism 8 software. Each dot in graphs represents data from an individual mouse.

### DETAILED DESCRIPTION

The presently disclosed subject matter now will be described more fully hereinafter with reference to the accompanying Figures, in which some, but not all embodiments of the inventions are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Figures. Therefore, it is to be understood that the presently disclosed subject matter is not to be limited to the specific embodiments disclosed and that modifications and other embodiments are intended to be included within the scope of the appended claims.

I. Aryl Hydrocarbon Receptor Ligands and their Analogues for the Prevention and Treatment of Inflammatory Disorders

The presently disclosed subject matter demonstrates, in part, that activation of the aryl hydrocarbon receptor (AHR) in mice and human tissue can inhibit TLR4 signaling, and thus serve to dampen inflammation in the gut. The finding that AHR activation could inhibit TLR4 signaling led to a search for novel AHR agonists that could be used as therapeutics for NEC. To this end, the AHR analogue “A18”, which had been identified and sold commercially as the proton pump inhibitor “Lansoprazole,” was identified as an AHR agonists. The administration of lansoprazole, i.e., A18, effectively prevented and treated NEC in mouse models, and dampened inflammation in the intestine of human resected at the time of surgery for NEC.

Analogues of A18 were then synthesized and developed. A total of 235 analogues were tested in mouse enteroid cell lines. One compound in particular, referred to herein as compound 169, was identified that activated AHR and significantly inhibited TLR4 signaling in the gut. The current technology seeks to prevent or treat inflammatory conditions that affect children, most notably necrotizing enterocolitis (NEC), by activating the aryl hydrocarbon receptor (AHR).

Importantly, it is shown that A18 can be administered to pregnant mothers (mice) to treat NEC in the premature pups. This observation suggests that compound C169 could potentially be administered as a gut protective agent for the fetus when administered to the mother in the event of premature labor. C169 could potentially solve the problem of necrotizing enterocolitis, the leading cause of death in premature infants from gastrointestinal disease, for which there is no specific treatment and no cure. The induction of NEC is always related to the provision of infant formula, as breast milk is highly protective. There is no formula that is NEC protective. Accordingly, the presently disclosed compounds could be developed into a drug for treating NEC and/or an agent that could be added to infant formula to prevent NEC.

### A. Compounds

In some embodiments, the presently disclosed subject matter provides an aryl hydrocarbon receptor (AHR) agonist comprising a compound of formula (I):

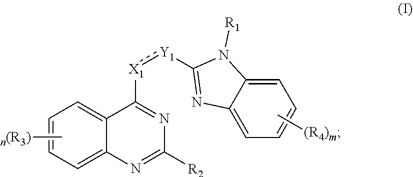

wherein:
[symbol] indicates a single or a double bond;
n and m are each independently selected from the group consisting of 0, 1, 2, 3, and 4;
X₁ is selected from the group consisting of S, O, S(═O), S(═O)₂, and —C(Rₓ)—;
Y₁ is —CH₂— or —C(R_{y})—;
wherein Rₓ and R_{y} are each independently selected from the group consisting of H, hydroxyl, cyano, and amino, or Rₓ and R_{y} together form part of a 5- to 6-membered substituted or unsubstituted cycloalkyl, cycloalkenyl, cycloheteroalkyl, cycloheteroalkenyl, aryl, or heteroaryl;
R₁ is selected from the group consisting of H, substituted or unsubstituted straightchain or branched C₁-C₆ alkyl, and —(CH₂)ₚ-Ar, wherein Ar is selected from the group consisting of substituted aryl, unsubstituted aryl, substituted heteroaryl, and unsubstituted heteroaryl, p is an integer selected from the group consisting of 0, 1, 2, 3, and 4;
R₂ is selected from the group consisting of H, substituted or unsubstituted straightchain or branched C₁-C₆ alkyl, hydroxyl, C₁-C₆ alkoxyl, halogen, —CF₃, cyano, amino, carboxyl, nitro, and mercapto;
each R₃ and R₄ are independently selected from the group consisting of H, substituted or unsubstituted straightchain or branched C₁-C₆ alkyl, hydroxyl, C₁-C₆ alkoxyl, halogen, —CF₃, cyano, amino, carboxyl, nitro, and mercapto; and
pharmaceutically acceptable salts thereof.

In certain embodiments, X₁ is S or O.

In certain embodiments, R₁ is selected from the group consisting of H, substituted or unsubstituted straightchain or branched C₁-C₆ alkyl, and —(CH₂)ₚ—Ar, wherein Ar is substituted aryl or unsubstituted aryl, and p is 1.

In certain embodiments, R₂ is selected from the group consisting of H, substituted or unsubstituted straightchain or branched C₁-C₆ alkyl, hydroxyl, and —CF₃.

In particular embodiments, the compound of formula (I) is selected from the group consisting of:

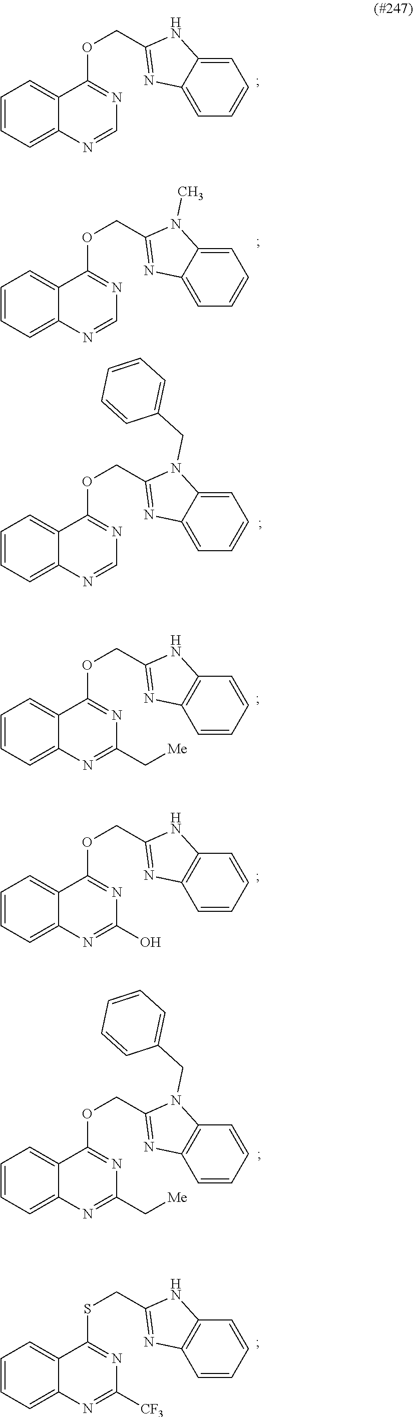

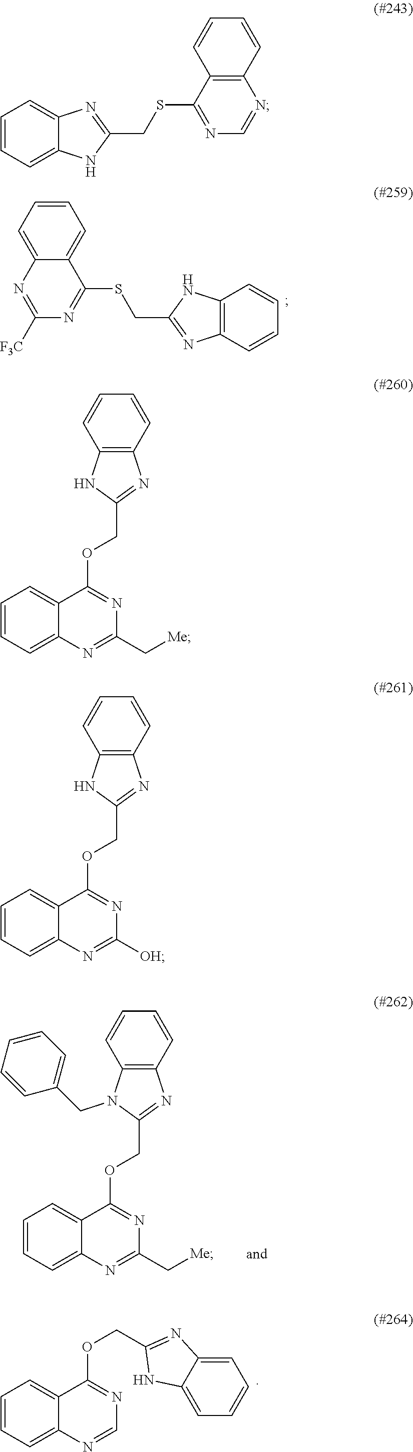

In certain embodiments, X₁ is —C(Rₓ)— and Y₁ is —C(R_{y})—.

In particular embodiments, the compound of formula (I) is selected from the group consisting of:

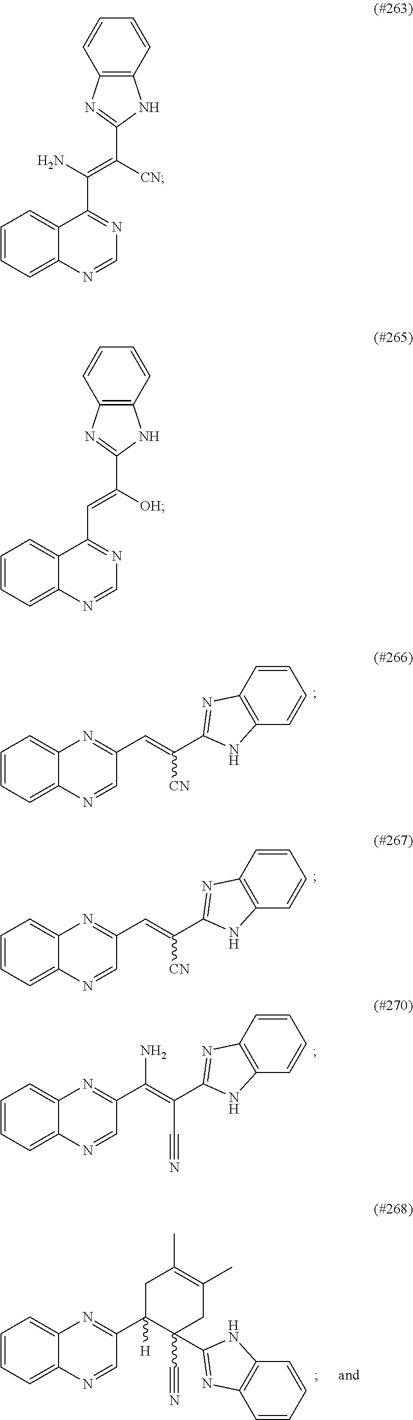 [SMILES: N#C/C(=C(/N)c1ncnc2ccccc12)c1nc2ccccc2[nH]1 ; O/C(=C\c1ncnc2ccccc12)c1nc2ccccc2[nH]1 ; N#CC(=Cc1cnc2ccccc2n1)c1nc2ccccc2[nH]1 ; N#CC(=Cc1cnc2ccccc2n1)c1nc2ccccc2[nH]1 ; N#C/C(=C(/N)c1cnc2ccccc2n1)c1nc2ccccc2[nH]1 ; CC1=C(C)CC(C#N)(c2nc3ccccc3[nH]2)C(c2cnc3ccccc3n2)C1]

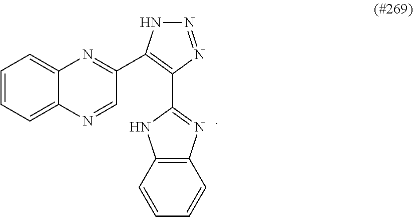 [SMILES: c1ccc2nc(-c3[nH]nnc3-c3nc4ccccc4[nH]3)cnc2c1]

In other embodiments, the presently disclosed subject matter provides an z hydrocarbon receptor (AHR) agonist comprising a compound of formula (II):

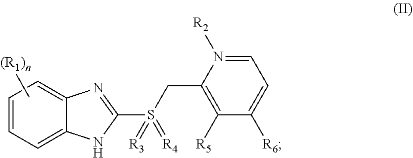

wherein:
n is an integer selected from the group consisting of 0, 1, 2, 3, and 4;
each R₁ is independently halogen or substituted or unsubstituted straightchain or branched C₁-C₆ alkyl;
R₂ is present or absent and when present is oxygen;
R₃ and R₄ are each independently present or absent and when present are oxygen;
R₅ is substituted or unsubstituted straightchain or branched C₁-C₆ alkyl;
R₆ is —O—(CH₂)ₘ-R₇, wherein R₇ is selected from the group consisting of substituted or unsubstituted straightchain or branched C₁-C₆ alkyl, —CF₃, and substituted or unsubstituted aryl; and
pharmaceutically acceptable salts thereof.

In certain embodiments, the compound of formula (II) is:

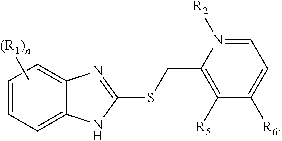

In certain embodiments, the compound of formula (II) is:

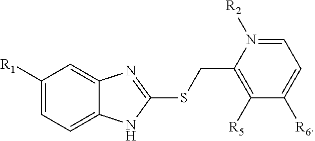

In certain embodiments, the compound of formula (II) is:

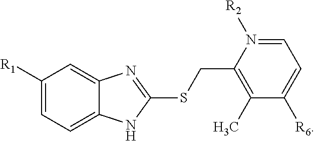

In particular embodiments, the compound of formula (II) is selected from the group consisting of:

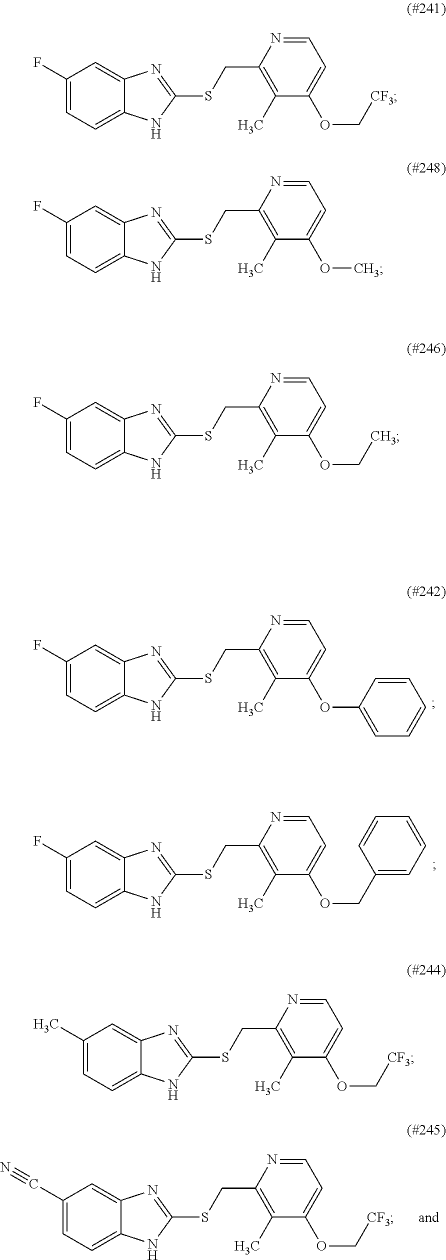

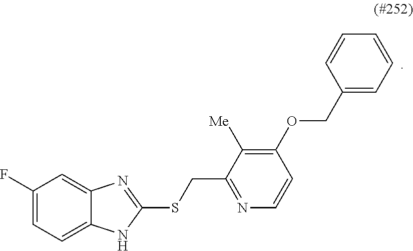 [SMILES: Cc1c(OCc2ccccc2)ccnc1CSc1nc2cc(F)ccc2[nH]1]

In certain embodiments, the compound of formula (II) is:

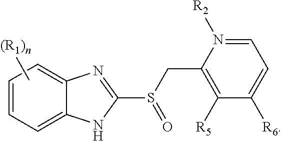

In certain embodiments, the compound of formula (II) is:

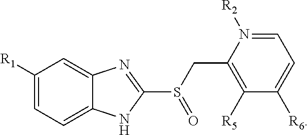

In certain embodiments, the compound of formula (II) is:

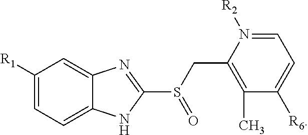

In particular embodiments, the compound of formula (II) is:

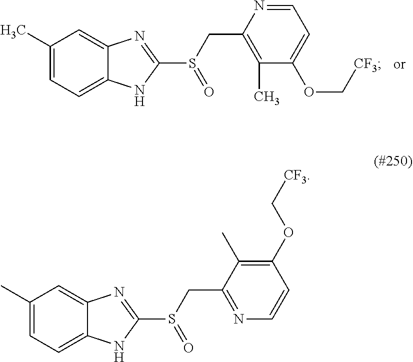 [SMILES: Cc1ccc2[nH]c(S(=O)Cc3nccc(OCC(F)(F)F)c3C)nc2c1 ; Cc1ccc2[nH]c(S(=O)Cc3nccc(OCC(F)(F)F)c3C)nc2c1]

In certain embodiments, the compound of formula (II) is:

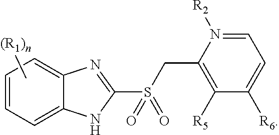

In certain embodiments, the compound of formula (II) is:

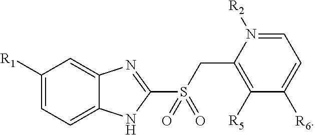

In certain embodiments, the compound of formula (II) is:

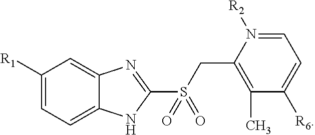

In particular embodiments, the compound of formula (II) is selected from the group consisting of:

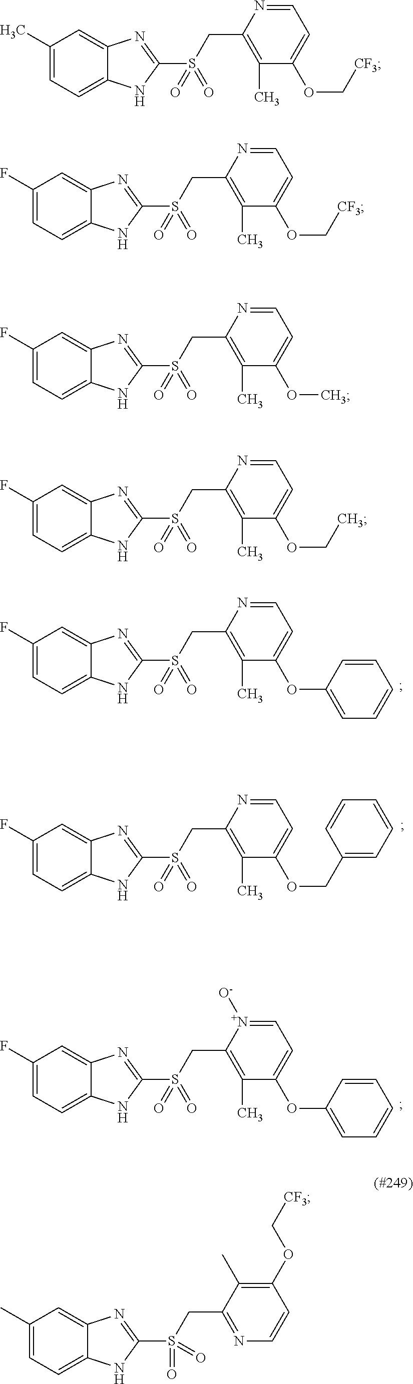

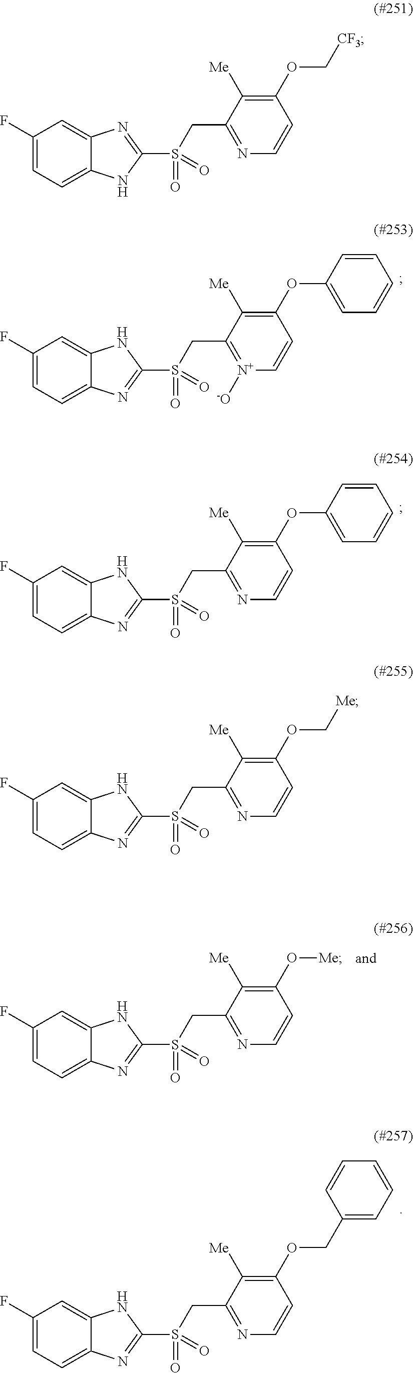

In some embodiments, the presently disclosed subject matter provides an aryl hydrocarbon receptor (AHR) agonist comprising a compound of formula (III):

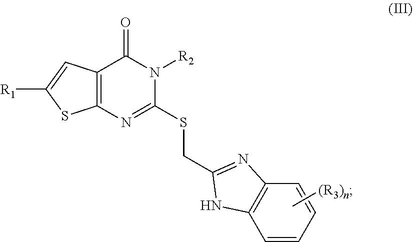

wherein:
n is an integer selected from the group consisting of 0, 1, 2, 3, and 4;
R₁ is substituted or unsubstituted straightchain or branched C₁-C₆ alkyl;
R₂ is selected from the group consisting of substituted aryl, unsubstituted aryl, substituted heteroaryl, and unsubstituted heteroaryal;
each R₃ is independently selected from the group consisting of H, substituted or unsubstituted straightchain or branched C₁-C₆ alkyl, hydroxyl, C₁-C₆ alkoxyl, halogen, —CF₃, cyano, amino, carboxyl, nitro, and mercapto; and
pharmaceutically acceptable salts thereof.

In certain embodiments, the compound of formula (III) is:

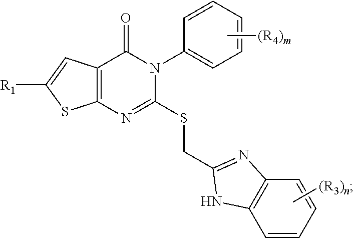

wherein:
   m is an integer selected from the group consisting of 0, 1, 2, 3, 4, and 5; and
   R₄ is selected from the group consisting of H, substituted or unsubstituted straightchain or branched C₁-C₆ alkyl, hydroxyl, C₁-C₆ alkoxyl, halogen, —CF₃, cyano, amino, carboxyl, nitro, and mercapto.

In particular embodiments, the compound of formula (III) is:

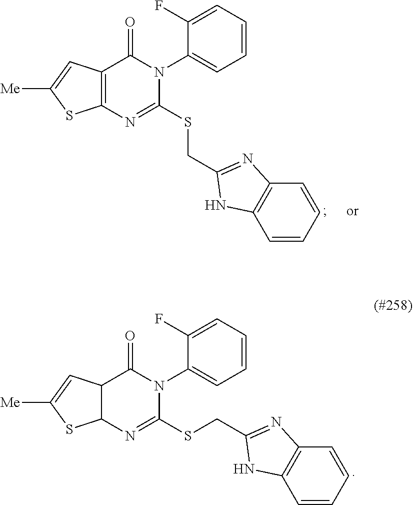 [SMILES: Cc1cc2c(=O)n(-c3ccccc3F)c(SCc3nc4ccccc4[nH]3)nc2s1 ; CC1=CC2C(=O)N(c3ccccc3F)C(SCc3nc4ccccc4[nH]3)=NC2S1]

### B. Methods of Treatment

In some embodiments, the presently disclosed subject matter provides a method for treating or preventing or reducing the risk of an inflammatory disorder associated with a reduced expression of an aryl hydrocarbon receptor (AHR) in a subject in need of treatment thereof, the method comprising administering to the subject one or more AHR agonists comprising a compound of formula (I-III), compound #169, which has the following structure:

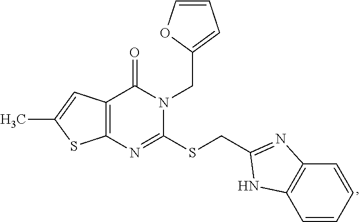 [SMILES: Cc1cc2c(=O)n(Cc3ccco3)c(SCc3nc4ccccc4[nH]3)nc2s1]

or pharmaceutically acceptable salts thereof, to activate the AHR, thereby treating or preventing or reducing the risk of the inflammatory disorder.

Inflammatory disorders include a large number of disorders or conditions that are involved in a variety of diseases, including those involving the immune system, including those demonstrated in allergic reactions and myopathies, or non-immune diseases with causal origins in inflammatory processes including, but not limited to cancer, atherosclerosis, and ischemic heart disease. Non-limiting examples of disorders associated with inflammation include, but are not limited to, acne vulgaris, asthma, autoimmune diseases, autoinflammatory diseases, celiac disease, chronic prostatitis, diverticulitis, glomerulonephritis, hidradenitis suppurativa, hypersensitivities, inflammatory bowel diseases, interstitial cystitis, otitis, pelvic inflammatory disease, reperfusion injury, rheumatic fever, rheumatoid arthritis, sarcoidosis, transplant rejection, and vasculitis.

More particularly, the presently disclosed subject matter may be used to treat any disease or disorder involving AHR activation, including, but not limited to, inflammatory disorders, such as necrotizing enterocolitis, inflammatory bowel disease, autoimmune diseases, Crohn's disease, celiac disease, ulcerative colitis, cardiovascular disease, ocular Behcet's disease, breast cancer, and others.

In particular embodiments, the inflammatory disorder is necrotizing enterocolitis.

As used herein, an agonist is an agent that binds to a receptor, e.g., AHR, and activates the receptor to produce a biological response.

The “subject” treated by the presently disclosed methods in their many embodiments is desirably a human subject, although it is to be understood that the methods described herein are effective with respect to all vertebrate species, which are intended to be included in the term “subject.” Accordingly, a “subject” can include a human subject for medical purposes, such as for the treatment of an existing condition or disease or the prophylactic treatment for preventing the onset of a condition or disease, or an animal subject for medical, veterinary purposes, or developmental purposes. Suitable animal subjects include mammals including, but not limited to, primates, e.g., humans, monkeys, apes, and the like; bovines, e.g., cattle, oxen, and the like; ovines, e.g., sheep and the like; caprines, e.g., goats and the like; porcines, e.g., pigs, hogs, and the like; equines, e.g., horses, donkeys, zebras, and the like; felines, including wild and domestic cats; canines, including dogs; lagomorphs, including rabbits, hares, and the like; and rodents, including mice, rats, and the like. An animal may be a transgenic animal. In some embodiments, the subject is a human including, but not limited to, fetal, neonatal, infant, juvenile, and adult subjects. Further, a “subject” can include a patient afflicted with or suspected of being afflicted with a condition or disease. Thus, the terms “subject” and “patient” are used interchangeably herein. The term “subject” also refers to an organism, tissue, cell, or collection of cells from a subject.

In some embodiments, the subject is a human subject. In particular embodiments, the subject is an infant. As used herein, the term “infant” can refer to a child from about one month after birth to one year after birth and can include a child up to about two years after birth.

As used herein, the term “newborn” or “neonate” refers to an infant in the first 28 days after birth, and can be an infant only a few hours after birth, a few days after birth, or up to a month after birth. The term applies generally to premature, full term (e.g., 38 weeks and beyond), and post mature infants.

In yet more particular embodiments, the human subject is a premature infant. As used herein; the term “premature birth” or “preterm birth” refers to the birth of a baby at fewer than 37 weeks gestational age and can include babies born at 37 weeks, 36 weeks, 35 weeks, 34 weeks, 33 weeks, 32 weeks, 31 weeks, 30 weeks, 29 weeks, 28 weeks, 27 weeks, 26 weeks, 25 weeks, 24 weeks, 23 weeks, 22 weeks, 21 weeks, and 20 weeks gestational age.

As used herein, “treatment” includes, without limitation, (1) decreasing the level of one or more index of inflammation (e.g., inflammatory cytokines, such as TNF-α, IL-6, IL-12p40, IL-113); (2) decreasing a clinical marker of inflammation, such as S100a8, Lcn2, iFabp, leukocyte count, fever, hypotension; and/or (3) reducing the risk of an adverse outcome, such as death, organ failure, hypoxia, or the need for surgery. “Treatment” does not necessarily mean that the condition being treated will be cured. A “therapeutically effective amount” of an AMR agonist achieves treatment.

“Reducing the risk” or “reducing the severity of does not necessarily mean that the subject being treated will not develop NEC. A “prophylactically effective amount” for preventing NEC reduces the risk of NEC by at least about ⅕ or by at least about ⅓. Any infant may be eligible for such prophylactic treatment. Infants at higher risk for NEC as a result of premature birth or low birth rate may particularly benefit, as well as a term infant otherwise at risk for NEC

Further, “methods of preventing” are defined as methods which reduce the risk of developing the disease, and do not necessarily result in 100% prevention of the disease. As such, these methods, applied prophylactically to an infant, may not only reduce the risk, but may also reduce the severity of the disease if it does occur. By definition, such preventative methods may be administered to an infant having no signs of preexisting NEC as well as to an infant which is exhibiting one or more early clinical sign consistent with NEC but in which a definitive diagnosis of NEC has not been established.

In some embodiments, the administration is enteral administration. As used herein, the term “enteral administration” includes feeding or drug administration through the gastrointestinal (GI) tract. Enteral administration can include oral administration or gastric administration, for example, via a feeding tube through the nasal passage, i.e., a nasogastric (NG) tube, or a feeding tube leading directly (the stomach, i.e., a percutaneous endoscopic gastrostomy (PEG) tube.

In general, the “effective amount” of an active agent or drug delivery device refers to the amount necessary to elicit the desired biological response. As will be appreciated by those of ordinary skill in this art, the effective amount of an agent or device may vary depending on such factors as the desired biological endpoint, the agent to be delivered, the makeup of the pharmaceutical composition, the target tissue, and the like.

The term “combination” is used in its broadest sense and means that a subject is administered at least two agents, more particularly a compound of formula (I-III), and/or compound #169, and one or more therapeutic agents. More particularly, the term “in combination” refers to the concomitant administration of two (or more) active agents for the treatment of a, e.g., single disease state. As used herein, the active agents may be combined and administered in a single dosage form, may be administered as separate dosage forms at the same time, or may be administered as separate dosage forms that are administered alternately or sequentially on the same or separate days. In one embodiment of the presently disclosed subject matter, the active agents are combined and administered in a single dosage form. In another embodiment, the active agents are administered in separate dosage forms (e.g., wherein it is desirable to vary the amount of one but not the other). The single dosage form may include additional active agents for the treatment of the disease state.

In some embodiments, the one or more therapeutic agents comprise one or more AHR agonists. In some embodiments, the one or more AHR agonists is selected from the group consisting of abacavir, abacavir sulfate, amlexanox, anagrelide hydrochloride, benzocaine (ethyl p-aminobenzoate), bromindione, catharanthine, dexlansoprazole, eseroline, febuxostat, helenien (xantofyl palmitate), hydralazine hydrochloride, indoprofen, ipratropium bromide, lansoprazole, menadione sodium bisulfate, nitazoxanide, omeprazole, phenazopyridine, phenazopyridine hydrochloride, primaquine, rabeprazole sodium, tenatoprazole, tranilast (sb 252218), and ziprasidone hydrochloride, indole-3-carbinol (I3C), A18, or derivatives and combinations thereof, or pharmaceutically acceptable salts thereof.

Further, the compounds of formula (I-III) and/or compound #169 described herein can be administered alone or in combination with adjuvants that enhance stability of the compounds of formula (I-III) and/or compound #169, facilitate administration of pharmaceutical compositions containing them in certain embodiments, provide increased dissolution or dispersion, increase inhibitory activity, provide adjunct therapy, and the like, including other active ingredients. Advantageously, such combination therapies utilize lower dosages of the conventional therapeutics, thus avoiding possible toxicity and adverse side effects incurred when those agents are used as monotherapies.

The timing of administration of a compound of formula (I-III) and/or compound #169 and at least one additional therapeutic agent can be varied so long as the beneficial effects of the combination of these agents are achieved. Accordingly, the phrase “in combination with” refers to the administration of a compound of formula (I-III) and/or compound #169 and at least one additional therapeutic agent either simultaneously, sequentially, or a combination thereof. Therefore, a subject administered a combination of a compound of formula (I-III) and/or compound #169 and at least one additional therapeutic agent can receive compound of formula (I-III) and/or compound #169 and at least one additional therapeutic agent at the same time (i.e., simultaneously) or at different times (i.e., sequentially, in either order, on the same day or on different days), so long as the effect of the combination of both agents is achieved in the subject.

When administered sequentially, the agents can be administered within 1, 5, 10, 30, 60, 120, 180, 240 minutes or longer of one another. In other embodiments, agents administered sequentially, can be administered within 1, 5, 10, 15, 20 or more days of one another. Where the compound of formula (I-III) and/or compound #169 and at least one additional therapeutic agent are administered simultaneously, they can be administered to the subject as separate pharmaceutical compositions, each comprising either a compound of formula (I-III) and/or compound #169 or at least one additional therapeutic agent, or they can be administered to a subject as a single pharmaceutical composition comprising both agents.

When administered in combination, the effective concentration of each of the agents to elicit a particular biological response may be less than the effective concentration of each agent when administered alone, thereby allowing a reduction in the dose of one or more of the agents relative to the dose that would be needed if the agent was administered as a single agent. The effects of multiple agents may, but need not be, additive or synergistic. The agents may be administered multiple times.

In some embodiments, when administered in combination, the two or more agents can have a synergistic effect. As used herein, the terms “synergy,” “synergistic,” “synergistically” and derivations thereof, such as in a “synergistic effect” or a “synergistic combination” or a “synergistic composition” refer to circumstances under which the biological activity of a combination of a compound of formula (I-III) and/or compound #169 and at least one additional therapeutic agent is greater than the sum of the biological activities of the respective agents when administered individually.

Synergy can be expressed in terms of a “Synergy Index (SI),” which generally can be determined by the method described by F. C. Kull et al., Applied Microbiology 9, 538 (1961), from the ratio determined by:

Qₐ/Q_{A}+Q_{b}/Q_{B}=Synergy Index(SI)

wherein:
Q_{A} is the concentration of a component A, acting alone, which produced an end point in relation to component A;
Qₐ is the concentration of component A, in a mixture, which produced an end point;
Q_{B} is the concentration of a component B, acting alone, which produced an end point in relation to component B; and
Q_{b} is the concentration of component B, in a mixture, which produced an end point.

Generally, when the sum of Qₐ/Q_{A} and Q_{b}/Q_{B} is greater than one, antagonism is indicated. When the sum is equal to one, additivity is indicated. When the sum is less than one, synergism is demonstrated. The lower the SI, the greater the synergy shown by that particular mixture. Thus, a “synergistic combination” has an activity higher that what can be expected based on the observed activities of the individual components when used alone. Further, a “synergistically effective amount” of a component refers to the amount of the component necessary to elicit a synergistic effect in, for example, another therapeutic agent present in the composition.

### C. Infant Nutritional Formula Comprising Aryl Hydrocarbon Receptor (AHR) Agonists for the Prevention and Treatment of Inflammatory Disorders

In some embodiments, the presently disclosed subject matter provides an infant nutritional formula comprising a therapeutically effective amount of one or more aryl hydrocarbon receptor (AHR) agonists comprising a compound of formula (I-III), and/or compound #169, or pharmaceutically acceptable salts thereof.

In certain embodiments, the formula is nutritionally complete, that is it is suitable as a sole source of nutrition for a premature or full term infant. In such embodiments, the formula comprises one or more ingredients selected from the group consisting of protein, fat, one or more fatty acids, such as linoleic acid, and/or oleic acid, and/or other fatty acids, vitamin A, vitamin C, vitamin D, vitamin E, vitamin K, thiamin (B1), riboflavin (B2), B6, B12, niacin, folic acid, pantothenic acid, calcium, magnesium, iron, zinc, manganese, copper, phosphorous, iodine, sodium chloride, potassium chloride, one or more carbohydrates, such as oligosaccharides, such as milk oligosaccharides, and other complex or simple sugars, including lactose, sucrose, glucose, dextrins, natural and modified starches, cholesterol, phospholipid, casein, whey, soy protein, nucleotides, emulsifiers, stabilizers, and diluents.

In some embodiments, the formula is in the form of a liquid, a powder, a capsule, a tablet, or an orally disintegrating tablet. In particular embodiments, the liquid is in the form of a solution, an emulsion, or a suspension.

In certain embodiments, where the formulation is a liquid, the formulation comprises a pharmaceutically suitable liquid such as, but not limited to, water, saline, or an emulsion formed between an aqueous solution and an oil or other liquid that is not substantially miscible with water. In a specific non-limiting embodiment a liquid formulation may comprise a hydrophobic compound as well as an emulsifier.

In certain non-limiting embodiments, one or more AHR agonists can be added to a commercial infant nutritional formula prior to administration, for example, but not limited to, Similac®, Enfamil® or Gerber® formulas. In specific non-limiting embodiments such formula may be Similac® Premature Infant Formula, Enfamil®Premature LIPIL, or Gerber® Good Start, or similar commercially available infant formulas formulated for premature infants.

In other embodiments, the one or more AHR agonists can be included in an infant nutritional formula that has not yet been commercially available, where the infant nutritional formula further comprises one or more nutrients, such as proteins, lipids, carbohydrates, electrolytes, and/or vitamins as provided hereinabove. The infant nutritional formula may be, without limitation, a liquid or a powder for reconstitution with liquid.

In one specific non-limiting embodiment the one or more AHR agonists may be added to other components of the formulation shortly prior to use, for example within 24 hours or within 6 hours or within 2 hours or within 1 hour of use. In certain embodiments, the formula is adapted for enteral administration to an infant. In more certain embodiments, the enteral administration is oral administration or gastric administration.

### D. Maternal Administration of an Aryl Hydrocarbon Receptor (AHR) Agonist

In some embodiments, the presently disclosed subject matter provides a method for preventing, reducing the risk of, or reducing the severity of an inflammatory disorder associated with a reduced expression of an aryl hydrocarbon receptor (AHR) in a subject in need of treatment thereof, the method comprising administering to a mother while pregnant with the subject one or more AHR agonists comprising a compound of formula (I-III), and/or compound #169 or pharmaceutically acceptable salts thereof, to activate the AHR, thereby treating or preventing, reducing the risk of, or reducing the severity of the inflammatory disorder.

In some embodiments, the mother is at risk for delivering the subject prematurely.

### E. Pharmaceutical Compositions and Administration

In another aspect, the present disclosure provides a pharmaceutical composition including one compound of formula (I-III) and/or compound #169 alone or in combination with one or more additional therapeutic agents in admixture with a pharmaceutically acceptable excipient. One of skill in the art will recognize that the pharmaceutical compositions include the pharmaceutically acceptable salts of the compounds described above. Pharmaceutically acceptable salts are generally well known to those of ordinary skill in the art, and include salts of active compounds which are prepared with relatively nontoxic acids or bases, depending on the particular substituent moieties found on the compounds described herein. When compounds of the present disclosure contain relatively acidic functionalities, base addition salts can be obtained by contacting the neutral form of such compounds with a sufficient amount of the desired base, either neat or in a suitable inert solvent or by ion exchange, whereby one basic counterion (base) in an ionic complex is substituted for another. Examples of pharmaceutically acceptable base addition salts include sodium, potassium, calcium, ammonium, organic amino, or magnesium salt, or a similar salt.

When compounds of the present disclosure contain relatively basic functionalities, acid addition salts can be obtained by contacting the neutral form of such compounds with a sufficient amount of the desired acid, either neat or in a suitable inert solvent or by ion exchange, whereby one acidic counterion (acid) in an ionic complex is substituted for another. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-toluenesulfonic, citric, tartaric, methanesulfonic, and the like. Also included are salts of amino acids such as arginate and the like, and salts of organic acids like glucuronic or galactunoric acids and the like (see, for example, Berge et al, “Pharmaceutical Salts”, Journal of Pharmaceutical Science, 1977, 66, 1-19). Certain specific compounds of the present disclosure contain both basic and acidic functionalities that allow the compounds to be converted into either base or acid addition salts.

Accordingly, pharmaceutically acceptable salts suitable for use with the presently disclosed subject matter include, by way of example but not limitation, acetate, benzenesulfonate, benzoate, bicarbonate, bitartrate, bromide, calcium edetate, camsylate, carbonate, citrate, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isethionate, lactate, lactobionate, malate, maleate, mandelate, mesylate, mucate, napsylate, nitrate, pamoate (embonate), pantothenate, phosphate/diphosphate, polygalacturonate, salicylate, stearate, subacetate, succinate, sulfate, tannate, tartrate, or teoclate. Other pharmaceutically acceptable salts may be found in, for example, Remington: The Science and Practice of Pharmacy (20^{th} ed.) Lippincott, Williams & Wilkins (2000).

In therapeutic and/or diagnostic applications, the compounds of the disclosure can be formulated for a variety of modes of administration, including systemic and topical or localized administration. Techniques and formulations generally may be found in Remington: The Science and Practice of Pharmacy (20^{th} ed.) Lippincott, Williams & Wilkins (2000).

Depending on the specific conditions being treated, such agents may be formulated into liquid or solid dosage forms and administered systemically or locally. The agents may be delivered, for example, in a timed- or sustained-slow release form as is known to those skilled in the art. Techniques for formulation and administration may be found in Remington: The Science and Practice of Pharmacy (20^{th} ed.) Lippincott, Williams & Wilkins (2000). Suitable routes may include oral, buccal, by inhalation spray, sublingual, rectal, transdermal, vaginal, transmucosal, nasal or intestinal administration; parenteral delivery, including intramuscular, subcutaneous, intramedullary injections, as well as intrathecal, direct intraventricular, intravenous, intra-articular, intra-sternal, intra-synovial, intra-hepatic, intralesional, intracranial, intraperitoneal, intranasal, or intraocular injections or other modes of delivery.

For injection, the agents of the disclosure may be formulated and diluted in aqueous solutions, such as in physiologically compatible buffers such as Hank's solution, Ringer's solution, or physiological saline buffer. For such transmucosal administration, penetrants appropriate to the barrier to be permeated are used in the formulation. Such penetrants are generally known in the art.

Use of pharmaceutically acceptable inert carriers to formulate the compounds herein disclosed for the practice of the disclosure into dosages suitable for systemic administration is within the scope of the disclosure. With proper choice of carrier and suitable manufacturing practice, the compositions of the present disclosure, in particular, those formulated as solutions, may be administered parenterally, such as by intravenous injection. The compounds can be formulated readily using pharmaceutically acceptable carriers well known in the art into dosages suitable for oral administration. Such carriers enable the compounds of the disclosure to be formulated as tablets, pills, capsules, liquids, gels, syrups, slurries, suspensions and the like, for oral ingestion by a subject (e.g., patient) to be treated.

For nasal or inhalation delivery, the agents of the disclosure also may be formulated by methods known to those of skill in the art, and may include, for example, but not limited to, examples of solubilizing, diluting, or dispersing substances, such as saline; preservatives, such as benzyl alcohol; absorption promoters; and fluorocarbons.

Pharmaceutical compositions suitable for use in the present disclosure include compositions wherein the active ingredients are contained in an effective amount to achieve its intended purpose. Determination of the effective amounts is well within the capability of those skilled in the art, especially in light of the detailed disclosure provided herein. Generally, the compounds according to the disclosure are effective over a wide dosage range. For example, in the treatment of adult humans, dosages from 0.01 to 1000 mg, from 0.5 to 100 mg, from 1 to 50 mg per day, and from 5 to 40 mg per day are examples of dosages that may be used. A non-limiting dosage is 10 to 30 mg per day. The exact dosage will depend upon the route of administration, the form in which the compound is administered, the subject to be treated, the body weight of the subject to be treated, the bioavailability of the compound(s), the adsorption, distribution, metabolism, and excretion (ADME) toxicity of the compound(s), and the preference and experience of the attending physician.

In addition to the active ingredients, these pharmaceutical compositions may contain suitable pharmaceutically acceptable carriers comprising excipients and auxiliaries which facilitate processing of the active compounds into preparations which can be used pharmaceutically. The preparations formulated for oral administration may be in the form of tablets, dragees, capsules, or solutions.

Pharmaceutical preparations for oral use can be obtained by combining the active compounds with solid excipients, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations, for example, maize starch, wheat starch, rice starch, potato starch, gelatin, gum tragacanth, methyl cellulose, hydroxypropylmethyl-cellulose, sodium carboxymethyl-cellulose (CMC), and/or polyvinylpyrrolidone (PVP: povidone). If desired, disintegrating agents may be added, such as the cross-linked polyvinylpyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinylpyrrolidone, carbopol gel, polyethylene glycol (PEG), and/or titanium dioxide, lacquer solutions, and suitable organic solvents or solvent mixtures. Dye-stuffs or pigments may be added to the tablets or dragee coatings for identification or to characterize different combinations of active compound doses.

Pharmaceutical preparations that can be used orally include push-fit capsules made of gelatin, as well as soft, sealed capsules made of gelatin, and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols (PEGs). In addition, stabilizers may be added.

### F. Definitions

Although specific terms are employed herein, they are used in a generic and descriptive sense only and not for purposes of limitation. Unless otherwise defined, all technical and scientific terms used herein have the same meaning as commonly understood by one of ordinary skill in the art to which this presently described subject matter belongs.

While the following terms in relation to compounds of formula (I-III) are believed to be well understood by one of ordinary skill in the art, the following definitions are set forth to facilitate explanation of the presently disclosed subject matter. These definitions are intended to supplement and illustrate, not preclude, the definitions that would be apparent to one of ordinary skill in the art upon review of the present disclosure.

The terms substituted, whether preceded by the term “optionally” or not, and substituent, as used herein, refer to the ability, as appreciated by one skilled in this art, to change one functional group for another functional group on a molecule, provided that the valency of all atoms is maintained. When more than one position in any given structure may be substituted with more than one substituent selected from a specified group, the substituent may be either the same or different at every position. The substituents also may be further substituted (e.g., an aryl group substituent may have another substituent off it, such as another aryl group, which is further substituted at one or more positions).

Where substituent groups or linking groups are specified by their conventional chemical formulae, written from left to right, they equally encompass the chemically identical substituents that would result from writing the structure from right to left, e.g., —CH₂O— is equivalent to —OCH₂—; —C(═O)O— is equivalent to —OC(═O)—; —OC(═O)NR— is equivalent to —NRC(═O)O—, and the like.

When the term “independently selected” is used, the substituents being referred to (e.g., R groups, such as groups R₁, R₂, and the like, or variables, such as “m” and “n”), can be identical or different. For example, both R₁ and R₂ can be substituted alkyls, or R₁ can be hydrogen and R₂ can be a substituted alkyl, and the like.

The terms “a,” “an,” or “a(n),” when used in reference to a group of substituents herein, mean at least one. For example, where a compound is substituted with “an” alkyl or aryl, the compound is optionally substituted with at least one alkyl and/or at least one aryl. Moreover, where a moiety is substituted with an R substituent, the group may be referred to as “R-substituted.” Where a moiety is R-substituted, the moiety is substituted with at least one R substituent and each R substituent is optionally different.

A named “R” or group will generally have the structure that is recognized in the art as corresponding to a group having that name, unless specified otherwise herein. For the purposes of illustration, certain representative “R” groups as set forth above are defined below.

Descriptions of compounds of the present disclosure are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

Unless otherwise explicitly defined, a “substituent group,” as used herein, includes a functional group selected from one or more of the following moieties, which are defined herein:

The term hydrocarbon, as used herein, refers to any chemical group comprising hydrogen and carbon. The hydrocarbon may be substituted or unsubstituted. As would be known to one skilled in this art, all valencies must be satisfied in making any substitutions. The hydrocarbon may be unsaturated, saturated, branched, unbranched, cyclic, polycyclic, or heterocyclic. Illustrative hydrocarbons are further defined herein below and include, for example, methyl, ethyl, n-propyl, isopropyl, cyclopropyl, allyl, vinyl, n-butyl, tert-butyl, ethynyl, cyclohexyl, and the like.

The term “alkyl,” by itself or as part of another substituent, means, unless otherwise stated, a straight (i.e., unbranched) or branched chain, acyclic or cyclic hydrocarbon group, or combination thereof, which may be fully saturated, mono- or polyunsaturated and can include di- and multivalent groups, having the number of carbon atoms designated (i.e., C₁₋₁₀ means one to ten carbons, including 1, 2, 3, 4, 5, 6, 7, 8, 9, and 10 carbons). In particular embodiments, the term “alkyl” refers to C₁₋₂₀ inclusive, including 1, 2, 3, 4, 5, 6, 7, 8, 9, 10, 11, 12, 13, 14, 15, 16, 17, 18, 19, and 20 carbons, linear (i.e., “straight-chain”), branched, or cyclic, saturated or at least partially and in some cases fully unsaturated (i.e., alkenyl and alkynyl) hydrocarbon radicals derived from a hydrocarbon moiety containing between one and twenty carbon atoms by removal of a single hydrogen atom. In particular embodiments, the alkyl group comprises a C₁-C₆ alkyl group, including up to 1, 2, 3, 4, 5, and 6 carbon atoms.

Representative saturated hydrocarbon groups include, but are not limited to, methyl, ethyl, n-propyl, isopropyl, n-butyl, isobutyl, sec-butyl, tert-butyl, n-pentyl, sec-pentyl, isopentyl, neopentyl, n-hexyl, sec-hexyl, n-heptyl, n-octyl, n-decyl, n-undecyl, dodecyl, cyclohexyl, (cyclohexyl)methyl, cyclopropylmethyl, and homologs and isomers thereof.

“Branched” refers to an alkyl group in which a lower alkyl group, such as methyl, ethyl or propyl, is attached to a linear alkyl chain. “Lower alkyl” refers to an alkyl group having 1 to about 8 carbon atoms (i.e., a C₁₋₈ alkyl), e.g., 1, 2, 3, 4, 5, 6, 7, or 8 carbon atoms. “Higher alkyl” refers to an alkyl group having about 10 to about 20 carbon atoms, e.g., 10, 11, 12, 13, 14, 15, 16, 17, 18, 19, or 20 carbon atoms. In certain embodiments, “alkyl” refers, in particular, to C₁₋₈ straight-chain alkyls. In other embodiments, “alkyl” refers, in particular, to C₁₋₈ branched-chain alkyls.

Alkyl groups can optionally be substituted (a “substituted alkyl”) with one or more alkyl group substituents, which can be the same or different. The term “alkyl group substituent” includes but is not limited to alkyl, substituted alkyl, halo, arylamino, acyl, hydroxyl, aryloxyl, alkoxyl, alkylthio, arylthio, aralkyloxyl, aralkylthio, carboxyl, alkoxycarbonyl, oxo, and cycloalkyl. There can be optionally inserted along the alkyl chain one or more oxygen, sulfur or substituted or unsubstituted nitrogen atoms, wherein the nitrogen substituent is hydrogen, lower alkyl (also referred to herein as “alkylaminoalkyl”), or aryl.

Thus, as used herein, the term “substituted alkyl” includes alkyl groups, as defined herein, in which one or more atoms or functional groups of the alkyl group are replaced with another atom or functional group, including for example, alkyl, substituted alkyl, halogen, aryl, substituted aryl, alkoxyl, hydroxyl, nitro, amino, alkylamino, dialkylamino, sulfate, cyano, and mercapto.

The term “heteroalkyl,” by itself or in combination with another term, means, unless otherwise stated, a stable straight or branched chain having from 1 to 20 carbon atoms or heteroatoms or a cyclic hydrocarbon group having from 3 to 10 carbon atoms or heteroatoms, or combinations thereof, consisting of at least one carbon atom and at least one heteroatom selected from the group consisting of O, N, P, Si and S, and wherein the nitrogen, phosphorus, and sulfur atoms may optionally be oxidized and the nitrogen heteroatom may optionally be quaternized. The heteroatom(s) O, N, P and S and Si may be placed at any interior position of the heteroalkyl group or at the position at which alkyl group is attached to the remainder of the molecule. Examples include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂—S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)— CH₃, O—CH₃, —O—CH₂—CH₃, and —CN. Up to two or three heteroatoms may be consecutive, such as, for example, —CH₂—NH—OCH₃ and —CH₂—O—Si(CH₃)₃.

As described above, heteroalkyl groups, as used herein, include those groups that are attached to the remainder of the molecule through a heteroatom, such as —C(O)NR′, —NR′R″, —OR′, —SR, —S(O)R, and/or —S(O₂)R′. Where “heteroalkyl” is recited, followed by recitations of specific heteroalkyl groups, such as —NR′R or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

“Cyclic” and “cycloalkyl” refer to a non-aromatic mono- or multicyclic ring system of about 3 to about 10 carbon atoms, e.g., 3, 4, 5, 6, 7, 8, 9, or 10 carbon atoms. The cycloalkyl group can be optionally partially unsaturated. The cycloalkyl group also can be optionally substituted with an alkyl group substituent as defined herein, oxo, and/or alkylene. There can be optionally inserted along the cyclic alkyl chain one or more oxygen, sulfur or substituted or unsubstituted nitrogen atoms, wherein the nitrogen substituent is hydrogen, unsubstituted alkyl, substituted alkyl, aryl, or substituted aryl, thus providing a heterocyclic group. Representative monocyclic cycloalkyl rings include cyclopentyl, cyclohexyl, and cycloheptyl. Multicyclic cycloalkyl rings include adamantyl, octahydronaphthyl, decalin, camphor, camphane, and noradamantyl, and fused ring systems, such as dihydro- and tetrahydronaphthalene, and the like.

The term “cycloalkylalkyl,” as used herein, refers to a cycloalkyl group as defined hereinabove, which is attached to the parent molecular moiety through an alkylene moiety, also as defined above, e.g., a C₁₋₂₀ alkylene moiety. Examples of cycloalkylalkyl groups include cyclopropylmethyl and cyclopentylethyl.

The terms “cycloheteroalkyl” or “heterocycloalkyl” refer to a non-aromatic ring system, unsaturated or partially unsaturated ring system, such as a 3- to 10-member substituted or unsubstituted cycloalkyl ring system, including one or more heteroatoms, which can be the same or different, and are selected from the group consisting of nitrogen (N), oxygen (O), sulfur (S), phosphorus (P), and silicon (Si), and optionally can include one or more double bonds.

The cycloheteroalkyl ring can be optionally fused to or otherwise attached to other cycloheteroalkyl rings and/or non-aromatic hydrocarbon rings. Heterocyclic rings include those having from one to three heteroatoms independently selected from oxygen, sulfur, and nitrogen, in which the nitrogen and sulfur heteroatoms may optionally be oxidized and the nitrogen heteroatom may optionally be quaternized. In certain embodiments, the term heterocylic refers to a non-aromatic 5-, 6-, or 7-membered ring or a polycyclic group wherein at least one ring atom is a heteroatom selected from O, S, and N (wherein the nitrogen and sulfur heteroatoms may be optionally oxidized), including, but not limited to, a bi- or tri-cyclic group, comprising fused six-membered rings having between one and three heteroatoms independently selected from the oxygen, sulfur, and nitrogen, wherein (i) each 5-membered ring has 0 to 2 double bonds, each 6-membered ring has 0 to 2 double bonds, and each 7-membered ring has 0 to 3 double bonds, (ii) the nitrogen and sulfur heteroatoms may be optionally oxidized, (iii) the nitrogen heteroatom may optionally be quaternized, and (iv) any of the above heterocyclic rings may be fused to an aryl or heteroaryl ring. Representative cycloheteroalkyl ring systems include, but are not limited to pyrrolidinyl, pyrrolinyl, imidazolidinyl, imidazolinyl, pyrazolidinyl, pyrazolinyl, piperidinyl, piperazinyl, indolinyl, quinuclidinyl, morpholinyl, thiomorpholinyl, thiadiazinanyl, tetrahydrofuranyl, and the like.

The terms “cycloalkyl” and “heterocycloalkyl”, by themselves or in combination with other terms, represent, unless otherwise stated, cyclic versions of “alkyl” and “heteroalkyl”, respectively. Additionally, for heterocycloalkyl, a heteroatom can occupy the position at which the heterocycle is attached to the remainder of the molecule. Examples of cycloalkyl include, but are not limited to, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. The terms “cycloalkylene” and “heterocycloalkylene” refer to the divalent derivatives of cycloalkyl and heterocycloalkyl, respectively.

An unsaturated hydrocarbon has one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. Alkyl groups which are limited to hydrocarbon groups are termed “homoalkyl.”

More particularly, the term “alkenyl” as used herein refers to a monovalent group derived from a C₂₋₂₀ inclusive straight or branched hydrocarbon moiety having at least one carbon-carbon double bond by the removal of a single hydrogen molecule. Alkenyl groups include, for example, ethenyl (i.e., vinyl), propenyl, butenyl, 1-methyl-2-buten-1-yl, pentenyl, hexenyl, octenyl, allenyl, and butadienyl.

The term “cycloalkenyl” as used herein refers to a cyclic hydrocarbon containing at least one carbon-carbon double bond. Examples of cycloalkenyl groups include cyclopropenyl, cyclobutenyl, cyclopentenyl, cyclopentadiene, cyclohexenyl, 1,3-cyclohexadiene, cycloheptenyl, cycloheptatrienyl, and cyclooctenyl.

The term “alkynyl” as used herein refers to a monovalent group derived from a straight or branched C₂₋₂₀ hydrocarbon of a designed number of carbon atoms containing at least one carbon-carbon triple bond. Examples of “alkynyl” include ethynyl, 2-propynyl (propargyl), 1-propynyl, pentynyl, hexynyl, and heptynyl groups, and the like.

The term “alkylene” by itself or a part of another substituent refers to a straight or branched bivalent aliphatic hydrocarbon group derived from an alkyl group having from 1 to about 20 carbon atoms, e.g., 1, 2, 3, 4, 5, 6, 7, 8, 9, 10, 11, 12, 13, 14, 15, 16, 17, 18, 19, or 20 carbon atoms. The alkylene group can be straight, branched or cyclic. The alkylene group also can be optionally unsaturated and/or substituted with one or more “alkyl group substituents.” There can be optionally inserted along the alkylene group one or more oxygen, sulfur or substituted or unsubstituted nitrogen atoms (also referred to herein as “alkylaminoalkyl”), wherein the nitrogen substituent is alkyl as previously described. Exemplary alkylene groups include methylene (—CH₂—); ethylene (—CH₂—CH₂—); propylene (—(CH₂)₃—); cyclohexylene (—C₆H₁₀); —CH═CH—CH═CH—; —CH═CH—CH₂—; —CH₂CH₂CH₂CH₂—, —CH₂CH═CHCH₂—, —CH₂CsCCH₂—, —CH₂CH₂CH(CH₂CH₂CH₃)CH₂—, —(CH₂)_{q}-N(R)—(CH₂)ᵣ—, wherein each of q and r is independently an integer from 0 to about 20, e.g., 0, 1, 2, 3, 4, 5, 6, 7, 8, 9, 10, 11, 12, 13, 14, 15, 16, 17, 18, 19, or 20, and R is hydrogen or lower alkyl; methylenedioxyl (—O—CH₂—O—); and ethylenedioxyl (—O—(CH₂)₂—O—). An alkylene group can have about 2 to about 3 carbon atoms and can further have 6-20 carbons. Typically, an alkyl (or alkylene) group will have from 1 to 24 carbon atoms, with those groups having 10 or fewer carbon atoms being some embodiments of the present disclosure. A “lower alkyl” or “lower alkylene” is a shorter chain alkyl or alkylene group, generally having eight or fewer carbon atoms.

The term “heteroalkylene” by itself or as part of another substituent means a divalent group derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms also can occupy either or both of the chain termini (e.g., alkyleneoxo, alkylenedioxo, alkyleneamino, alkylenediamino, and the like). Still further, for alkylene and heteroalkylene linking groups, no orientation of the linking group is implied by the direction in which the formula of the linking group is written. For example, the formula —C(O)OR′— represents both —C(O)OR′— and —R′OC(O)—.

The term “aryl” means, unless otherwise stated, an aromatic hydrocarbon substituent that can be a single ring or multiple rings (such as from 1 to 3 rings), which are fused together or linked covalently. The term “heteroaryl” refers to aryl groups (or rings) that contain from one to four heteroatoms (in each separate ring in the case of multiple rings) selected from N, O, and S, wherein the nitrogen and sulfur atoms are optionally oxidized, and the nitrogen atom(s) are optionally quaternized. A heteroaryl group can be attached to the remainder of the molecule through a carbon or heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of above noted aryl and heteroaryl ring systems are selected from the group of acceptable substituents described below. The terms “arylene” and “heteroarylene” refer to the divalent forms of aryl and heteroaryl, respectively.

For brevity, the term “aryl” when used in combination with other terms (e.g., aryloxy, arylthioxy, arylalkyl) includes both aryl and heteroaryl rings as defined above. Thus, the terms “arylalkyl” and “heteroarylalkyl” are meant to include those groups in which an aryl or heteroaryl group is attached to an alkyl group (e.g., benzyl, phenethyl, pyridylmethyl, furylmethyl, and the like) including those alkyl groups in which a carbon atom (e.g., a methylene group) has been replaced by, for example, an oxygen atom (e.g., phenoxymethyl, 2-pyridyloxymethyl, 3-(1-naphthyloxy)propyl, and the like). However, the term “haloaryl,” as used herein is meant to cover only aryls substituted with one or more halogens.

Where a heteroalkyl, heterocycloalkyl, or heteroaryl includes a specific number of members (e.g. “3 to 7 membered”), the term “member” refers to a carbon or heteroatom.

Further, a structure represented generally by the formula:

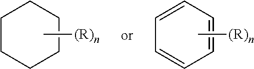

as used herein refers to a ring structure, for example, but not limited to a 3-carbon, a 4-carbon, a 5-carbon, a 6-carbon, a 7-carbon, and the like, aliphatic and/or aromatic cyclic compound, including a saturated ring structure, a partially saturated ring structure, and an unsaturated ring structure, comprising a substituent R group, wherein the R group can be present or absent, and when present, one or more R groups can each be substituted on one or more available carbon atoms of the ring structure. The presence or absence of the R group and number of R groups is determined by the value of the variable “n,” which is an integer generally having a value ranging from 0 to the number of carbon atoms on the ring available for substitution. Each R group, if more than one, is substituted on an available carbon of the ring structure rather than on another R group. For example, the structure above where n is 0 to 2 would comprise compound groups including, but not limited to:

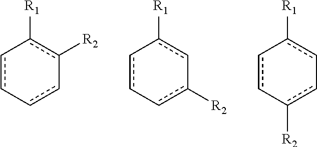

and the like.

A dashed line ( [symbol] ) representing a bond in a structure indicates that the bond can be either present or absent. For example, a dashed line representing a bond in a cyclic ring structure indicates that the ring structure is selected from the group consisting of a saturated ring structure, a partially saturated ring structure, and an unsaturated ring structure. A dashed line in an alkylene group indicates that the group is an alkylene group or an alkylene group comprising a double bond.

The symbol ( [symbol] ) denotes the point of attachment of a moiety to the remainder of the molecule.

When a named atom of an aromatic ring or a heterocyclic aromatic ring is defined as being “absent,” the named atom is replaced by a direct bond.

Each of above terms (e.g., “alkyl,” “heteroalkyl,” “cycloalkyl, and “heterocycloalkyl”, “aryl,” “heteroaryl,” “phosphonate,” and “sulfonate” as well as their divalent derivatives) are meant to include both substituted and unsubstituted forms of the indicated group. Optional substituents for each type of group are provided below.

Substituents for alkyl, heteroalkyl, cycloalkyl, heterocycloalkyl monovalent and divalent derivative groups (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to: —OR′, ═O, ═NR′, ═N—OR′, —NR′R″, —SR′, -halogen, —SiR′R″R′″, —OC(O)R′, —C(O)R′, —CO₂R′, —C(O)NR′R″, —OC(O)NR′R″, —NR″C(O)R′, —NR′—C(O)NR″R′″, —NR″C(O)OR′, —NR—C(NR′R″)═NR′″, —S(O)R′, —S(O)₂R′, —S(O)₂NR′R″, —NRSO₂R′, —CN, CF₃, fluorinated C₁₋₄ alkyl, and —NO₂ in a number ranging from zero to (2m′+1), where m′ is the total number of carbon atoms in such groups. R′, R″, R′″ and R″″ each may independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted alkyl, alkoxy or thioalkoxy groups, or arylalkyl groups. As used herein, an “alkoxy” group is an alkyl attached to the remainder of the molecule through a divalent oxygen. When a compound of the disclosure includes more than one R group, for example, each of the R groups is independently selected as are each R′, R′″, R″ and R″″ groups when more than one of these groups is present. When R′ and R″ are attached to the same nitrogen atom, they can be combined with the nitrogen atom to form a 4-, 5-, 6-, or 7-membered ring. For example, —NR′R″ is meant to include, but not be limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for alkyl groups above, exemplary substituents for aryl and heteroaryl groups (as well as their divalent derivatives) are varied and are selected from, for example: halogen, —OR′, —NR′R″, —SR′, —SiR′R″R′″, —OC(O)R′, —C(O)R′, —CO₂R′, —C(O)NR′R″, —OC(O)NR′R″, —NR″C(O)R′, —NR′—C(O)NR″R′″, —NR″C(O)OR′, —NR—C(NR′R″R′″)═NR″″, —NR—C(NR′R″)═NR′″—S(O)R′, —S(O)₂R′, —S(O)₂NR′R″, —NRSO₂R′, —CN and —NO₂, —R′, —N₃, —CH(Ph)₂, fluoro(C₁₋₄)alkoxo, and fluoro(C₁₋₄)alkyl, in a number ranging from zero to the total number of open valences on aromatic ring system; and where R′, R″, R′″ and R″″ may be independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl and substituted or unsubstituted heteroaryl. When a compound of the disclosure includes more than one R group, for example, each of the R groups is independently selected as are each R′, R″, R′″ and R″″ groups when more than one of these groups is present.

Two of the substituents on adjacent atoms of aryl or heteroaryl ring may optionally form a ring of the formula -T-C(O)—(CRR′)_{q}—U—, wherein T and U are independently —NR—, —O—, —CRR′— or a single bond, and q is an integer of from 0 to 3. Alternatively, two of the substituents on adjacent atoms of aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′— or a single bond, and r is an integer of from 1 to 4.

One of the single bonds of the new ring so formed may optionally be replaced with a double bond. Alternatively, two of the substituents on adjacent atoms of aryl or heteroaryl ring may optionally be replaced with a substituent of the formula —(CRR′)ₛ—X′— (C″R′″)_{d}—, where s and d are independently integers of from 0 to 3, and X′ is —O—, —NR′—, —S—, —S(O)—, —S(O)₂—, or —S(O)₂NR′—. The substituents R, R′, R″ and R″″ may be independently selected from hydrogen, substituted or unsubstituted alkyl, substituted or unsubstituted cycloalkyl, substituted or unsubstituted heterocycloalkyl, substituted or unsubstituted aryl, and substituted or unsubstituted heteroaryl.

As used herein, the term “acyl” refers to an organic acid group wherein the —OH of the carboxyl group has been replaced with another substituent and has the general formula RC(═O)—, wherein R is an alkyl, alkenyl, alkynyl, aryl, carbocylic, heterocyclic, or aromatic heterocyclic group as defined herein). As such, the term “acyl” specifically includes arylacyl groups, such as a 2-(furan-2-yl)acetyl)- and a 2-phenylacetyl group. Specific examples of acyl groups include acetyl and benzoyl. Acyl groups also are intended to include amides, —RC(═O)NR′, esters, —RC(═O)OR′, ketones, —RC(═O)R′, and aldehydes, —RC(═O)H.

The terms “alkoxyl” or “alkoxy” are used interchangeably herein and refer to a saturated (i.e., alkyl-O—) or unsaturated (i.e., alkenyl-O— and alkynyl-O—) group attached to the parent molecular moiety through an oxygen atom, wherein the terms “alkyl,” “alkenyl,” and “alkynyl” are as previously described and can include C₁₋₂₀ inclusive, linear, branched, or cyclic, saturated or unsaturated oxo-hydrocarbon chains, including, for example, methoxyl, ethoxyl, propoxyl, isopropoxyl, n-butoxyl, sec-butoxyl, tert-butoxyl, and n-pentoxyl, neopentoxyl, n-hexoxyl, and the like.

The term “alkoxyalkyl” as used herein refers to an alkyl-O-alkyl ether, for example, a methoxyethyl or an ethoxymethyl group.

“Aryloxyl” refers to an aryl-O— group wherein the aryl group is as previously described, including a substituted aryl. The term “aryloxyl” as used herein can refer to phenyloxyl or hexyloxyl, and alkyl, substituted alkyl, halo, or alkoxyl substituted phenyloxyl or hexyloxyl.

“Aralkyl” refers to an aryl-alkyl-group wherein aryl and alkyl are as previously described, and included substituted aryl and substituted alkyl. Exemplary aralkyl groups include benzyl, phenylethyl, and naphthylmethyl.

“Aralkyloxyl” refers to an aralkyl-O— group wherein the aralkyl group is as previously described. An exemplary aralkyloxyl group is benzyloxyl, i.e., C₆H₅—CH₂—O—. An aralkyloxyl group can optionally be substituted.

“Alkoxycarbonyl” refers to an alkyl-O—C(═O)— group. Exemplary alkoxycarbonyl groups include methoxycarbonyl, ethoxycarbonyl, butyloxycarbonyl, and tert-butyloxycarbonyl.

“Aryloxycarbonyl” refers to an aryl-O—C(═O)— group. Exemplary aryloxycarbonyl groups include phenoxy- and naphthoxy-carbonyl.

“Aralkoxycarbonyl” refers to an aralkyl-O—C(═O)— group. An exemplary aralkoxycarbonyl group is benzyloxycarbonyl.

“Carbamoyl” refers to an amide group of the formula —C(═O)NH₂. “Alkylcarbamoyl” refers to a R′RN—C(═O)— group wherein one of R and R′ is hydrogen and the other of R and R′ is alkyl and/or substituted alkyl as previously described. “Dialkylcarbamoyl” refers to a R′RN—C(═O)— group wherein each of R and R′ is independently alkyl and/or substituted alkyl as previously described.

The term carbonyldioxyl, as used herein, refers to a carbonate group of the formula —O—C(═O)—OR.

“Acyloxyl” refers to an acyl-O— group wherein acyl is as previously described.

The term “amino” refers to the —NH₂ group and also refers to a nitrogen containing group as is known in the art derived from ammonia by the replacement of one or more hydrogen radicals by organic radicals. For example, the terms “acylamino” and “alkylamino” refer to specific N-substituted organic radicals with acyl and alkyl substituent groups respectively.

An “aminoalkyl” as used herein refers to an amino group covalently bound to an alkylene linker. More particularly, the terms alkylamino, dialkylamino, and trialkylamino as used herein refer to one, two, or three, respectively, alkyl groups, as previously defined, attached to the parent molecular moiety through a nitrogen atom. The term alkylamino refers to a group having the structure —NHR′ wherein R′ is an alkyl group, as previously defined; whereas the term dialkylamino refers to a group having the structure —NR′R″, wherein R′ and R″ are each independently selected from the group consisting of alkyl groups. The term trialkylamino refers to a group having the structure —NR′R″R′″, wherein R′, R″, and R′″ are each independently selected from the group consisting of alkyl groups. Additionally, R′, R″, and/or R′″ taken together may optionally be —(CH₂)ₖ— where k is an integer from 2 to 6. Examples include, but are not limited to, methylamino, dimethylamino, ethylamino, diethylamino, diethylaminocarbonyl, methylethylamino, isopropylamino, piperidino, trimethylamino, and propylamino.

The amino group is —NR′R″, wherein R′ and R″ are typically selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

The terms alkylthioether and thioalkoxyl refer to a saturated (i.e., alkyl-S—) or unsaturated (i.e., alkenyl-S— and alkynyl-S—) group attached to the parent molecular moiety through a sulfur atom. Examples of thioalkoxyl moieties include, but are not limited to, methylthio, ethylthio, propylthio, isopropylthio, n-butylthio, and the like.

“Acylamino” refers to an acyl-NH— group wherein acyl is as previously described. “Aroylamino” refers to an aroyl-NH— group wherein aroyl is as previously described.

The term “carbonyl” refers to the —C(═O)— group, and can include an aldehyde group represented by the general formula R—C(═O)H.

The term “carboxyl” refers to the —COOH group. Such groups also are referred to herein as a “carboxylic acid” moiety.

The term “cyano” refers to the group.

The terms “halo,” “halide,” or “halogen” as used herein refer to fluoro, chloro, bromo, and iodo groups. Additionally, terms such as “haloalkyl,” are meant to include monohaloalkyl and polyhaloalkyl. For example, the term “halo(C₁₋₄)alkyl” is mean to include, but not be limited to, trifluoromethyl, 2,2,2-trifluoroethyl, 4-chlorobutyl, 3-bromopropyl, and the like.

The term “hydroxyl” refers to the —OH group.

The term “hydroxyalkyl” refers to an alkyl group substituted with an —OH group.

The term “mercapto” refers to the —SH group.

The term “oxo” as used herein means an oxygen atom that is double bonded to a carbon atom or to another element.

The term “nitro” refers to the —NO₂ group.

The term “thio” refers to a compound described previously herein wherein a carbon or oxygen atom is replaced by a sulfur atom.

The term “sulfate” refers to the —SO₄ group.

The term thiohydroxyl or thiol, as used herein, refers to a group of the formula —SH.

More particularly, the term “sulfide” refers to compound having a group of the formula —SR.

The term “sulfone” refers to compound having a sulfonyl group —S(O₂)R.

The term “sulfoxide” refers to a compound having a sulfinyl group —S(O)R

The term ureido refers to a urea group of the formula —NH—CO—NH₂.

Throughout the specification and claims, a given chemical formula or name shall encompass all tautomers, congeners, and optical- and stereoisomers, as well as racemic mixtures where such isomers and mixtures exist.

Certain compounds of the present disclosure may possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)- or (S)- or, as D- or L- for amino acids, and individual isomers are encompassed within the scope of the present disclosure. The compounds of the present disclosure do not include those which are known in art to be too unstable to synthesize and/or isolate. The present disclosure is meant to include compounds in racemic, scalemic, and optically pure forms. Optically active (R)- and (S)-, or D- and L-isomers may be prepared using chiral synthons or chiral reagents, or resolved using conventional techniques. When the compounds described herein contain olefenic bonds or other centers of geometric asymmetry, and unless specified otherwise, it is intended that the compounds include both E and Z geometric isomers.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the disclosure.

It will be apparent to one skilled in the art that certain compounds of this disclosure may exist in tautomeric forms, all such tautomeric forms of the compounds being within the scope of the disclosure. The term “tautomer,” as used herein, refers to one of two or more structural isomers which exist in equilibrium and which are readily converted from one isomeric form to another.

Unless otherwise stated, structures depicted herein are also meant to include compounds which differ only in the presence of one or more isotopically enriched atoms. For example, compounds having the present structures with the replacement of a hydrogen by a deuterium or tritium, or the replacement of a carbon by ¹³C- or ¹⁴C-enriched carbon are within the scope of this disclosure.

The compounds of the present disclosure may also contain unnatural proportions of atomic isotopes at one or more of atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds of the present disclosure, whether radioactive or not, are encompassed within the scope of the present disclosure.

The compounds of the present disclosure may exist as salts. The present disclosure includes such salts. Examples of applicable salt forms include hydrochlorides, hydrobromides, sulfates, methanesulfonates, nitrates, maleates, acetates, citrates, fumarates, tartrates (e.g. (+)-tartrates, (−)-tartrates or mixtures thereof including racemic mixtures, succinates, benzoates and salts with amino acids such as glutamic acid. These salts may be prepared by methods known to those skilled in art. Also included are base addition salts such as sodium, potassium, calcium, ammonium, organic amino, or magnesium salt, or a similar salt. When compounds of the present disclosure contain relatively basic functionalities, acid addition salts can be obtained by contacting the neutral form of such compounds with a sufficient amount of the desired acid, either neat or in a suitable inert solvent or by ion exchange. Examples of acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like. Certain specific compounds of the present disclosure contain both basic and acidic functionalities that allow the compounds to be converted into either base or acid addition salts.

The neutral forms of the compounds may be regenerated by contacting the salt with a base or acid and isolating the parent compound in the conventional manner. The parent form of the compound differs from the various salt forms in certain physical properties, such as solubility in polar solvents.

Certain compounds of the present disclosure can exist in unsolvated forms as well as solvated forms, including hydrated forms. In general, the solvated forms are equivalent to unsolvated forms and are encompassed within the scope of the present disclosure. Certain compounds of the present disclosure may exist in multiple crystalline or amorphous forms. In general, all physical forms are equivalent for the uses contemplated by the present disclosure and are intended to be within the scope of the present disclosure.

In addition to salt forms, the present disclosure provides compounds, which are in a prodrug form. Prodrugs of the compounds described herein are those compounds that readily undergo chemical changes under physiological conditions to provide the compounds of the present disclosure. Additionally, prodrugs can be converted to the compounds of the present disclosure by chemical or biochemical methods in an ex vivo environment. For example, prodrugs can be slowly converted to the compounds of the present disclosure when placed in a transdermal patch reservoir with a suitable enzyme or chemical reagent.

The term “protecting group” refers to chemical moieties that block some or all reactive moieties of a compound and prevent such moieties from participating in chemical reactions until the protective group is removed, for example, those moieties listed and described in T. W. Greene, P.G.M. Wuts, Protective Groups in Organic Synthesis, 3rd ed. John Wiley & Sons (1999). It may be advantageous, where different protecting groups are employed, that each (different) protective group be removable by a different means. Protective groups that are cleaved under totally disparate reaction conditions allow differential removal of such protecting groups. For example, protective groups can be removed by acid, base, and hydrogenolysis. Groups such as trityl, dimethoxytrityl, acetal and tert-butyldimethylsilyl are acid labile and may be used to protect carboxy and hydroxy reactive moieties in the presence of amino groups protected with Cbz groups, which are removable by hydrogenolysis, and Fmoc groups, which are base labile. Carboxylic acid and hydroxy reactive moieties may be blocked with base labile groups such as, without limitation, methyl, ethyl, and acetyl in the presence of amines blocked with acid labile groups such as tert-butyl carbamate or with carbamates that are both acid and base stable but hydrolytically removable.

Carboxylic acid and hydroxy reactive moieties may also be blocked with hydrolytically removable protective groups such as the benzyl group, while amine groups capable of hydrogen bonding with acids may be blocked with base labile groups such as Fmoc. Carboxylic acid reactive moieties may be blocked with oxidatively-removable protective groups such as 2,4-dimethoxybenzyl, while co-existing amino groups may be blocked with fluoride labile silyl carbamates.

Allyl blocking groups are useful in the presence of acid- and base-protecting groups since the former are stable and can be subsequently removed by metal or pi-acid catalysts. For example, an allyl-blocked carboxylic acid can be deprotected with a palladium(O)-catalyzed reaction in the presence of acid labile t-butyl carbamate or base-labile acetate amine protecting groups. Yet another form of protecting group is a resin to which a compound or intermediate may be attached. As long as the residue is attached to the resin, that functional group is blocked and cannot react. Once released from the resin, the functional group is available to react.

Typical blocking/protecting groups include, but are not limited to the following moieties:

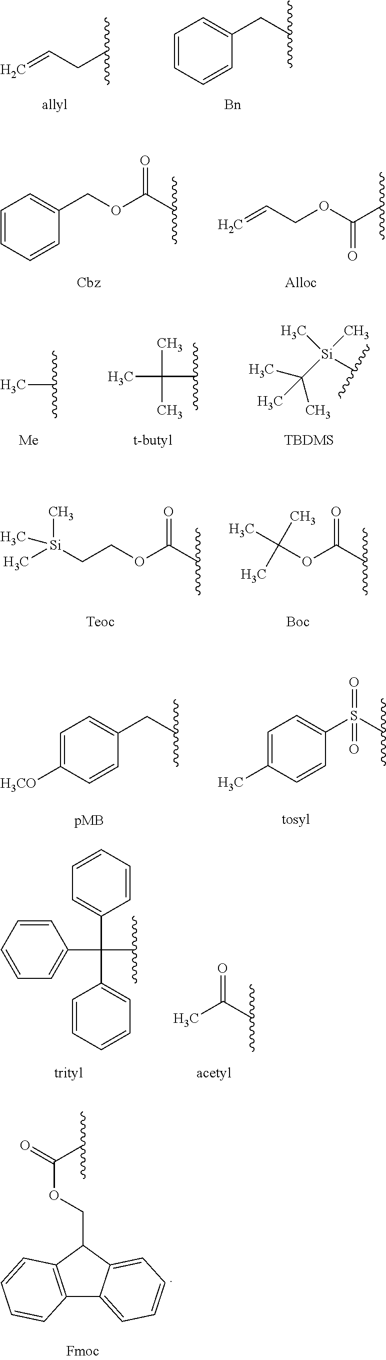

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

For the purposes of this specification and appended claims, unless otherwise indicated, all numbers expressing amounts, sizes, dimensions, proportions, shapes, formulations, parameters, percentages, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments, ±100% in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter. The synthetic descriptions and specific examples that follow are only intended for the purposes of illustration, and are not to be construed as limiting in any manner to make compounds of the disclosure by other methods.

### Example 1

### Maternal Aryl Hydrocarbon Receptor Activation Protects

### Newborns Against Necrotizing Enterocolitis

### 1.1 Abbreviations

NEC (necrotizing enterocolitis), TLR4 (toll like receptor 4), AHR (aryl hydrocarbon receptor, I3C (indole-3-carbinole), CYP1A1 (cytochrome P450 family 1 subfamily A member 1), IL22 (interleukin 22), IL6 (interleukin 6), TNFa (tumor necrosis factor alpha), iNOS (inducible Nitric oxide synthases), RPLPO (ribosomal protein lateral stalk subunit P0), Ecad (E-Cadherin), ILC (innate lymphoid cell), LPL (lamina propria leukocyte), miRNA (microRNA), qRT-PCR (quantitative real-time polymerase chain reaction), DAPI (4′,6-diamidino-2-phenylindole), TUNEL (terminal deoxynucleotidyl transferase dUTP nick end labeling), FITC (fluorescein isothiocyanate), RP (rhodamine phalloidin), ZO-1 (zonula occludens-1), EDTA (ethylenediaminetetraacetic acid), LPS (lipopolysaccharides), WT (wildtype), Ctrl (control), JHDL (Johns Hopkins Drug Library).

### 1.2 Overview

Necrotizing enterocolitis (NEC) is an often-fatal disease of premature infants that is characterized by acute necrosis of the intestine which requires activation of intestinal toll like receptor 4 (TLR4). Current dogma suggests that NEC develops in response to postnatal dietary and bacteriologic exposure, and in utero factors remain largely unexplored. Without wishing to be bound to any one particular theory, it is thought that maternal-fetal signaling plays a role in the development of NEC, and that there exists a window of opportunity in utero to prevent this disease. To test this hypothesis, the presently disclosed subject matter focuses on the aryl hydrocarbon receptor (AHR), a ligand-activated transcription factor that recognizes environmental and dietary ligands to induce immune protection.

To this end, the presently disclosed subject matter, in some embodiments, demonstrates that administration of a diet to pregnant mice that is rich in the AHR ligand indole-3-carbinole (I3C) can prevent NEC in the offspring, and that AHR signaling in the newborn intestinal epithelium prevents NEC by curtailing the extent of TLR4 signaling in the newborn gut. The presently disclosed subject matter also shows that breast milk—a maternal-derived tissue with strong anti-NEC effects—prevents NEC by activating AHR on the newborn gut and reducing TLR4 signaling.

More particularly, NEC protection required AHR activation on the intestinal epithelium to reduce intestinal TLR4, did not require AHR activation on leukocytes (as revealed in Ahr^{ΔIEC}, Ahr^{-/-}, Ahr^{Δlys}) and did not require IL-22 (as revealed in Il22^{-/-} mice). Mice and humans with NEC revealed reduced intestinal AHR, explaining their predisposition to disease development. Finally, using a screen of clinically relevant compounds, an AHR ligand was identified that can activate AHR and limit TLR4 in human tissue, and prevent NEC in mice when administered during pregnancy. Taken together, these findings establish a critical link between maternal-fetal AHR signaling and NEC prevention, offer a novel way to prevent NEC by administering A18 during pregnancy, and highlight a role for AHR in the pathogenesis and treatment of this devastating disease.

### 1.3 Background

Necrotizing enterocolitis (NEC) is a serious disease of premature infants that is characterized by the acute onset of inflammation and necrosis of the intestine, leading eventually to overwhelming sepsis and death. Neu and Walker, 2011; Hackam et al., 2019. NEC develops in the setting of prematurity, formula feeds and bacterial colonization of the newborn gastrointestinal tract, and over a third of affected patients die from the disease. Flahive et al., 2020.

In seeking to understand the pathogenesis of NEC, the inventors' Leaphart et al., 2007; Sodhi et al., 2012; Werts et al., 2019, and others, Jilling et al., 2006; Fawley et al., 2017, have shown that exaggerated signaling of the receptor for bacterial lipopolysaccharide, namely Toll-like receptor 4 (TLR4), on the intestinal epithelium, Sodhi et al., 2012, plays a critical role in NEC development. TLR4 is expressed at higher levels in the premature, as compared with the full-term intestinal epithelium, Sodhi et al., 2012, Gribar et al., 2014, and its activation by luminal bacteria leads to mucosal death and bacterial translocation. Afrazi et al., 2014; Hackam and Sodhi, 2018.

Current dogma suggests that NEC develops in response to dietary and bacteriologic factors that are present in the postnatal period, which explains why all preventive strategies have so far been targeted after birth. Jin et al., 2019; Underwood, 2018. There is emerging evidence to question this dogma, however, and to raise the possibility that NEC also may reflect an in utero process, and by extension, to suggest that interventions in the prenatal period could prevent NEC development. For example, NEC occurs more frequently and with greater severity in babies who are born after in utero bacterial infection, Ogunyemi et al., 2003; Duci et al., 2019, while certain maternal diets have been linked to reduced premature birth, Englund-Ogge et al., 2014; Saunders et al, 2014, which is the biggest risk factor for NEC. Neu and Walker, 2011.

### 1.4 Scope

Based upon these observations, it is now thought that maternal-fetal signaling can modulate the pathogenesis of NEC, and that a window of opportunity may exist in utero to prevent this disease. To test this hypothesis, the presently disclosed subject matter focuses on the aryl hydrocarbon receptor (AHR), a ligand-activated transcription factor that recognizes environmental and dietary ligands, Rothhammer and Quintana, 2019, including those present in green leafy vegetables, Kiss et al., 2011; Amakura et al., 2008, and which has been shown to induce immune protection. Shinde and McGaha, 2018.

The presently disclosed subject matter demonstrates that administration of a maternal diet that is rich in AHR ligand during pregnancy can prevent NEC in the offspring, and that AHR signaling in the newborn intestinal epithelium prevents NEC by curtailing the extent of TLR4 signaling. The presently disclosed subject matter also shows that breast milk—a maternal-derived tissue with strong anti-NEC effects—prevents NEC through activation of AHR in the newborn gut and reduction of TLR4 signaling. Finally, using a screen of clinically relevant compounds, an AHR ligand has been identified that can activate AHR and limit TLR4 in human tissue, thus serving as a novel NEC prevention agent when administered in utero. Taken together, these findings establish a critical link between maternal-fetal AHR signaling and NEC prevention, and highlight a role for AHR in the pathogenesis and treatment of this devastating disease.

### 1.5 Results

### 1.5.1 the Maternal Administration of the AHR Ligand Indole-3-Carbinole (I3C) During Pregnancy Prevents NEC in Newborn Mice

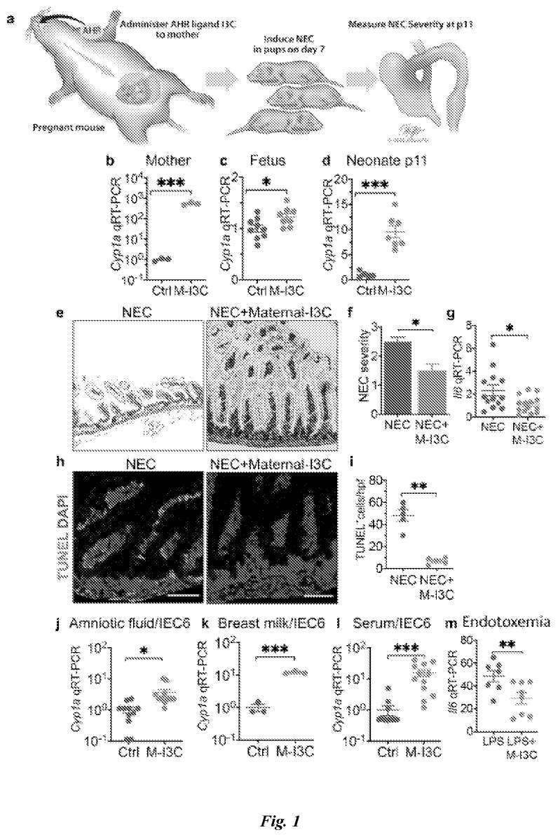

Whether activation of AHR during pregnancy could prevent NEC in newborn mice was initially investigated. The experimental design for this investigation is shown in FIG. 1a. Supplementation of the maternal diet with the AHR ligand indole-3-carbinol (I3C) throughout pregnancy induced the expression of the AHR response gene Cyp1a1 in the small intestine of both the mother (FIG. 1B) and the fetus at e17.5 (FIG. 1c), confirming that AHR activation had occurred in both the mother and fetus. The continuation of administration of I3C to the lactating mother induced Cyp1a1 expression in suckling pups at p11 (FIG. 1d).

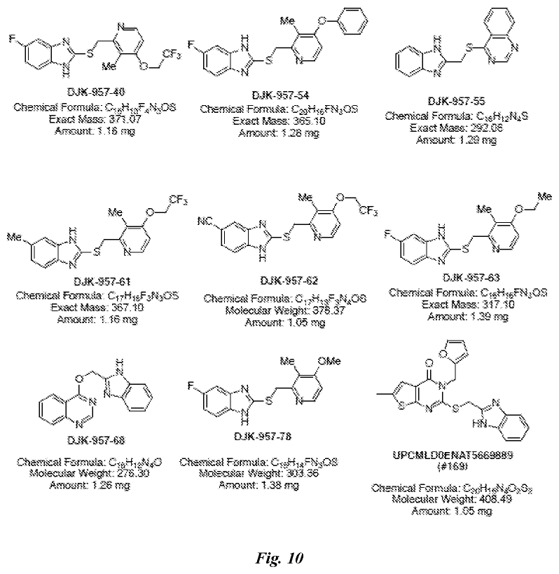

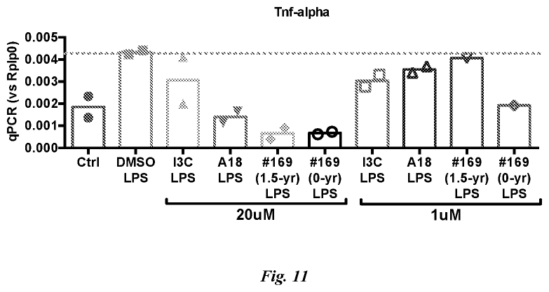

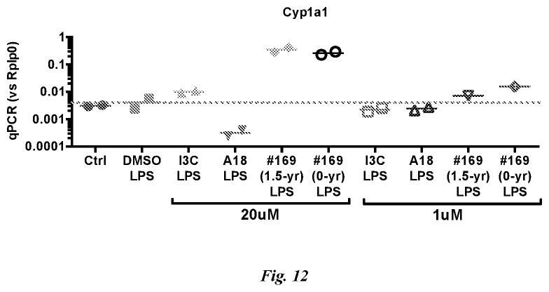

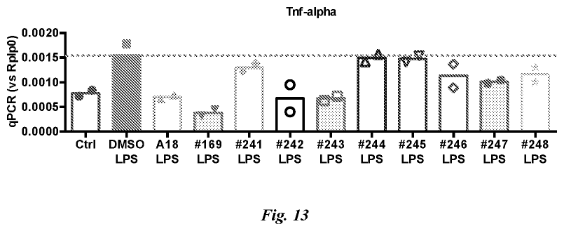

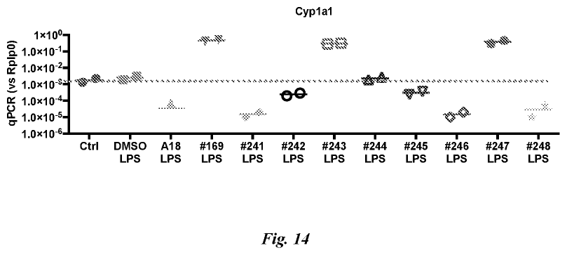

To determine whether maternal AHR administration could prevent NEC, either I3C or a control diet was administered to mice during pregnancy and lactation, and then induced NEC in the pups using a well-validated model, Good et al., 2015; Sodhi et al., 2018, that induces patchy intestinal necrosis, which closely mimics the human disease (see Methods Section 1.7 herein below). As shown in FIG. 1e-FIG. 1i, the oral administration of I3C during pregnancy significantly reduced the severity of experimental NEC in the pups, as manifest by reduced histological injury (FIG. 1e-FIG. 10, reduced expression of 116 in the intestinal epithelium (FIG. 1g), and reduced intestinal epithelial apoptosis (FIG. 1h-FIG. 1i).

Next, to determine whether maternal-derived AHR ligands were present in tissues to which the developing fetus would be exposed, amniotic fluid, breast milk, and serum were harvested from pregnant mice that had been administered either I3C or a control diet, which was then incubated with IEC-6 enterocytes to assess for AHR activation. As shown in FIG. 1j-FIG. 1l, exposure of each of these maternal-derived biological fluids significantly increased the expression of Cyp1a1 in cultured IEC-6 enterocytes, indicating that each maternal derived fluid contains AHR ligands.

Next, whether the maternal administration of I3C could blunt TLR4 signaling in the pup intestine was investigated, given the critical importance of TLR4 signaling to NEC pathogenesis. Leaphart et al., 2007; Sodhi et al., 2012; Hackam et al., 2018; Lu et al., 2014. As shown in FIG. 1m, maternal administration of I3C significantly reduced the induction of the proinflammatory cytokine 116 in the neonatal (p11) mouse intestine in response to the specific TLR4 ligand LPS, as compared to pups from non I3C-treated mothers, supporting a protective effect of maternal AHR activation. Taken together, these findings reveal that maternal delivery of AHR ligands to the fetus can prevent the development of NEC, prompting us to examine AHR signaling in the newborn mice.

1.5.2. The Aryl Hydrocarbon Receptor is Expressed on the Newborn Intestinal Epithelium where its Activation Protects Against the Development of Necrotizing Enterocolitis

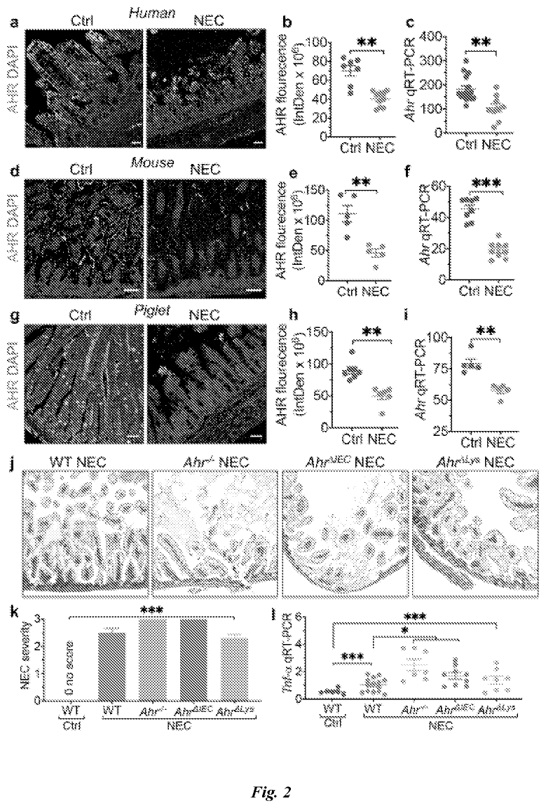

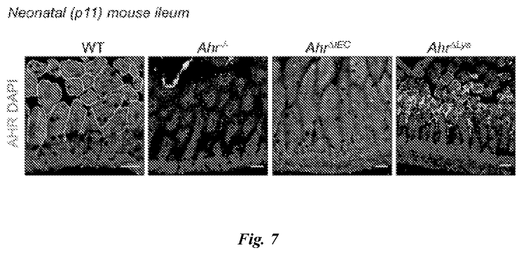

To examine how the administration of AHR ligands during pregnancy can prevent NEC, AHR expression in the intestine in humans, mice and piglets with and without NEC was explored. As shown in FIG. 2, the expression of AHR was significantly reduced in the intestine of infants with NEC as compared with control infants (FIG. 2a-FIG. 2c), and also was reduced in the intestinal mucosa of mice that were induced to develop NEC (FIG. 2d-FIG. 2f), and in an experimental model of NEC in premature piglets (FIG. 2g-FIG. 2i), suggesting a universal response that is not specific for one species. To test whether a lack of AHR signaling was a cause and not a consequence of NEC, NEC was induced in mice from which Ahr globally (Ahr^{-/-}) had been deleted, or specifically from the intestinal epithelium (Ahr^{ΔIEC}) or the myeloid compartment (Ahr^{Δlys}). Confirmation of cell-specific Ahr deletion in each strain is shown in FIG. 7.

As shown in FIG. 2, Ahr^{-/-} mice developed significantly more severe NEC than wild-type mice, as determined by higher histologic grade (FIG. 2j-FIG. 2k) and higher expression of the proinflammatory cytokine gene Tnfa (FIG. 2l). Importantly, AHR signaling on the intestinal epithelium as opposed to leukocytes was required for the protection from NEC in these studies, as Ahr^{ΔIEC} mice had more severe higher grade of NEC compared to wild-type or Ahr^{Δlys} mice (FIG. 2j-FIG. 2l), both of which express AHR on the intestinal epithelium (as seen in FIG. 7).

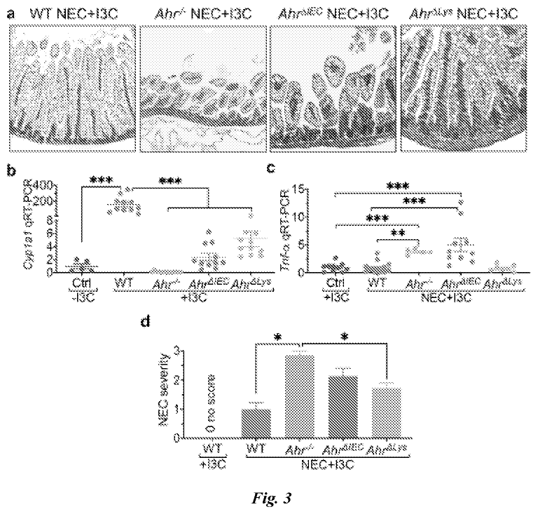

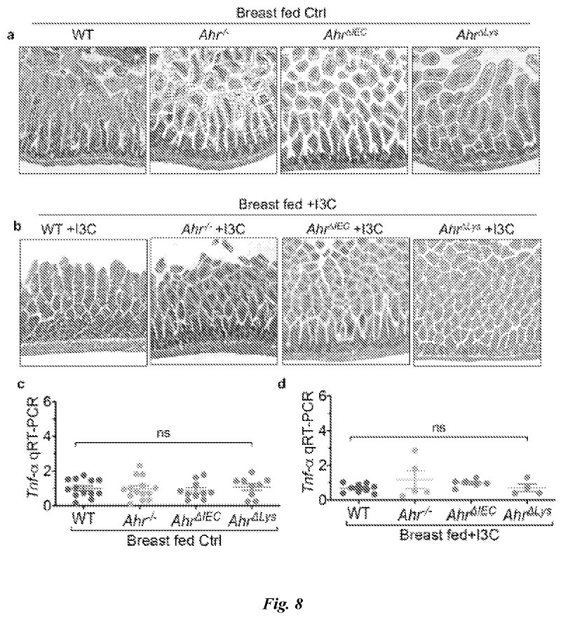

Based on the above findings, whether feeding neonatal mice an AHR ligand could reduce NEC severity was explored. To do so, an AHR-ligand rich infant diet was developed by supplementing the infant formula with I3C, Kiss et al., 2011, which was then administered to pups in our mouse NEC model (FIG. 3a). As shown in FIG. 3b, I3C administration induced the expression Cyp1a1 in the neonatal intestinal epithelium of wildtype and Ahr^{Δlys} mice, but not Ahr^{-/-}, and to a lesser degree in Ahr^{ΔIEC} mice, confirming the appropriate activation of the AHR pathway in the neonatal gut. Importantly, I3C supplementation significantly reduced the severity of NEC in wild-type mice (FIG. 3a, FIG. 3c), but had relatively scant protective effect in either Ahr^{ΔIEC} or no protective effect in Ahr^{-/-} mice (FIG. 3a, FIG. 3c), confirming that AHR activation in the intestinal epithelium by I3C is required for protection against NEC. In an important control, I3C supplementation continued to exert a moderate protective effect when administered to Ahr^{Δlys} mice, which still express AHR on the intestinal epithelium (FIG. 3a, FIG. 3c). For these experiments, the corresponding breast fed controls maintain a normal histology, as shown in FIG. 8.

Taken together, these findings illustrate that AHR activation on the intestinal epithelium protects against NEC development, prompting us to examine the potential mechanisms involved.

### 1.5.3 AHR Activation Protects Against NEC Independent of IL-22 and Intestinal Permeability

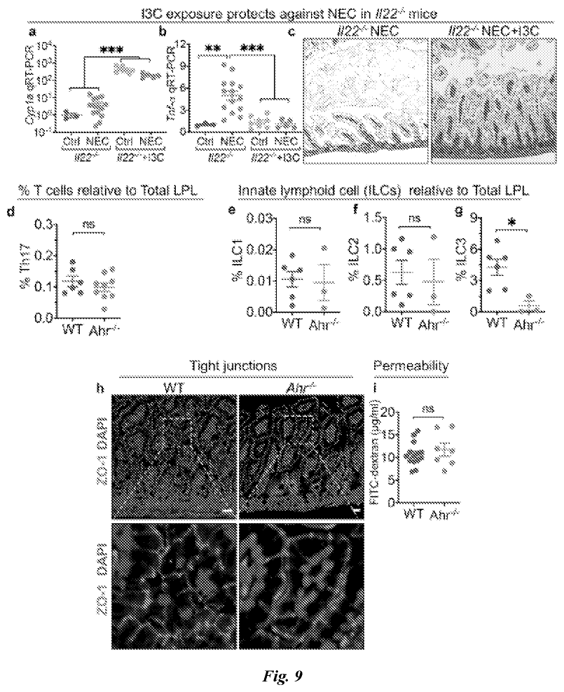

To investigate potential mechanisms by which AHR activation in the neonatal intestinal epithelium can reduce NEC severity, other systems in which AHR activation has been shown to play a protective role were investigated. Specifically, AHR signaling critically regulates the mucosal Th17 and ILC3 lymphocyte populations, each of which are sources of IL-22, Schiering et al., 2017, thus accounting for the essential role of IL-22 in mediating the protective effect of AHR in other models. To investigate whether IL-22 was required for the protection by AHR against NEC as determined in FIG. 2, I3C was administered and then NEC was induced in Il22^{-/-} mice. As shown in FIG. 9, I3C supplementation induced the expression of Cyp1a1 in the intestinal epithelium (FIG. 9a) and reduced NEC severity (FIG. 9b and FIG. 9c), illustrating that IL-22 was not required for the protective effects of AHR activation on NEC. Consistent with this finding, in the early postnatal period in which NEC is induced, it was observed that Th17 (FIG. 9d) and ILCs (FIG. 9e-FIG. 9g) were found to be rare populations in the intestinal mucosa, a finding supported by others. Egan et al., 2016; Chen et al., 2015.

In further consideration of the protective mechanisms by which AHR activation can reduce NEC, the possibility that AHR activation could regulate tight junction expression and intestinal barrier integrity was considered, as has been shown by others in older mice. Singh et al., 2019. No difference was found, however, in the distribution of the tight junction protein ZO-1 in the newborn ileum (FIG. 9h) or intestinal permeability after oral gavage of labelled dextran (FIG. 9i), making it unlikely that the effects of AHR activation can be attributed to barrier integrity. Based upon these studies, and given that TLR4 plays a critical role in NEC pathogenesis, Leaphart et al., 2007; Sodhi et al., 2012; Hackam et al., 2018; Lu et al., 2014, the possibility that AHR activation could reduce TLR4 signaling or expression in the neonatal intestinal epithelium was investigated.

### 1.5.4 AHR Activation Limits TLR4 Signaling and Expression in the Intestinal Epithelium of Mice and Humans

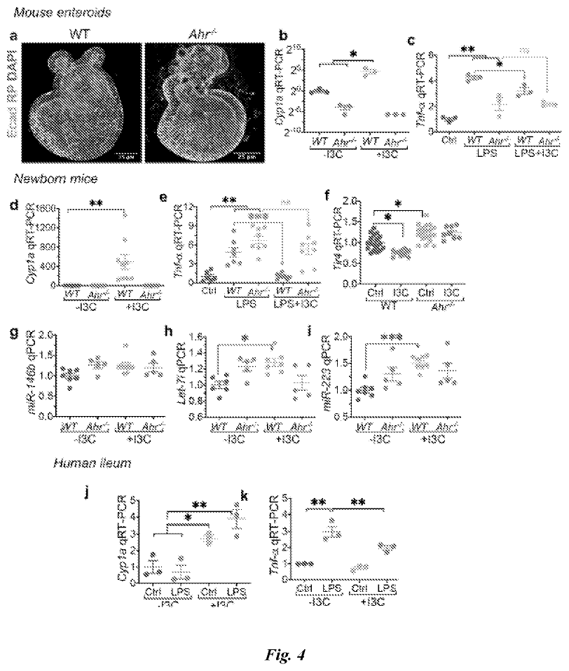

To directly investigate whether AHR activation could limit TLR4 signaling in the intestinal epithelium, studies were first performed in primary enteroids derived from both wild-type and Ahr^{-/-} mice (FIG. 4a). Treatment of enteroids with I3C induced the expression of Cyp1a1 in wild-type, but not Ahr^{-/-} enteroids (FIG. 4b), confirming that AHR is appropriately activated on intestinal epithelial cells.

Importantly, I3C significantly reduced the LPS-mediated induction of Tnfa in wild-type, but not Ahr^{-/-} enteroids (FIG. 4c), consistent with a reduction in TLR4 signaling in intestinal epithelial cells in vitro. In seeking to understand whether I3C could inhibit intestinal Tlr4 in vivo, wild-type p11 mice were fed a diet rich in I3C, which induced Cyp1a1 expression in their intestinal epithelium (FIG. 4d) and significantly reduced TLR4 signaling, as manifest by reduced LPS-induced Tnfa expression (FIG. 4e), which was accompanied by reduced expression of Tlr4 in the intestinal mucosa (FIG. 4f). I3C administration did not induce Cyp1a1 (FIG. 4d) and did not reduce either TLR4 signaling (FIG. 4e) or Tlr4 expression (FIG. 40 in Ahr^{-/-} mice, confirming the specificity of the I3C effect. In gaining insights into how AHR activation could reduce Tlr4 expression, it is noted that I3C significantly upregulated the expression in the ileum of microRNAs that are known to negatively regulate Tlr4 expression, namely miR-146b, miR-223 and let-7i in wild type mice, He et al., 2014, while I3C did not increase the expression of these microRNAs in Ahr^{-/-} mice (FIG. 4g-FIG. 4i).

To assess whether AHR activation could reduce TLR4 signaling in human intestine, we next treated intestinal explant cultures derived from freshly resected intestinal samples from premature infants undergoing surgery for NEC, with both LPS and I3C. As shown in FIG. 4j-FIG. 4k, I3C treatment of human intestinal cultures induced the expression of Cyp1a1, revealing activation of AHR in human tissue, and also reduced TLR4 signaling, as revealed by reduced LPS-induced Tnf-a expression (FIG. 4k). Taken together, these findings indicate that AHR activation inhibits TLR4 signaling and expression in the intestinal epithelium. Given the findings that maternal-fetal signaling can regulate NEC, as well as the observation in FIG. 1 that breast milk can induce the AHR downstream gene Cyp1a1, whether AHR signaling could explain in part the protective effects of breast milk for NEC was then explored.

### 1.5.5 Breast Milk Reduces TLR4 Signaling and Prevents NEC Via AHR Activation in the Newborn Intestinal Epithelium

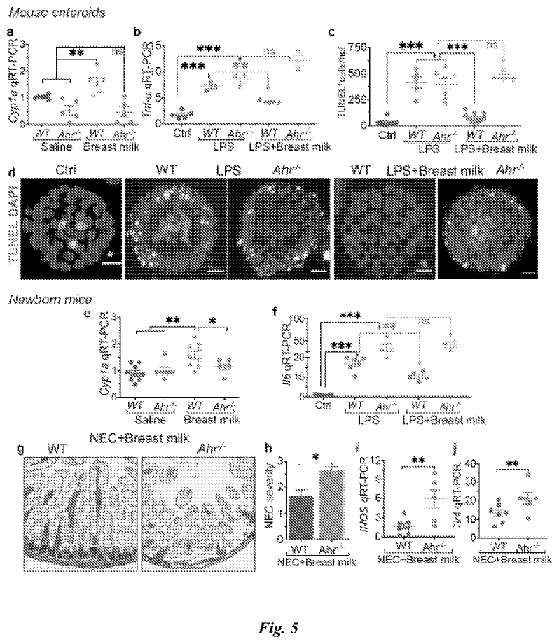

As observed in FIG. 1 that maternal ligands can be delivered in utero to protect NEC in the offspring. These findings were extended to examine whether breast milk—which is by definition rich in maternal-derived ligands—could activate AHR in the intestinal mucosa of the pup, and thus reduce TLR4 signaling and prevent NEC. To test this possibility directly, enteroids were harvested from the ilea of wild-type and Ahr^{-/-} mice, and then treated with LPS in the presence or absence of human breast milk. As shown in FIG. 5a, treatment of wild-type enteroids with breast milk activated AHR, as revealed by induction of Cyp1a1, a finding not seen in either saline-treated or Ahr^{-/-} enteroids, and revealing that breast milk is enriched in maternally-derived AHR ligands.

Importantly, breast milk reduced TLR4 signaling in enteroids in an AHR-dependent manner (FIG. 5b), as indicated by the significant reduction in LPS-induced Tnfa expression in wild-type but not Ahr^{-/-} enteroids after breast milk treatment. TLR4-induced apoptosis of the intestinal epithelium is an important feature of mouse and human NEC, Leaphart et al., 2007; Neal et al., 2012, leading to next assess whether breast milk could prevent TLR4-induced apoptosis in enteroids via AHR. As shown in FIG. 5c-FIG. 5d, LPS significantly increased enterocyte apoptosis in wildtype enteroids, and the degree of induction was reduced after treatment with breast milk, a protective effect that was absent in Ahr^{-/-} enteroids (FIG. 5c-FIG. 5d). Breast milk also required AHR to inhibit TLR4 signaling in the newborn intestinal epithelium, as the oral administration of breast milk to mice that had been maintained without oral feeds for 3 hours induced the expression Cyp1a1 and significantly reduced LPS-induced 116 expression in the intestinal mucosa of wild-type but not Ahr^{-/-} mice (FIG. 5e).

To assess whether the protective effects of breast milk for NEC required AHR, infant formula was supplemented with human breast milk and a reduction in NEC severity in wild-type mice was observed (FIG. 5g), but almost no protection by breast milk on NEC in Ahr^{-/-} mice (FIG. 5h), as revealed by no reduction in histology and severity score (FIG. 5g-FIG. 5h), or the expression of iNos (FIG. 5i), or Tlr4 (FIG. 5j). Taken together, these findings reveal that maternally-derived factors secreted into the breast milk can reduce TLR4 signaling and attenuate NEC in the offspring. Whether these observations could be exploited therapeutically was investigated next, including a search for novel AHR agonists that could prevent NEC after maternal administration.

1.5.6 Discovery of the AHR Ligand, “A18,” which Activates AHR and Reduces TLR4 Signaling in Human Tissue and Prevents NEC in Mice when Administered During Pregnancy

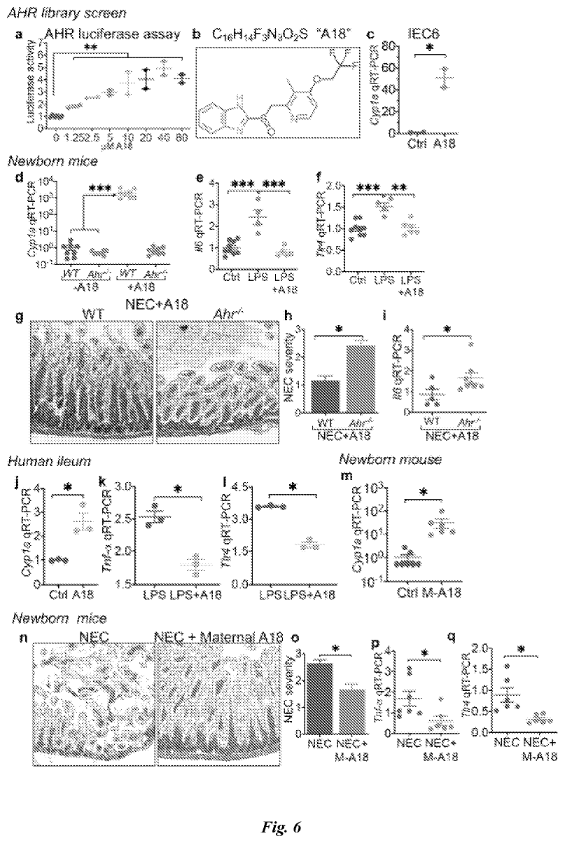

To search for AHR ligands that could be used to prevent or treat NEC when administered during pregnancy, a clinical compound library containing FDA-approved drugs, Chong et al., 2006, was screened against an AHR-luciferase screen in intestinal epithelial cells (IEC-6 cells) (FIG. 6a). The lead compound, shown in FIG. 6b, is a 369KDa molecule with the formula C₁₆H₁₄F₃N₃O₂S, herein called “A18,” which induced the expression of Cyp1a1 in IEC-6 enterocytes (FIG. 6c) and in the intestinal epithelium when fed to wild-type, but not Ahr^{-/-} newborn mice (FIG. 6d) confirming its role as a specific AHR ligand.

Importantly, feeding A18 to mice significantly reduced TLR4-induced 116 expression (FIG. 6e), and reduced Tlr4 expression in the newborn ileum (FIG. 60, providing further confirmation of the ability of AHR to limit TLR4 in the neonatal gut. Further, A18 administration significantly reduced NEC severity when administered orally to wild-type but not Ahr^{-/-} mice, as manifest by improved histology (FIG. 6g), improved NEC severity score (FIG. 6h), and reduced expression of I16 (FIG. 6j). The potential clinical relevance of A18 was found as the ex vivo treatment of human intestine obtained from infants undergoing surgery with A18 significantly induced Cyp1a1 expression (FIG. 6j), confirming its ability to activate AHR in human tissue, and significantly reduced LPS-induced Tnf-a expression (FIG. 6k) and Tlr4 expression (FIG. 6l), consistent with the findings in mice (FIG. 6e).

Finally, to assess the ability of A18 to modulate the maternal-fetal signaling pathway and reduce NEC, we administered A18 to mice during pregnancy. As shown in FIG. 6m, the administration of A18 during pregnancy induced the expression of Cyp1a1 in the offspring pup intestine, and also significantly reduced NEC severity as manifest by reduced Tnf-a (FIG. 6n) and Tlr4 expression (FIG. 6o) in the neonatal gut, and improved histology and NEC severity (FIG. 6p-q). Taken together, these results suggest a surprising role for maternal-fetal AHR signaling in NEC, and raise the possibility that A18 can serve as a novel agent that can be administered either during pregnancy or postnatally for the prevention and treatment of this devastating disease.

### 1.6 Discussion

The persistently high mortality of necrotizing enterocolitis, Han et al., 2020, reveals both a lack of a sufficient understanding of its pathogenesis, and an urgency to approach the disease differently. This study sheds light on the possibility that NEC arises from reduced AHR signaling in the intestinal epithelium of the premature infant and shows that AHR ligands may be passed from mother to infant—both during pregnancy via the fetal circulation, and in the postnatal period through the breast milk—where they can attenuate the severity of this disease. The mechanism by which AHR activation in the intestinal epithelium attenuates NEC severity involves a reduction in signaling and expression of the innate immune receptor TLR4, whose expression is elevated in the premature bowel as compared with the full term bowel, Gribar et al., 2014; Sodhi et al., 2012, and whose activation on the intestinal epithelium has been shown to be critical for NEC development. Leaphart et al., 2007; Sodhi et al., 2012; Egan et al., 2016. The therapeutic potential of the current findings was revealed by the identification of the AHR ligand A18 to prevent NEC when fed to mice during pregnancy, and by showing its ability to reduce TLR4 signaling in human bowel ex vivo. In view of the fact that NEC almost always develops in the absence of breast milk and the presence of formula feeds, Nino et el, 2016, these findings suggest the possibility that the development of NEC may reflect impaired AHR signaling in the neonatal intestine, and also show that strategies to enhance the delivery of AHR ligands either directly to the neonate, or secondarily through the mother, may offer new strategies for the prevention or treatment of NEC.

Previous authors have shown AHR to be a gatekeeper for the availability of AHR ligands in the intestinal immune system, Schiering et al. 2017, from either microbial or dietary origin, Brawner et al., 2019; Gao et al., 2018, and a critical link between AHR, dietary factors and intestinal immunity has been proposed. Shinde and McGaha, 2018. The current studies now reveal that AHR signaling on leukocytes is dispensable for the protective role of AHR against NEC, in favor of AHR signaling on the intestinal epithelium. This observation is distinct from a large body of work that reveals that the activation of AHR on leukocytes is required for the maintenance of Th17 and ILC3 cells in the intestinal mucosa, Kiss et al., 2011; Veldhoen et al., 2008, leading to AHR-mediated protection from colitis through IL-22 release. Monteleone et al., 2011.

Differences between those studies and our own may lie in the fact that Th17 and ILC3 cells are rare in the neonatal intestinal mucosa at the time points in which NEC develops. Chen et al., 2015; Egan et al., 2016. It is noteworthy that intestinal AHR signaling also has been linked to the release of antimicrobial peptides, Kiss et al., 2011, and to increased differentiation of secretory cells, Alvarado et al, 2019, processes, which are both downstream of TLR4 activation in the gut, Sodhi et al., 2012; Wu et al., 2014, and thus consistent with the current work. Without wishing to be bound to any one particular theory, it is thought that that differences between the current work and prior studies also reveal unique features of NEC, including differences in the location of disease along the gastrointestinal tract (NEC is an ileal disease while colitis affects the colon), age (NEC occurs in the newborn period whereas these other models occur in older mice), as well as differences in microbial ligands which may be present in models of colitis versus NEC, and which could activate AHR.

One of the most translationally relevant findings of the current study is the identification of an AHR ligand that can prevent NEC, either when offered orally during pregnancy (FIG. 6m-q), or when administered directly to the neonatal pups (FIG. 6g-i). As revealed from its chemical structure in FIG. 6b, the AHR ligand A18 belongs to the benzimidazole class of proton pump inhibitors, which also includes omeprazole. Yang et al., 2016. These FDA-approved drugs are currently in clinical use for hyperacidity diseases, and could be conceivably repurposed for NEC, just as they have been repositioned as anticancer therapeutics. Jin et al., 2014. The identification of the heterocylic A18 as a potent AHR substrate reflects the broad range of hydrophobic structural motifs that bind to the AHR. Gutierrez-Vazquez and Quintana, 2018.

In seeking to understand the chemical mechanism of AHR binding so as to develop novel analogues for clinical use, it is noted that the indole nucleus is a privileged ligand for AHR, Denison and Faber, 2017, suggesting that the benzimidazole core of A18 is the main pharmacophore for AHR activation. Major limits of A18 for clinical use in preventing NEC include the fact that while A18 is rapidly absorbed and approximately 97% bound in human plasma, it is extensively metabolized by CYP3A4 and CYP2C18, and plasma elimination half-life is only approximately 2 h in humans, Landes et al., 1995, leaving much room for further improvement. Moreover, acid reduction therapy has been linked to an increase in NEC when administered to premature neonates, Alganabi et al., 2019, indicating that A18 will need to be modified in order to enhance its AHR signaling but reduce its effects on gastric acidity.

The current findings may have direct impact on clinical medicine. First, these finding have shown that NEC may not only be a disease of the postnatal period, but may also reflect impaired maternal-fetal signaling through AHR. Second, these finding also have provided important insights into how breast milk may protect against NEC, by revealing its ability to activate AHR on the neonatal intestinal epithelium, where it can attenuate TLR4 signaling. But perhaps of most significance is the opportunity to interfere with the molecular pathways that lead to the development of NEC through the intra-partum oral delivery of AHR ligands including I3C or A18. If confirmed in clinical studies, these findings may offer the unique ability to intervene in the setting of premature labor, by administering an AHR ligand that could serve to protect the gut and reduce the risk of NEC development in the neonate. In support of the possible success of a strategy in which antenatal delivery of AHR ligands may protect the neonate, it is noteworthy that pregnant women who adhered to a Mediterranean diet—which is rich in AHR ligands—were found to have significantly less necrotizing enterocolitis than mothers who did not adhere to a Mediterranean diet. Parlapani et al., 2019. Taken in aggregate, the current finds offer the prospect of a renewed approach to NEC, and perhaps finally to a chance to alter the trajectory of this devastating disease.

### 1.7 Methods

### 1.7.1 Mice

All mice were purchased from the Jackson Laboratory. To generate tissue specific knockouts of Ahr from either epithelial cells (Ahr^{ΔIEC}) or leukocytes (Ahr^{Δlys}), mice harboring a floxed allele of Ahr^{fx} (Ahrtm3.1Bra/J) were bred with transgenic mice expressing Villin-Cre (B6.Cg-Tg(Vill-cre)997Gum/J) and LysM-Cre (B6.129P2-Lyz2tm1(cre)Ifo/J) respectively. To generate global knockouts of Ahr (Ahr^{-/-}), Ahr mice were bred with transgenic mice expressing global Cre under the transcriptional control of a human cytomegalovirus minimal promoter CMV-Cre (B6.C-Tg(CMVcre) 1Cgn/J). The homozygous I122c″ mice (C57BL/6-Il22tm1.1(icre)Stck/J), in which the presence of iCre abolishes expression of 1122, served as Il22^{-/-} mice. Ahlfors et al., 2014. For flow cytometry experiments, Foxp3^{GFP} mice or Rorgt^{GFP} mice were bred with Ahr to generate Foxp3^{GFP}; Ahr^{-/-} and Rorgt^{GFP}; Ahr reporter mice, respectively. All mice were housed in a specific pathogen free environment on a 12-hour-light/12-hour-dark cycle with free access to water and standard rodent chow (Teklad global 18% protein rodent diets, Envigo) except otherwise specified. All mouse and piglet experiments were approved by the Johns Hopkins University Animal Care and Use Committee.

### 1.7.2 Discovery of AHR Ligands for the Prevention or Treatment of NEC in Mice

The intestinal epithelial cells line IEC-6 (ATCC CRL-1592) was stably transduced with lentiviral particles containing Cignal Lenti XRE Reporter (luc) (Qiagen). Cells were treated serially with individual chemicals contained within the Johns Hopkins Drug Library (JHDL), which contains a series of FDA-approved drugs, Chong et al., 2006, (kindly provided by Dr. Jun O. Liu, Johns Hopkins University) at 10 μM for 24 hours, and the luciferase activity was quantified using the SpectraMax M3 (Molecular Devices).

Hits were validated for activation of AHR signaling by oral gavage into neonatal C57/B16 mice (p11), and measuring the expression of the AHR activation reporter Cyp1a1 by qRT-PCR in the intestinal mucosa 24 hours later. The lead compound from these studies is herein labelled “A18”.

### 1.7.3 Induction of NEC in Mice.

Experimental NEC was induced in 7-day old mice as we have previously described, Good et al., 2015; Sodhi et al., 2018; Egan et al., 2016, by gavage feeding newborn mice with formula containing Similac Advance infant formula (Abbott Nutrition): Esbilac (PetAg) canine milk replacer, 2:1 ratio, which was supplemented with enteric bacteria made from a stock created from a specimen obtained from an infant with surgical NEC five times per day.

Additionally, the mice were subjected to hypoxia (5% 02-95% N₂) for 10 min in a hypoxia chamber (Billups-Rothenberg) twice daily for 4 days. The AHR ligands I3C and A18 were administered by oral gavage during the induction of NEC at the dose of 25 mg/kg body weight/day and 300 mg/kg body weight/day, respectively. To test the protective effect of breast milk on AHR signaling a NEC, human breast milk (Innovative Research) was supplemented to the formula at the final concentration of 5%.

Age-matched breast milk-fed mouse pups were used as healthy controls. Evaluation of ileal histology and expression of pro-inflammatory cytokines by qRT-PCR at a fixed point in the terminal ileum 2-cm proximal to the cecum, were used to determine the disease severity.

### 1.7.4 Induction of NEC in Piglets

To induce NEC in piglets, timed-pregnant White Yorkshire (Yorkshire x Landrace) sows were obtained from Oak Hill Genetics, and piglets were delivered prematurely via cesarean section at approximately 95% gestation as we have described, Good et al, 2014, in a modification of the work of Sun et al., 2018. NEC was induced in piglets by gavage formula at 15 mL/kg every 3 h (120 ml/kg per day) for 4 days (n=7) of the following (per liter): Pepdite Junior (93.9 g; Nutricia), MCT Oil (38.3 g, USP grade; Now Foods), whey protein isolate (56 g, Now Foods), and 837 g water, which was supplemented with enteric bacteria made from a specimen obtained from an infant with surgical NEC.

### 1.7.5 Mice Endotoxemia Model

Endotoxemia was induced in neonatal mouse pups (p11) by administering 5 mg/kg lipopolysaccharides (LPS) via intraperitoneal injection, and ileal samples were harvested 6 hours after LPS treatments. I3C or A18 were given through oral gavage daily 3 days prior to the LPS injection at the dose of 25 mg/kg/day and 300 mg/kg/day, respectively.

### 1.7.6 AHR Induction in Pregnant Mice

For activation of AHR during pregnancy in mice, wild type mice were fed an AHR ligand-free diet, Brawner et al., 2019, (AIN-76A, Bio Serv), and the AHR ligands I3C and A18 were administered by oral gavage at the dose of 25 mg/kg/day and 300 mg/kg/day, respectively. I3C and A18 were administered to the pregnant mother daily until the offspring were studied further for experimental NEC or endotoxemia as above.

1.7.7 Harvest and Culture of Enteroids from Mouse Ileum

Primary intestinal crypt cultures (enteroids) were generated from the ileum of neonatal (p7-p11) wild type and Ahr′ mice at as described, Neal et al., 2012, and maintained in Matrigel (Corning). The enteroids were digested and passed using TrypLE Express (Gibco) weekly, and used between passage 3 and 10 for all experiments. The enteroids were pre-treated with I3C (200 mM, 12 h), A18 (20 μM, 12 h) or human breast milk (100 μL/mL, Innovative Research, prepared by 5-minute-cenrifugation at 12,000 g and then filtration of supernatant through a 0.22-μm filter) and then treated with LPS (50 μg/mL) for 4 hours for further analysis.

### 1.7.8 Human Ileal Sample Collection and Explant Culture

De-identified human ileal samples were collected via waiver of consent from the Office of Human Subjects Research Review Boards at Johns Hopkins University (IRB00094036), during surgery for NEC or at the time of stoma closure. For RNA isolation and qRT-PCR analysis, fresh samples were snapfrozen in liquid nitrogen immediately. For human explant culture, fresh ileum samples from NEC patients or patient undergoing stoma re-anastomosis were washed with sterile phosphate-buffered saline containing gentamycin (5 μg/mL), minced into 2- to 4-mm diameter pieces, and then cultured in Dulbecco's modified Eagle growth medium supplemented with 10% fetal bovine serum, 4 μg/mL human recombinant insulin and 100 μg/mL Primocin. Human ilea explant cultures were then pre-treated with 200 μM I3C or 20 μM A18 for 15 minutes, and then with 50 μg/mL LPS for 6 hours, then processed for total RNA isolation followed by qRT-PCR.

### 1.7.9 Immunofluorescence Staining

5-μm tissue sections from mouse, piglet and human intestine were rehydrated, heated in 10-mmol/L citric acid buffer for antigen retrieval, permeabilized with 0.1% Tween-20, probed with primary antibodies overnight at 4° C., probed with secondary antibody and 4′,6-diamidino-2-phenylindole (DAPI, Biolegend) for 1 hour at room temperature, and then mounted in Gelvatol mounting media (Sigma-Aldrich) for imaging. To assess apoptosis, samples were incubated with terminal deoxynucleotidyl transferase dUTP nick end labeling (TUNEL) detection solution (In Situ Cell Death Detection Kit, Roche) as per manufacturer's instructions. Slides were incubated with the nuclear marker DAPI, mounted using Gelvatol solution prior to imaging using a Nikon Eclipse Ti Confocal microscope. For immunofluorescence staining of enteroids, cells were cultured in 10 μL Matrigel on chamber slides.

After 20 minutes fixation in 4% PFA at room temperature, the enteroids were washed with PBS, probed with primary antibodies overnight at 4° C., probed with secondary antibody and DAPI for 1 hour at room temperature, and then mounted in Gelvatol mounting media for imaging. Antibodies are: goat-anti AHR (sc-8089, Santa Cruz), rabbit-anti KI67 (ab15580, Abcam), Anti-Ecadherin (AF748; R&D Systems), Rhodamine Phalloidin (R415; Thermo Fisher Scientific).

1.7.10 RNA Isolation, cDNA Synthesis, Quantification of mRNA and miRNA.

Total RNA was isolated using the RNeasy mini kit (Qiagen) and complementary DNA was synthesized from 0.5 μg RNA using QuantiTect Reverse Transcription kit (Qiagen) following the manufacturer's protocols. The mRNA quantification was performed on the Bio-Rad CFX96 Real-Time System (Bio-Rad) using iTaq™ universal SYBR® Green supermix (Bio-Rad) and the primers listed in Table 1. The relative mRNA expression levels were normalized against the expression of the housekeeping gene ribosomal protein lateral stalk subunit P0 (Rplp0). For microRNA isolation, freshly harvested, snap-frozen ileal tissue was subjected to total RNA isolation using the RNeasy plus universal mini kit (Qiagen) and miRNA was quantified using miScript PCR starter kit (Qiagen) following the manufacturer's protocols. The relative miRNA expression levels were normalized against the expression of housekeeping miRNA miR-191.

TABLE 1
Primers.

| | | | |
|---|---|---|---|
| | | | Amplicon |
| Gene | Forward primer | Reverse primer | size (bp) |

Mouse sequences

| | | | |
|---|---|---|---|
| Ahr | ATGTCCATGTATCAGTGCCAG | CTGCTCAAGTCGGACGAATAG | 149 |
| IL6 | CCAATTTCCAATGCTCTCCT | ACCACAGTGAGGAATGTCCA | 182 |
| IL1 [symbol] | AGTGTGGATCCCAAGCAATACCCA | TGTCCTGACCACTGTTGTTTCCCA | 175 |
| Rnf43 | GTGATGCTGCGAAGCTGATG | CTGAAGAGGATCCGGTCTGC | 120 |
| Rplp0 | GGCGACCTGGAAGTCCAACT | CCATCAGCACCACAGCCTTC | 143 |
| Tlr4 | TTTATTCAGAGCCGTTGGTG | CAGAGGATTGTCCTCCCATT | 186 |
| Tnf [symbol] | TTCCGAATTCACTGGAGCCTCGAA | TGCACCTCAGGGAAGAATCTGGAA | 144 |

Human sequences

| | | | |
|---|---|---|---|
| Ahr | GTCCAGTCTAATGCACGCCT | ATGGCAGGAAAAGGGTTGGT | 182 |
| CYP1A1 | AATTTCGGGGAGGTGGTTGG | GATGTGGCCCTTCTCAAAGGT | 368 |
| Rplp0 | GGCGACCTGGAAGTCCAACT | CCATCAGCACCACAGCCTTC | 143 |
| Tnf [symbol] | GGCGTGGAGCTGAGAGATAAC | GGTGTGGGTGAGGAGCACAT | 120 |

Pig sequences

| | | | |
|---|---|---|---|
| Ahr | CCACTTCAGCCACCATCCAT | ATGCACAGCTCTGCTTCAGT | 140 |
| Rplp0 | GGCGACCTGGAAGTCCAACT | CCATCAGCACCACAGCCTTC | 143 |

Rat sequences

| | | | |
|---|---|---|---|
| Cyp1a1 | TCCTGGAGACCTTCCGACAT | AACCTGCCACTGGTTCACAA | 128 |
| Rplp0 | GGCGACCTGGAAGTCCAACT | CCATCAGCACCACAGCCTTC | 143 |

micro RNA sequences

| | |
|---|---|
| Let-7i | TGAGGTAGTAGTTTGTGCTGTT |
| miR-146b | TGAGAACTGAATTCCATAGGCT |
| miR-223 | TGTCAGTTTGTCAAATACCCCA |
| miR-191 (housekeeping) | CAACGGAATCCCAAAAGCAGCTG |

Genotyping sequence

| | |
|---|---|
| Ahr [symbol] | GTCACTCAGCATTACACTTTCTA, GGTACAAGTGCACATGCCTGC (Knockout: 180 bp) |
| Ahr [symbol] | CAGTGGGAATAAGGCAAGAGTGA, GGTACAAGTGCACATGCCTGC |
| | (Wild type: 1068 bp, loxP: 140 bp) |
| Cre | GTTCGCAAGAACCTGATGGACA, CTAGAGCCTGTTTTGCACGTTC (Transgene: 339 bp) |
| Foxp3 [symbol] | CACCTATGCCACCCTTATCC, ATTGTGGGTCAAGGG GAAG (Wild type: 341 bp), |
| | mutant: 390 bp) |
| IL22 [symbol] | CAGGCTCTCCTCTCAGTTATCA, TCCTGAAGGCCAAAATAGG, CCTCAGGTTCAGCAG |
| | GGAAC (Wild type: 424 bp, mutant: 313 bp) |
| ROR [symbol] | CCCCCTGCCCAGAAACACT, GGATGCCCCCATTCACTTACTTCT, CGGACACGCTGA |
| | ACTTGTGG (Wild type: 174 bp, mutant: 241 bp) |

[symbol]

indicates data missing or illegible when filed

### 1.7.11 Lamina Propria Cell Isolation and Flow Cytometry

Lamina propria cells were isolated from the newborn mouse ileum according to the methods of Hepworth et al., 2013. In brief, the mesentery was removed from the freshly isolated ileum, and the bowel was then opened longitudinally and cut into 1-cm pieces, and incubated in PBS containing 5% fetal bovine serum, 1-mM dithioerythritol (Sigma-Aldrich) and 1 mM EDTA at 37° C. for 20 minutes with agitation at 180 rpm. After filtration through a 70-μm cell strainer, the remaining tissue was finely minced with scissors, and incubated in RPMI containing 2% fetal bovine serum, 0.5 mg/mL collagenase/dispase (Sigma-Aldrich), and 0.02 mg/mL DNase (Sigma-Aldrich) at 37° C. for 40 minutes with agitation at 180 rpm. After filtration through sequential 70-μm and 40-μm cell strainers, the lamina propria leukocytes were collected between the interface of 40% and 60% discontinuous Percoll in preparation for flow cytometry. Single-cell suspensions were washed using ice-cold FACS buffer (PBS, 1% BSA, 0.01% NaN₃) and incubated with anti-CD16/CD32 (BD Bioscience) to block Fc receptor binding (20 minutes, 4° C.) on mouse cells. Cells were pelleted by centrifugation and resuspended in optimal concentrations of fluorochrome-conjugated antibodies in ice-cold FACS buffer to stain surface molecules. The dead cells were stained using Fixable Viability Violet Dye (Thermo Fisher) in PBS.

Intracellular staining was performed using the Foxp3 buffer set (Biosciences). After washing, the samples were analyzed on a BD LSRII flow cytometer for ILCs or BD Accuri™ C6 Plus flow cytometer for Th17 cells. Data analysis was performed using FlowJo software. The fluorochrome-conjugated antibodies used in this study include: anti-mouse CD90.2 Alexa FluorR 700 (30-H12, BioLegend), antimouse CD3e PerCP-Cyanine5.5 (145-2C11, eBioscience), anti-mouse CD5 PerCP-Cyanine5.5 (53-7.3, eBioscience), anti-mouse CD45R (B220) PerCP-Cyanine5.5 (RA3-6B2, eBioscience), anti-mouse CD11c PerCP-Cyanine5.5 (N-418, eBioscience), anti-mouse CD11b PerCP-Cyanine5.5 (M1/70, eBioscience), anti-mouse Gata-3 PE-Cyanine7 (TWAJ, eBioscience), anti-mouse T-bet eFluorR 660 (4B10, eBioscience), anti-mouse ROR gamma (t) PE (B2D, eBioscience), anti-mouse EOMES Alexa FluorR 488 (Dan11mag, eBioscience), and anti-mouse CD4 APC (RM4-5, BD Biosciences).

### 1.7.12 Intestinal Permeability Assay

Pups were gavaged with 500 mg/kg of fluorescein isothiocyanate (FITC)— conjugated dextran (4 kDa, Sigma-Aldrich). Blood was collected from the orbital sinus under isofluorane anesthesia 3 h later, and the serum fluorescence was measured using the SpectraMax M3 spectrophotometer (Molecular Devices).

### Statistical Analysis.

Data were analyzed for statistical significance by t test, one-way ANOVA using GraphPad Prism (GraphPad 8). Graphs show individual dots for each mouse or human sample. A p value of less than 0.05 was considered statistically significant, and data are presented as mean±SEM as indicated.

### Example 2

### Discovery of Novel Aryl Hydrocarbon Receptor Ligands and their Analogues for the Prevention and Treatment of Inflammatory Disorders

### 2.1 Overview

The current technology seeks to prevent or treat inflammatory conditions that affect children, most notably necrotizing enterocolitis (NEC), by activating the aryl hydrocarbon receptor (AHR).

Necrotizing enterocolitis (NEC) is a serious disease of premature infants that is characterized by the acute onset of inflammation and necrosis of the intestine, leading eventually to overwhelming sepsis and death. NEC develops in the setting of prematurity, formula feeds and bacterial colonization of the newborn gastrointestinal tract, and over a third of affected patients die from the disease. In seeking to understand the pathogenesis of NEC, we and others have shown that exaggerated signaling of the receptor for bacterial lipopolysaccharide, namely Toll-like receptor 4 (TLR4), on the intestinal epithelium plays a critical role in NEC development. TLR4 is expressed at higher levels in the premature as compared with the full-term intestinal epithelium, and its activation by luminal bacteria leads to mucosal death and bacterial translocation. The Hackam group has discovered that activation of the aryl hydrocarbon receptor (AHR) in mice and human tissue can inhibit TLR4 signaling, and thus serve to dampen inflammation in the gut.

The finding that AHR activation could inhibit TLR4 signaling therefore led us to search for novel AHR agonists that could be used as therapeutics for NEC. To this end, Hackam and coworkers identified the AHR analogue “A18”, which had been identified and sold commercially as the proton pump inhibitor “Lansoprazole”. The administration of lansoprazole effectively prevented and treated NEC in mouse models, and dampened inflammation in the intestine of human resected at the time of surgery for NEC. Analogues of A18 were then synthesized and developed. A total of 235 analogues were tested in mouse enteroid cell lines, and a “hit”, referred to herein as compound 169, was identified that activated AHR, and significantly inhibited TLR4 signaling in the gut.

The presently disclosed subject matter identifies compound #169 as an AHR agonist with the ability to inhibit TLR4 in the gut, and, pending in vivo analysis, to thus serve as a novel agent for the prevention and treatment of NEC. A computational similarity search based on A18 was conducted in an in-house library. Hundreds of structures were initially identified, binned, and selected for further investigation. Subsequent screens identified analogs, such as compound 169, as well as other structures referred to as #243 and #247. Subsequently, an SAR studies was conducted and representative compounds disclosed herein were prepared.

Importantly, it has been shown that A18 can be administered to pregnant mothers (mice) to treat NEC in the premature pups. This observation suggests that #169, and other identified analogs, could potentially be administered as a gut protective agent for the fetus when administered to the mother in the event of premature labor.

### 2.2 General Considerations

Unless stated to the contrary, where applicable, the following considerations apply: All reactions were performed under an N₂ atmosphere that had been passed through a column (10×2 cm) of Drierite. All glassware and stir bars were dried in an oven for at least 3 h prior to use. Reactions run in microwave vials were sealed with Teflon 20-mm septa and crimped to ensure atmosphere exclusion. When necessary, degassed solvents were prepared by sparging with N₂ for 1 h. Reactions were monitored by TLC analysis (pre-coated silica gel 60 F254) and spots were visualized (UV lamp 254 nm and 365 nm). Purifications by chromatography were performed with SiO₂ (230-400 mesh) on a Teledyne ISCO system with reusable cartridges and monitored at 254 and 280 nm. Reverse phase chromatography was performed on a Biotage Isolera system with C18-SiO₂ cartridges and monitored 200-400 nm. ¹H/¹³C NMR spectra were recorded on Bruker Avance, 300/75 MHz, Bruker Avance 400/100 MHz or Bruker Avance 500/125 MHz instruments.

High resolution mass spectra were obtained on a Micromass UK Limited, Q-TOF Ultima API or a Thermo Scientific Exactive Orbitrap LC-MS. Chemical shifts were reported in parts per million (ppm) with the residual solvent peak (CDCl₃: 7.26 ppm for 1H, 77.16 ppm for ¹³C; DMSO-d₆: 2.50 ppm for 1H, 39.52 ppm for ¹³C) used as the internal standard. Chemical shifts were tabulated as follows: chemical shift, multiplicity (s=singlet, d=doublet, t=triplet, q=quartet, dd=doublet of doublet, dt=doublet of triplet, ddd=doublet of doublet of doublet, m=multiplet, brs=broad singlet), coupling constant(s), and integration. Known intermediates were assigned based on comparison with authentic ¹H NMR spectra. IR spectra were obtained using neat samples on a Perkin-Elmer 100 IR-ATR spectrometer. Melting points were obtained using a Mel-Temp instrument and are uncorrected.

### 2.3 Synthesis

### 2.3.1 Pyridine-Imidazole Series

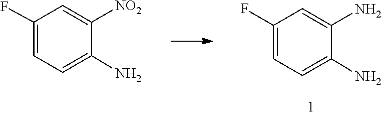 [SMILES: Nc1ccc(F)cc1[N+](=O)[O-] ; Nc1ccc(F)cc1N]

### Preparation of 5-Fluoro-1,2-diaminobenzene (1)

To a 50 mL round bottom flask was added 2-nitro-5-fluoroaniline (1.56 g, 10 mmol) and ethanol 20 mL. Anhydrous SnCl₂ (9.47 g, 50 mmol, 5 equiv.) as a solid. The flask was equipped with a condenser and placed in an 80° C. sand bath under N₂ and the mixture was refluxed for 4 h. The reaction was poured into a sep. funnel, diluted with 80 mL dichloromethane and then treated with 100 mL 2M NaOH. The resulting emulsion was filtered through Celite to remove all the tin, and the resulting biphasic mixture was separated in a sep. funnel. The aq. phase was washed with dichloromethane (50 mL) and the combined organic phases were dried with brine (30 mL), dried over MgSO₄, filtered and concentrated to afford a yellow solid. 1.21 g, 95%. ¹H NMR (300 MHz, Chloroform-d) δ 6.64 (dd, J=8.4, 5.5 Hz, 1H), 6.46 (dd, J=9.9, 2.8 Hz, 1H), 6.39 (td, J=8.5, 2.8 Hz, 1H), 3.35 (s, 4H). Spectral data are in agreement with the literature. Chen et al., 2018.

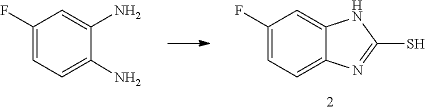 [SMILES: Nc1ccc(F)cc1N ; Fc1ccc2nc(S)[nH]c2c1]

### Preparation of 5-fluoro-2-thiobenzimidazole (2)

To a 10 mL microwave vial was added 5-fluoro-orthophenylenediamine (0.63 g, 5 mmol), potassium ethyl xanthogenate (0.96 g, 6 mmol, 1.2 equiv) and 5 mL of ethanol. To this mixture was added 2.5 mL water and the mixture was sonicated for 10 min until only a fine dispersion remained. The vial was capped and crimped and the reaction was then placed in an 80° C. bath and heated for 16 h. The vial was then allowed to cool to ambient temperature and water (15 mL) was added. A white solid precipitated. The pH of the solution was adjusted to ca. 5, the solution was filtered and the residue was washed with water (2×30 mL). The residue was then dried under airflow to afford a tan solid. 330 mg, 40%. ¹H NMR (500 MHz, DMSO-d₆) δ 12.63 (d, J=16.2 Hz, 2H), 7.12 (dd, J=9.4, 4.5 Hz, 1H), 7.01-6.91 (m, 2H). Spectral data are in agreement with the literature. Liu et al., 2018.

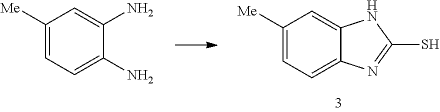 [SMILES: Cc1ccc(N)c(N)c1 ; Cc1ccc2nc(S)[nH]c2c1]

### Preparation of 5-methyl-2-thiobenzimidazole (3)

To a 10 mL microwave vial was added 5-methyl-1,2-diaminobenzene (181 mg, 1.5 mmol) and potassium ethyl xanthogenate (285 mg, 1.8 mmol, 1.2 equiv). 2 mL ethanol was added, followed by 1 mL water and the mixture was sonicated for 10 min until only a fine powder remained. The vial was capped and crimped and the reaction was then placed in an 80° C. bath and heated for 16 h. The vial was then allowed to cool to room temperature and water (15 mL) was added. A white solid precipitated. The pH of the solution was adjusted to ca. 5 (some solid dissolved) and the solution was filtered and the residue was washed with water (2×30 mL). The residue was then dried under airflow to afford a white solid. 198 mg, 81%. 1H NMR (300 MHz, DMSO-d₆) δ 12.78-11.90 (br s, 2H), 7.08-6.99 (m, 1H), 6.94 (d, J=8.1 Hz, 2H), 2.34 (s, 3H). Spectral data are in agreement with the literature. Mavrova et al., 2015.

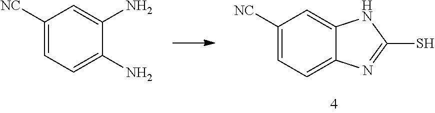 [SMILES: N#Cc1ccc(N)c(N)c1 ; N#Cc1ccc2nc(S)[nH]c2c1]

### Preparation of 5-cyano-2-thiobenzimidazole (4)

To a 10 mL microwave vial was added 5-cyano-1,2-diaminobenzene (200 mg, 1.5 mmol) and potassium ethyl xanthogenate (285 mg, 1.8 mmol, 1.2 equiv). 2 mL ethanol was added, followed by 1 mL water and the mixture was sonicated for 10 min until only a fine powder remained. The vial was capped and crimped and the reaction was then placed in an 80° C. bath and heated for 16 h. The vial was then allowed to cool to room temperature and water (15 mL) was added. A yellow-brown solid precipitated. The residue was filtered and then the filter cake was dissolved with 1 N NaOH and collected via suction. To this solution was added NH₄C₁ until pH<7. The precipitated material was collected by filtration to afford a white solid. 111 mg 43%. ¹H NMR (300 MHz, DMSO-d₆) δ 12.97 (s, 2H), 7.61-7.48 (m, 2H), 7.34-7.23 (m, 1H). Spectral data are in agreement with the literature. Liu et al., 2018.

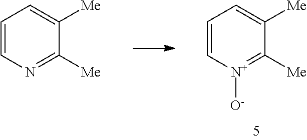 [SMILES: Cc1cccnc1C ; Cc1ccc[n+]([O-])c1C]

### Preparation of 2, 3-dimethylpyridine N-oxide (5)

To a 100 mL round-bottom flask was added 2,3-lutidine (2.14 g, 20 mmol), CHCl₃ (40 mL), followed by mCPBA (77%, 5.74 g, 25 mmol, 1.25 equiv). The reaction was then heated to 60° C. for 18 h. After the reaction was complete, the mixture was poured into a sep. funnel and washed with 15 mL sat. aq. sodium thiosulfate. The phases were separated and the organic phase was washed with brine, dried over MgSO₄, filtered and collected to afford a white solid. 1.93 g, 78%. 1H NMR (400 MHz, Chloroform-d) δ 8.22 (d, J=6.4 Hz, 1H), 7.12 (d, J=7.8 Hz, 1H), 7.06 (t, J=7.1 Hz, 1H), 2.55 (s, 3H), 2.38 (s, 3H). Spectral data are in agreement with the literature. Palav et al., 2019.

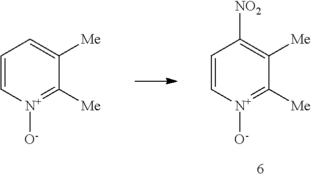 [SMILES: Cc1ccc[n+]([O-])c1C ; Cc1c([N+](=O)[O-])cc[n+]([O-])c1C]

### Preparation of 2,3-dimethyl-4-nitropyridine N-oxide (6)

To a 100 mL round bottom-flask containing 5 (1.93 g, 15.6 mmol) was added sulfuric acid (20 mL). Under stirring, nitric acid (20 mL) was added slowly, and the mixture was then heated to 120° C. overnight. The mixture was poured unto crushed ice, neutralized with excess sat. aq. Na₂CO₃ to adjust pH to 10 and the product was extracted with ethyl acetate (3×50 mL). The combined organic fractions were dried over MgSO₄, filtered, concentrated to afford a white solid. 1.13 g, 43%. ¹H NMR (300 MHz, Chloroform-d) δ 8.23 (d, J=7.2 Hz, 1H), 7.73 (d, J=7.3 Hz, 1H), 2.61 (s, 3H), 2.59 (s, 3H). Zhang and Duan, 2011.

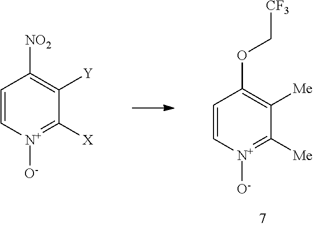

### Preparation of 2,3-dimethyl-4-trifluoroethoxypyridine N-oxide (7)

To a 10 mL microwave vial was added 6 (0.336 g, 2 mmol), followed by trifluoroethanol (4 mL). K₂CO₃ (0.414 g, 1.5 mmol) was added as a solid, the reaction was purged with N₂ for 10 min and then stirred at 80° C. for 16 h. The reaction was allowed to cool to room temperature, transferred to a sep. funnel and diluted with 10 mL water and 15 mL dichloromethane. The aq. phase was extracted with a further 15 mL dichloromethane, the organic phases were combined, washed with 10 mL brine, dried over MgSO₄, filtered and concentrated. The residue was purified by SiO₂ (10 g cartridge, 0 to 10% MeOH in dichloromethane) to afford the desired product as a white solid. 236 mg, 53%: ¹H NMR (300 MHz, Chloroform-d) δ 8.23 (d, J=7.3 Hz, 1H), 6.66 (d, J=7.3 Hz, 1H), 4.42 (q, J=7.8 Hz, 2H), 2.58 (s, 3H), 2.28 (s, 3H). ¹⁹F NMR (282 MHz, Chloroform-d) δ-73.82 (t, J=7.7 Hz). Spectral data are in agreement with the literature. Kubo et al., 1990.

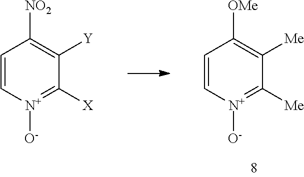

### Preparation of 2,3-dimethyl-4-methoxypyridine N-oxide (8)

To a 10 mL-microwave vial was added 7 (168 mg, 1 mmol) and K₂CO₃ (414 mg, 3 mmol, 3 equiv). MeOH (2 mL) was then added to the reaction. The vial was sealed and heated to 100° C. (bath temp) for 6 h. 50 μL mesitylene (369 μmop was added as an internal standard and product yield was assayed by NMR. (92% yield). The mixtures were allowed to cool to room temperature and then was diluted with water/dichloromethane (20 mL each) and the phases were separated. The aq. phase was washed with a further 20 mL dichloromethane, the organic phases were combined and washed with brine (10 mL). The organic phase was then dried over MgSO₄, filtered and concentrated. Purification by SiO₂ (10 g cartridge, 0 to 10% MeOH in dichloromethane) afforded the desired product as a white solid. 155 mg, 51% over two experiments. ¹H NMR (300 MHz, Chloroform-d) δ 8.18 (d, J=7.2 Hz, 1H), 6.65 (d, J=7.2 Hz, 1H), 3.89 (s, 3H), 2.56 (s, 2H), 2.21 (s, 3H). Spectral data are in agreement with the literature. International PCT Patent Application Publication No. WO2010/117425 for Certain Substituted Pyrimidines, Pharmaceutical Compositions Thereof, and Methods for Their Use, to Shi et al., published Oct. 14, 2010.

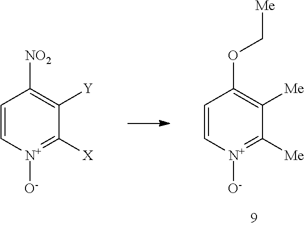

### Preparation of 2,3-dimethyl-4-ethoxypyridine N-oxide (9)

To a 10 mL-microwave vial was added 6 (168 mg, 1 mmol) and K₂CO₃ (414 mg, 3 mmol, 3 equiv). Ethanol (200 proof) was added to the reaction without further purification. The vial was sealed and heated to 120° C. (bath temp) for 12 h. The product was extracted with dichloromethane/H₂O (20 mL each). The aq. phase was washed with a further 20 mL dichloromethane, the organic phases were combined and washed with 10 mL brine. The organic phase was dried over MgSO₄ filtered and concentrated. Purification by SiO₂ (10 g cartridge, 0% to 10% MeOH in dichloromethane) afforded a white solid. 85 mg, 51%. ¹H NMR (300 MHz, Chloroform-d) δ 8.15 (d, J=7.2 Hz, 1H), 6.62 (d, J=7.2 Hz, 1H), 4.08 (q, J=7.0 Hz, 2H), 2.56 (s, 3H), 2.22 (s, 3H), 1.48 (t, J=7.0 Hz, 3H). Spectral data are in agreement with the literature. Kuehler et al., 1995.

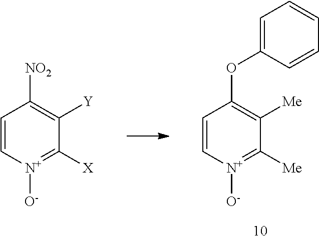

### Preparation of 2,3-dimethyl-4-phenoxypyridine N-oxide (10)

To a 10 mL microwave vial was added phenol (638 mg, 6.8 mmol, 5 equiv), DMF (3 mL), and K₂CO₃ (750 mg, 5.4 mmol, 4 equiv). The mixture was stirred well and 6 (228 mg, 1.35 mmol) was added as a solid. The vial was capped and the mixture heated to 140° C. for 18 h. After the reaction was complete the material was transferred to a sep. funnel and diluted with 30 ml dichloromethane and 20 ml water. The phases were separated and the aqueous phase was extracted with a further 30 mL dichloromethane. The organic phase was washed with brine 20 mL, dried over MgSO₄, filtered and concentrated. The product was purified by SiO₂ (10 g cartridge, 0 to 10% MeOH in dichloromethane) to afford a white solid. 235 mg, 81%. 1H NMR (500 MHz, Chloroform-d) δ 8.09 (d, J=7.2 Hz, 1H), 7.42 (dd, J=8.5, 7.4 Hz, 2H), 7.23 (t, J=7.4 Hz, 1H), 7.03 (dd, J=7.5, 1.6 Hz, 2H), 6.56 (d, J=7.2 Hz, 1H), 2.61 (s, 3H), 2.34 (s, 3H).

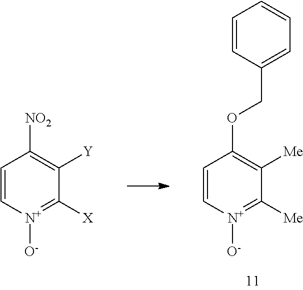

### Preparation of 2,3-dimethyl-4-benzyloxypyridine N-oxide (11)

To a 10 mL microwave vial was added 6 (228 mg, 1.35 mmol) then benzyl alcohol (3 mL). The material was sonicated until homogenous and K₂CO₃ (750 mg, 5.4 mmol, 4 equiv.) was added as a solid. The vial was capped and the mixture heated to 140° C. for 18 h. After the reaction was complete (TLC) the material was transferred to a sep. funnel and diluted with 30 ml dichloromethane and 20 ml water. The phases were separated and the aqueous phase was extracted with a further 30 mL dichloromethane. The organic phase was washed with brine 20 mL, dried over MgSO₄, filtered and concentrated. The product was purified by SiO₂ (10 g cartridge, 0 to 10% MeOH in dichloromethane) to afford a white solid. 212 mg, 68%. 1H NMR (300 MHz, Chloroform-d) δ 8.15 (d, J=7.2 Hz, 1H), 7.42 (s, 4H), 6.70 (d, J=7.3 Hz, 1H), 5.13 (s, 2H), 2.57 (s, 3H), 2.27 (s, 3H). Spectral data are in agreement with the literature. Kohler et al., 1998.

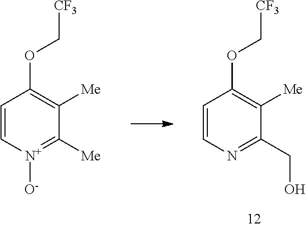 [SMILES: Cc1c(OCC(F)(F)F)cc[n+]([O-])c1C ; Cc1c(OCC(F)(F)F)ccnc1CO]

### Preparation of 2-methylenehydroxy-3-methyl-4-trifluoroethoxypyridine (12)

To 7 (0.213 g, 0.8 mmol) in a 50-mL round bottom flask was added 4 mL MeOH followed by 4 mL 1M NaOH. The reaction was stirred for 18 h at ambient temperature. The mixture was then poured into a sep. funnel, diluted with 10 mL dichloromethane and 10 mL water. Then sat. aq. NaHCO₃ (2 mL) was added and the phases were separated. The aq. phase was extracted with 10 mL dichloromethane, the organic phases were combined, washed with brine (10 mL), dried over MgSO₄, filtered and concentrated. Purification by SiO₂ (10 g cartridge, 10% to 100% EtOAc in hexanes) afforded product as a white fluffy powder. 162 mg, 91%. 1H NMR (500 MHz, Chloroform-d) δ 8.40 (d, J=5.7 Hz, 1H), 6.74 (d, J=3.9 Hz, 1H), 4.73 (s, 2H), 4.46 (q, J=7.9 Hz, 2H), 2.14 (s, 3H). Spectral data are in agreement with the literature. Kubo et al., 1990.

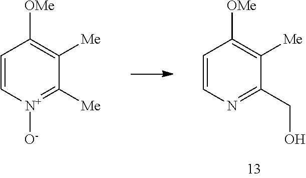 [SMILES: COc1cc[n+]([O-])c(C)c1C ; COc1ccnc(CO)c1C]

### Preparation of 2-methylenehydroxy-3-methyl-4-methoxypyridine (13)

To a microwave vial was added 8 (155 mg, 1 mmol), and 1 mL dichloromethane followed by 0.7 mL TFAA. The vial was capped and then heated at 50° C. bath temp for 48 h. The mixture was then added dropwise to 5 mL MeOH and then hydrolyzed by first adding 5 mL 1M NaOH and then basifying to pH 14 with solid NaOH. The mixture was stirred for 3 h at ambient temperature, then transferred to a sep. funnel. The mixture was diluted with water/dichloromethane (20 mL each) and the phases were separated. The aq. phase was washed with 20 mL dichloromethane, the organic phases were combined and washed with brine (10 mL), dried over MgSO₄ filtered and concentrated to afford a white solid. Product was sufficiently pure for further reactions. 125 mg, 81%. 1H NMR (500 MHz, Chloroform-d) δ 8.34 (d, J=5.7 Hz, 1H), 6.76 (d, J=5.7 Hz, 1H), 4.68 (s, 2H), 3.91 (s, 3H), 2.06 (s, 3H). Spectral data are in agreement with the literature. Kohler et al., 1998.

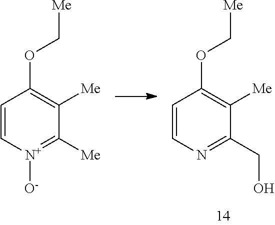 [SMILES: CCOc1cc[n+]([O-])c(C)c1C ; CCOc1ccnc(CO)c1C]

### Preparation of 2-methylenehydroxy-3-methyl-4-ethoxypyridine (14)

To a microwave vial was added 9 (84 mg, 0.5 mmol) and 1 mL dichloromethane followed by 0.7 mL TFAA. The vial was capped and then heated at 50° C. bath temp for 48 h. The mixture was then added dropwise to methanol (5 mL) 1N NaOH (5 mL) was added and the mixture basified to pH 14 with solid NaOH. The mixture was stirred for 3 h at ambient temperature, then transferred to a sep. funnel. The mixture was diluted with water/dichloromethane (20 mL each) and the phases were separated. The aq. phase was washed with 20 mL dichloromethane, the organic phases were combined and washed with brine (10 mL), dried over MgSO₄ filtered and concentrated to afford a white solid. Product was sufficiently pure for further transformations. 63 mg, 75%. ¹H NMR (300 MHz, Chloroform-d) δ 8.22 (d, J=5.7 Hz, 1H), 6.63 (d, J=5.7 Hz, 1H), 4.58 (s, 2H), 4.03 (q, J=7.0 Hz, 3H), 1.97 (s, 3H), 1.39 (t, J=7.0 Hz, 3H). Spectral data are in agreement with the literature. Kuehler et al., 1995.

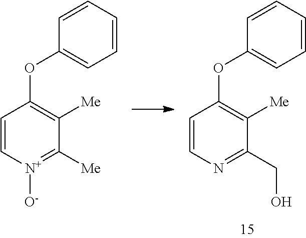 [SMILES: Cc1c(Oc2ccccc2)cc[n+]([O-])c1C ; Cc1c(Oc2ccccc2)ccnc1CO]

### Preparation of 2-methylenehydroxy-3-methyl-4-phenoxypyridine (15)

To a 10 mL microwave vial was added 10 (235 mg, 1.1 mmol) dissolved in 1.5 mL of dichloromethane. To this was added TFAA (1 mL) and the reaction vessel was sealed and heated to 40° C. for 3 hrs. The vessel was allowed to cool and then MeOH was added dropwise until no further reaction was observed. The mixture was transferred to a 4-dram vial and then MeOH (5 mL) was added and the pH was adjusted to >12 with 2N NaOH. This mixture was allowed to stir for 3 h. The resulting mixture was acidified with sat. aq. NaHCO₃ to approximately pH<10 and then the aqueous phase was extracted twice with 30 mL dichloromethane each. The product was purified by SiO₂ (10 g cartridge, 0% to 50% ethyl acetate in dichloromethane) to afford a white solid. 153 mg, 65% 1H NMR (300 MHz, Chloroform-d) δ 8.26 (d, J=5.7 Hz, 1H), 7.51-7.37 (m, 2H), 7.27-7.20 (m, 1H), 7.13-7.00 (m, 2H), 6.58 (d, =5.6 Hz, 1H), 4.75 (s, 2H), 2.21 (s, 3H). Spectral data are in agreement with the literature. International PCT Patent Application No. WO2001/34578 to Carcanague et al., for Compounds with Anti-Helicobacter Pylori Activity, published May 17, 2001.

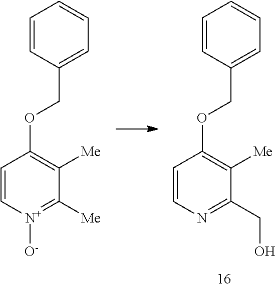 [SMILES: Cc1c(OCc2ccccc2)cc[n+]([O-])c1C ; Cc1c(OCc2ccccc2)ccnc1CO]

### Preparation of 2-methylenehydroxy-3-methyl-4-benzyloxypyridine (16)

To a 10 mL microwave vial was added 11 (212 mg, 0.92 mmol) dissolved in 1.5 mL of dichloromethane. To this was added TFAA (1 mL) and the reaction vessel was sealed and heated to 40° C. for 3 hrs. The vessel was allowed to cool and then MeOH was added dropwise until no further reaction was observed. The mixture was transferred to a 4-dram vial and then MeOH (5 mL) was added and the pH was adjusted to >12 with 2N NaOH. This mixture was allowed to stir 3 h. The resulting mixture was acidified with sat. aq. NaHCO₃ to about pH 10 and then the aqueous phase was extracted twice with 30 mL dichloromethane each. The product was purified by SiO₂ (10 g cartridge, 0% to 50% ethyl acetate in dichloromethane) to afford a white solid. 83 mg, 39%. 1H NMR (300 MHz, Chloroform-d) δ 8.32 (d, J=5.7 Hz, 1H), 7.52-7.32 (m, 5H), 6.80 (d, J=5.7 Hz, 1H), 5.17 (s, 2H), 4.70 (s, 2H), 2.13 (s, 3H). Spectral data are in agreement with the literature. Kühler et al., 1998.

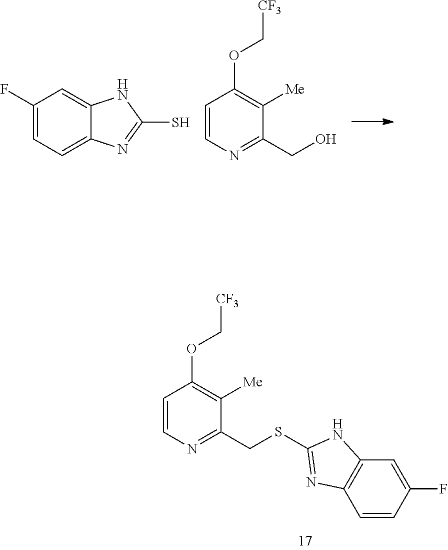

### Preparation of 5-fluoro-2-(((3-methyl-4-(2,2,2-trifluoroethoxy)pyridin-2-yl)methyl)thio)-1H-benzo[d]imidazole (17)

To a conical microwave vial was added 12 (75 mg, 0.34 mmol, 1.1 equiv). Dichloromethane (0.7 mL) was added, followed by SOCl₂ (36 μL, 0.5 mmol, 1.5 equiv). A further 0.7 mL dichloromethane was added and the mixture was heated to 40° C. for 1 h with stirring. The mixture was then transferred to a 4-dram vial and water (0.2 mL) was added. Dichloromethane was removed by rotary evaporation. The residue was then taken up in methanol (0.7 mL) and the 5-fluoro-2-thiobenzimidazole (0.052 g, 0.31 mmol) was added as a solid. The pH was adjusted to 11 with 1N NaOH and the mixture stirred for a further 90 min. The mixture was then poured into a sep. funnel, diluted with 3 mL water and extracted with 10 mL dichloromethane. The aq. phase was washed with a further 10 mL dichloromethane, the organic phases were combined, dried over MgSO₄, filtered and concentrated. Purification by SiO₂ (10 g cartridge, 0% to 3% MeOH in dichloromethane) afforded a mixture of product and starting material. Follow up column in (10 g cartridge, 10% to 50% ethyl acetate in hexanes in 5% gradient increments) cleanly separated starting material and product (72 mg) but was insufficient for submission. Final purification (10 g cartridge, 0% to 40% ethyl acetate in dichloromethane) afforded a yellow solid. 72 mg, 63%. mp. 168-169° C. 1H NMR (400 MHz, Chloroform-d) δ 12.77 (s, 1H), 8.35 (d, J=5.7 Hz, 1H), 7.37 (dd, J=8.8, 4.7 Hz, 1H), 7.15 (dd, J=9.0, 2.5 Hz, 1H), 6.87 (ddd, J=9.7, 8.8, 2.5 Hz, 1H), 6.68 (d, J=5.8 Hz, 1H), 4.37 (q, J=7.8 Hz, 2H), 4.33 (s, 2H), 2.26 (s, 3H). 13C NMR (126 MHz, Chloroform-d) δ 162.58, 160.16, 158.27, 157.62, 152.28, 147.35, 122.77 (q, J=277.9 Hz), 121.86, 109.96 (d, J=25.4 Hz), 106.06, 65.55 (q, J=36.5 Hz), 34.68, 10.69. ¹⁹F NMR (376 MHz, Chloroform-d) δ-73.73 (t, J=7.8 Hz), −121.04. HRMS M+H⁺ calcd. 372.07882, found 372.07709. IR 2922.0, 1586.8, 1479.2, 1454.7, 1435.4, 1347.1, 1312.8, 1285.1, 1258.4, 1158.1, 1113.2, 977.5, 913.8, 859.9, 841.3, 800.9, 762.7, 738.6, 662.4.

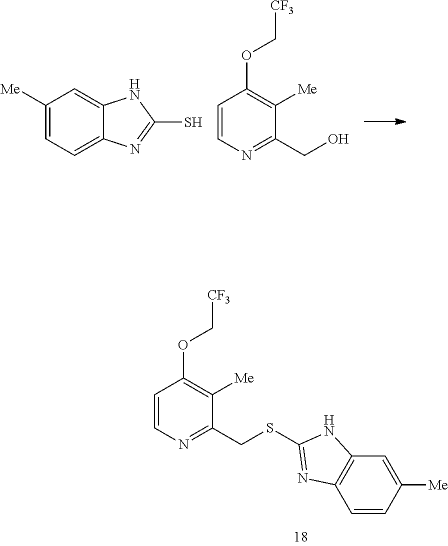

### Preparation of 5-methyl-2-(((3-methyl-4-(2,2,2-trifluoroethoxy)pyridin-2-yl)methyl)thio)-1H-benzo[d]imidazole (18)

To a conical microwave vial was added 12 (30 mg, 0.14 mmol). 0.7 mL of dichloromethane was added, followed by SOCl₂ (15 μL, 0.2 mmol, 1.5 equiv). The mixture turned into a thick white paste. A further 0.7 mL of dichloromethane was added and the mixture was heated to 40° C. for 1 h with stirring. The mixture was then transferred to a 4-dram vial and water (0.2 mL) was added. Dichloromethane was removed by rotary evaporation. The residue was then taken up in methanol (0.7 mL) and the 5-fluorothiobenzimidazole (22 mg, 0.14 mmol, 1 equiv). The pH was adjusted to 11 with 1N NaOH and the mixture stirred for a further 90 min. The mixture was then poured into a sep. funnel, diluted with 3 mL water and extracted with 10 mL dichloromethane. The aq. phase was washed with a further 10 mL dichloromethane, the organic phases were combined, dried over MgSO₄, filtered and concentrated. Purification by SiO₂ (10 g cartridge, 20 to 50% ethyl acetate in dichloromethane) afforded a white solid. 33 mg, 66%. mp. 153-155° C. 1H NMR (300 MHz, Chloroform-d) δ 8.33 (d, J=5.7 Hz, 1H), 7.35 (d, J=8.2 Hz, 1H), 7.25 (s, 1H), 6.94 (dd, J=8.2, 1.6 Hz, 1H), 6.64 (d, J=5.8 Hz, 1H), 4.35 (q, J=7.8 Hz, 2H), 4.33 (s, 2H), 2.38 (s, 3H), 2.24 (s, 3H). 13C NMR (76 MHz, Chloroform-d) δ 162.43, 157.85, 150.58, 147.44, 131.71, 124.64, 123.35, 121.72, 105.95, 65.52 (d, J=36.6 Hz), 34.90, 21.62, 10.68. ¹⁹F NMR (282 MHz, Chloroform-d) δ-73.76. HRMS M+H⁺ calcd. 368.10389, found 368.10296. IR, 2924.1, 1583.7, 1475.0, 1444.1, 1255.0, 1159.3, 1110.4, 968.1, 912.4, 857.0, 836.6, 795.9, 739.0, 662.6.

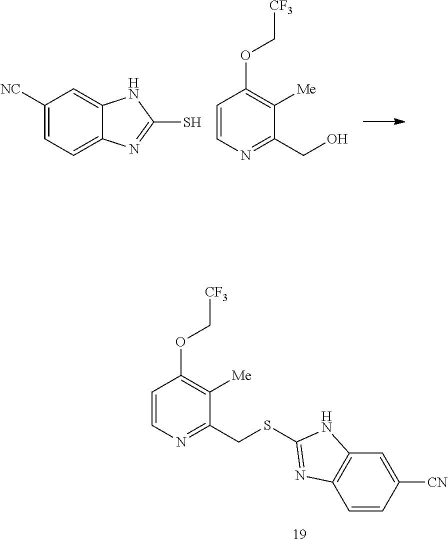

### Preparation of 5-cyano-2-(((3-methyl-4-(2,2,2-trifluoroethoxy)pyridin-2-yl)methyl)thio)-1H-benzo[d]-imidazole (19)

To a conical microwave vial was added 12 (28 mg, 0.13 mmol). Dichloromethane (1 mL) was added, followed by SOCl₂ (14 μL, 0.19 mmol, 1.5 equiv). The mixture turned into a thick white paste. A further 0.7 mL dichloromethane was added and the mixture was heated to 40° C. for 1 h with stirring. The mixture was then transferred to a 4-dram vial and water (0.2 mL) was added. dichloromethane was removed by rotary evaporation. The residue was then taken up in methanol (0.7 mL) and the 5-cyanothiobenzimidazole (22 mg, 0.13 mmol, 1 equiv) was added. The pH was adjusted to 11 with 1N NaOH and the mixture stirred for a further 90 min. The mixture was then poured into a sep. funnel, diluted with 3 mL water and extracted with 10 mL dichloromethane. The aq. phase was washed with a further 10 mL dichloromethane, the organic phases were combined, dried over MgSO₄, filtered and concentrated. Purification by SiO₂ (10 g cartridge, 0 to 100% ethyl acetate in dichloromethane) followed by reverse phase purification (4 g cartridge, 20 to 100% MeCN in H₂O) afforded the final compound as a white solid. 18 mg, 38%. mp 204-205° C. 1H NMR (300 MHz, Chloroform-d) δ 13.32 (s, 1H), 8.36 (d, J=5.8 Hz, 1H), 7.79 (br s, 1H), 7.64-7.39 (br s, 1H), 7.39 (dd, J=8.3, 1.5 Hz, 1H), 6.71 (d, J=5.8 Hz, 1H), 4.41 (q, J=J=7.7 Hz, 2H), 4.35 (s, 2H), 2.27 (s, 3H). 13C NMR (126 MHz, Chloroform-d) δ 162.73, 157.25, 147.35, 125.70, 123.83, 122.01, 121.62, 120.06, 106.24, 65.58 (q, J=36.7 Hz), 34.40, 10.72. ¹⁹F NMR (471 MHz, Chloroform-d) δ −73.72 (t, J=7.8 Hz). HRMS calcd. 379.08349, found 379.08345. IR, 3345.5, 2224.9, 1612.0, 1579.4, 1472.3, 1428.3, 1337.6, 1268.2, 1157.7, 1108.8, 977.4, 915.0, 880.3, 864.8, 817.6, 798.9, 733.4, 663.8.

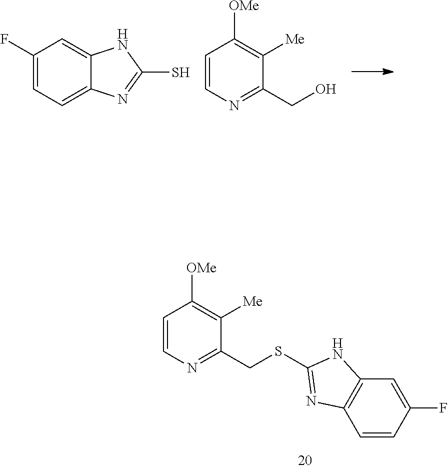

### Preparation of 5-fluoro-2-(((3-methyl-4-methoxypyridin-2-yl)methyl)thio)-1H-benzo[d]imidazole (20)

To a conical microwave vial was added 13 (33 mg, 0.2 mmol, 1.1 equiv). Dichloromethane (0.7 mL was added), followed by SOCl₂ (20 μL, 0.26 mmol, 1.5 equiv). The mixture turned into a thick white paste. A further 0.7 mL dichloromethane was added and the mixture was heated to 40° C. for 1 h with stirring. The mixture was then transferred to a 4-dram vial and water (0.2 mL) was added. Dichloromethane was removed by rotary evaporation. The residue was then treated with methanol (0.7 mL) and the 5-fluorothiobenzimidazole. The pH was adjusted to 11 with 1N NaOH and the mixture stirred for a further 90 min. The mixture was then poured into a sep. funnel, diluted with 3 mL water and extracted with 10 mL dichloromethane. The aq. phase was washed with a further 10 mL dichloromethane, the organic phases were combined, dried over MgSO₄, filtered and concentrated. The product was purified over SiO₂ (10 g cartridge, 10 to 100% ethyl acetate in hexanes) to afford a yellow-brown solid. 33 mg, 62%. mp 153-155° C., 1H NMR (300 MHz, Chloroform-d) δ 13.31 (br s, 1H), 8.40 (d, J=5.7 Hz, 1H), 7.46 (br s, 1H), 7.23 (br s, 1H), 6.95 (ddd, =9.7, 8.7, 2.5 Hz, 1H), 6.81 (d, J=5.8 Hz, 1H), 4.38 (s, 2H), 3.93 (s, 3H), 2.29 (s, 3H). 13C NMR (126 MHz, Chloroform-d) δ 165.03, 160.08, 158.20, 156.39, 147.26, 121.16, 109.75 (d, J=25.4 Hz), 105.48, 55.76, 34.70, 10.67. ¹⁹F NMR (282 MHz, Chloroform-d) δ-121.43. HRMS M+H⁺ calcd. 304.09144, found 304.09125. IR, 2938.5, 1727.7, 1687.2, 1626.8, 1579.4, 1478.6, 1436.4, 1407.8, 1345.3, 1295.4, 1260.5, 1202.2, 1162.2, 1133.2, 1096.0, 1009.6, 954.1, 817.6, 768.1, 733.9.

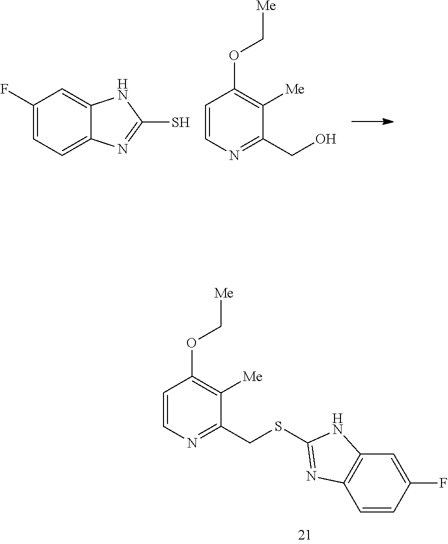

### Preparation of 5-fluoro-2-(((3-methyl-4-ethoxypyridin-2-yl)methyl)thio)-1H-benzo[d]imidazole (21)

To a conical microwave vial was added 14 (25 mg, 0.15 mmol). Dichloromethane (0.7 mL) was added, followed by SOCl₂ (16 μL, 0.22 mol, 1.5 equiv). The mixture turned into a thick white paste. A further 0.7 mL dichloromethane was added and the mixture was heated to 40° C. for 1 h with stirring. The mixture was then transferred to a 4-dram vial and water (0.2 mL) was added. Dichloromethane was removed by rotary evaporation. The residue was then taken up in methanol (0.7 mL) and the 5-fluorothiobenzimidazole (28 mg, 0.17 mmol, 1.1 equiv) was added. The pH was adjusted to 11 with 1N NaOH and the mixture stirred for a further 90 min. The mixture was then poured into a sep. funnel, diluted with 3 mL water and extracted with 10 mL dichloromethane. The aq. phase was washed with a further 10 mL dichloromethane, the organic phases were combined, dried over MgSO₄, filtered and concentrated. Purification by SiO₂ (10 g cartridge, 30% to 50% ethyl acetate in dichloromethane) afforded a yellow solid. 18 mg, 38%: mp 102-104° C., 1H NMR (300 MHz, Chloroform-d) δ 13.36 (br s, 1H), 8.36 (d, J=5.8 Hz, 1H), 7.46 (s, 1H), 7.23 (s, 1H), 6.95 (ddd, J=9.7, 8.7, 2.5 Hz, 1H), 6.78 (d, J=5.8 Hz, 1H), 4.38 (s, 2H), 4.14 (q, J=6.9 Hz, 2H), 2.29 (s, 3H), 1.50 (t, J=7.0 Hz, 3H). 13C NMR (151 MHz, Chloroform-d) δ 164.42, 156.50, 147.11, 121.16, 109.73 (d, J=25.4 Hz), 106.12, 64.25, 34.67, 14.50, 10.74. ¹⁹F NMR (282 MHz, Chloroform-d) δ-121.70. HRMS M+H⁺ calcd. 318.10709, found 318.10665. IR, 2934.3, 1580.2, 1467.1, 1404.3, 1346.3, 1297.0, 1260.4, 1133.0, 1085.2, 1030.2, 953.9, 805.9, 766.9, 728.3.

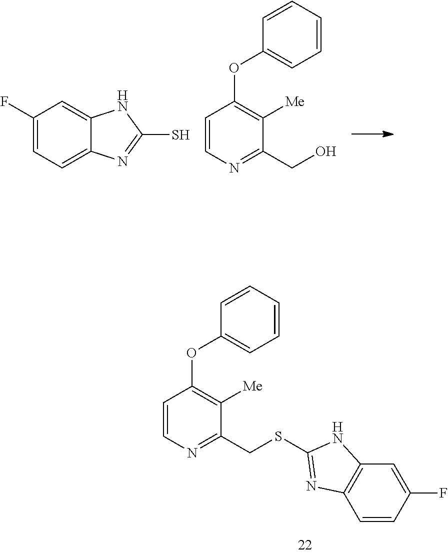

### Preparation of 5-fluoro-2-(((3-methyl-4-phenoxypyridin-2-yl)methyl)thio)-1H-benzo[d]imidazole (22)

To a conical microwave vial was added 15 (46 mg, 0.22 mmol, 1.1 equiv). Dichloromethane (0.7 mL) was added, followed by SOCl₂ (24 μL, 0.29 mmol, 1.5 equiv). The mixture turned into a thick white paste. A further 0.7 mL dichloromethane was added and the mixture was heated to 40° C. for 1 h with stirring. The mixture was then transferred to a 4-dram vial and water (0.2 mL) was added. Dichloromethane was removed by rotary evaporation. The residue was then taken up in methanol (0.7 mL) and the 5-fluorothiobenzimidazole (33 mg, 0.2 mmol) was added. The pH was adjusted to 11 with 1N NaOH and the mixture stirred for a further 90 min. The mixture was then poured into a sep. funnel, diluted with 3 mL water and extracted with 10 mL dichloromethane. The aq. phase was washed with a further 10 mL dichloromethane, the organic phases were combined, dried over MgSO₄, filtered and concentrated. The product was purified over SiO₂ (10 g cartridge, 10% to 100% ethyl acetate in hexanes). Product is streaky so some fractions had to be cut during collection to afford a yellow solid. 51 mg., 71%. mp. 134-136° C. 1H NMR (400 MHz, Chloroform-d) δ 12.88 (s, 1H), 8.20 (d, J=5.7 Hz, 1H), 7.37 (dd, J=8.5, 7.4 Hz, 2H), 7.33 (br s, 1H) 7.22-7.20 (m, 2H), 7.00 (dd, J=8.6, 1.1 Hz, 2H), 6.86 (ddd, J=9.6, 8.7, 2.5 Hz, 1H), 6.50 (d, J=5.7 Hz, 1H), 4.36 (s, 2H), 2.36 (s, 3H). 13C NMR (101 MHz, Chloroform-d) δ 164.41, 160.35, 158.00, 157.76, 154.23, 146.97, 130.34, 125.56, 122.20, 120.67, 109.95 (d, J=5.2 Hz), 109.73, 34.74, 11.03. ¹⁹F NMR (282 MHz, Chloroform-d) δ-121.18. HRMS M+H⁺ calcd. 366.10709, found 366.10620. IR, 2792.7, 1627.5, 1573.8, 1489.3, 1465.4, 1441.3, 1412.7, 1342.4, 1289.0, 1257.1, 1210.4, 1162.3, 1129.0, 1103.7, 1091.9, 1069.4, 966.0, 951.2, 932.1, 907.9, 844.6, 819.3, 796.1, 765.0, 729.8, 710.0, 687, 671.1.

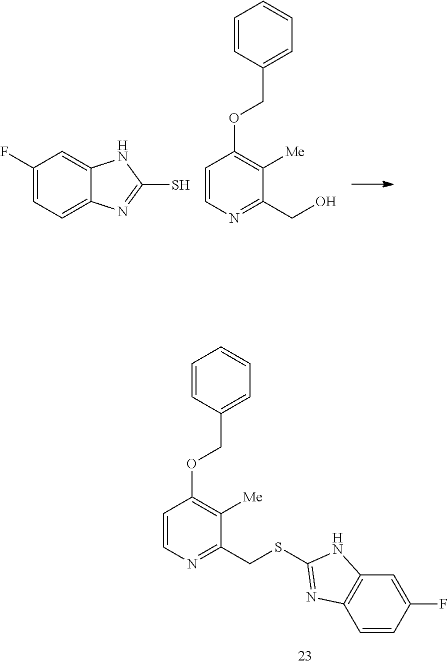

### Preparation of 5-fluoro-2-(((3-methyl-4-benzyloxypyridin-2-yl)methyl)thio)-1H-benzo[d]-imidazole (23)

To a 10 mL microwave vial was added 16 (83 mg, 0.36 mol) and dichloromethane (2 mL). The mixture was sonicated until homogenous. To this vial was then added under stirring SOCl₂ (52 μL, 0.72 mmol, 2 equiv). The vial was capped and stirred at 40° C. for 3 h. The vial was then allowed to cool to room temperature and then placed in an ice bath. MeOH was added dropwise until no further reactivity was observed. The material was then transferred to a 4-dram vial with MeOH washings (4 mL total.) To the vial was then added 4 mL 1N NaOH and the pH was adjusted to >12. The vial was stirred at ambient temperature for 4 h. The mixture was then transferred to a sep. funnel and diluted with 40 mL dichloromethane and 20 mL H₂O. The phases were separated and the aq. phase extracted with a further 20 mL dichloromethane. The organic phases were washed with brine, dried over MgSO₄, filtered and concentrated. Column chromatography on SiO₂ (10 g cartridge, 10% to 100% ethyl acetate in dichloromethane) afforded a yellow solid. 102 mg, 74%. mp 175-177° C. 1H NMR (300 MHz, Chloroform-d) δ 13.17 (s, 1H), 8.36 (d, J=5.7 Hz, 1H), 7.64-7.34 (m, 6H), 7.24 (s, 1H), 6.95 (ddd, J=9.7, 8.7, 2.5 Hz, 1H), 6.85 (d, J=5.8 Hz, 1H), 5.18 (s, 2H), 4.40 (s, 2H), 2.34 (s, 3H). 13C NMR (76 MHz, Chloroform-d) δ 164.10, 160.72, 157.58, 156.71, 147.19, 135.50, 128.81, 128.45, 127.21, 121.45, 109.76 (d, J=25.5 Hz), 106.67, 70.34, 34.74, 10.92. ¹⁹F NMR (282 MHz, Chloroform-d) δ-121.31 (br s). HRMS M+H⁺ calcd. 380.12274, found 380.12125. IR, 3070.3, 2882.8, 1579.1, 1499.0, 1478.6, 1446.6, 1418.4, 1345.0, 1299.0, 1280.6, 1256, 1201.6, 1128.8, 1087.8, 989.0, 971.5, 950.8, 916.3, 899.8, 864.7, 842.6, 820.8, 793.7, 729.4, 693.0, 661.5.

### 2.3.2 Oxidations

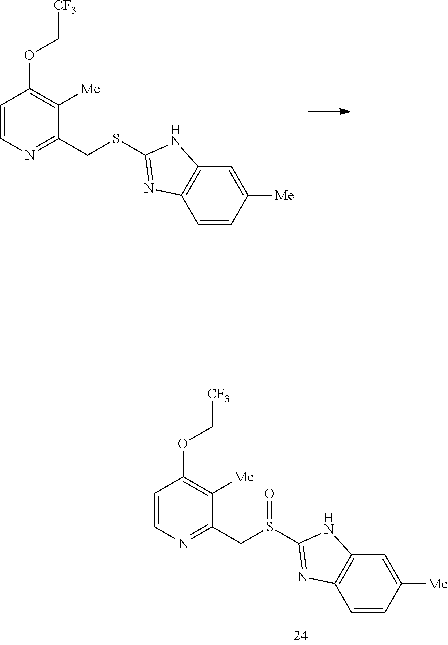 [SMILES: Cc1ccc2nc(SCc3nccc(OCC(F)(F)F)c3C)[nH]c2c1 ; Cc1ccc2nc(S(=O)Cc3nccc(OCC(F)(F)F)c3C)[nH]c2c1]

### Preparation of 5-methyl-2-(((3-methyl-4-(2,2,2-trifluoroethoxy)pyridin-2-yl)methyl)sulfinyl)-1H-benzo[d]-imidazole (24)

To a 4-dram vial was added 18 (55 mg, 0.15 mmol), MeOH (1 mL), and VO(acac)₂ (4 mg, 0.1 equiv). The mixture was sonicated until the vanadium formed a fine dispersion. H₂O₂ (35%, 5 drops) was added via pipette and the reaction allowed to stir for 45 min. For purification, the mixtures were placed in a sep. funnel and diluted with 20 mL dichloromethane. 5 mL sat. aq. thiosulfate was added and the phases separated. The organic phase was washed with brine (10 mL), dried over MgSO₄, filtered and concentrated. Purification by SiO₂ (10 g cartridge, 50% to 100% ethyl acetate in dichloromethane) afforded 37 mg of the desired product slightly impure. A second column (10 g cartridge, 10% to 100% ethyl acetate in dichloromethane) was performed to afford a white solid. 33 mg, 58%. mp. >133C (dec.) 1H NMR (300 MHz, Chloroform-d) δ 11.48 (br s, 1H), 8.37 (d, J=5.6 Hz, 1H), 7.78-7.50 (br s, 1H), 7.43 (br s, 1H), 7.17 (dd, J=8.3, 1.5 Hz, 1H), 6.69 (d, J=5.6 Hz, 1H), 4.84 (d, J=13.7 Hz, 1H), 4.72 (d, J=13.7 Hz, 1H), 4.38 (q, J=7.8 Hz, 2H), 2.50 (s, 3H), 2.23 (s, 3H). 13C NMR (76 MHz, DMSO-d₆) δ 161.75, 151.39, 148.58, 122.51, 107.48, 65.11 (q, J=34.7 Hz), 60.43, 21.76, 11.03. ¹⁹F NMR (282 MHz, Chloroform-d) δ-73.80 (t, J=7.8 Hz). HRMS M+H⁺ calcd. 384.09881 found. 384.09778. IR, 3016.5, 2236.1, 1586.1, 1479.8, 1451.5, 1328.3, 1315.9, 1282.7, 1258.4, 1235.0, 1201.0, 1154.4, 1131.6, 1109.0, 1085.0, 1037.9, 969.1, 912.4, 883.1, 857.9, 838.7, 809.9, 753.4, 727.2, 657.9.

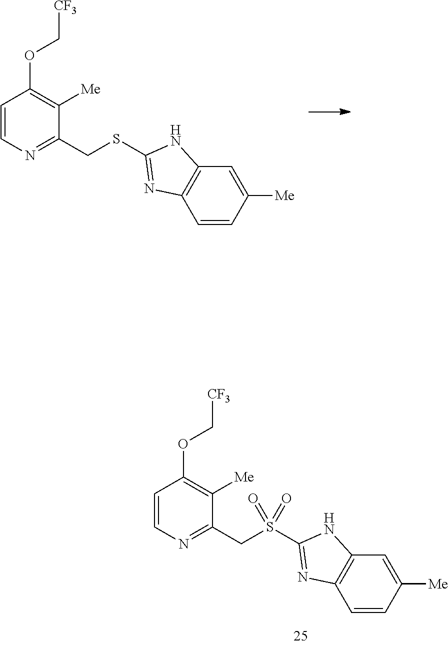 [SMILES: Cc1ccc2nc(SCc3nccc(OCC(F)(F)F)c3C)[nH]c2c1 ; Cc1ccc2nc(S(=O)(=O)Cc3nccc(OCC(F)(F)F)c3C)[nH]c2c1]

### Preparation of 5-methyl-2-(((3-methyl-4-(2,2,2-trifluoroethoxy)pyridin-2-yl)methyl)sulfonyl)-1H-benzo[d]-imidazole (25)

To a 1 dram vial was added 18 (100 mg, 0.27 mmol), dichloromethane (2 mL). mCPBA (77%, 92 mg, 0.40 mmol, 1.5 equiv) was added directly and the vial was allowed to stir for 1 h. Immediately upon addition, colorization of the reaction occurred and the mixture continued to darken until red-black. After 1 h, the mixture was directly placed onto a column and purified by chromatography (10 g cartridge, 0 to 100% ethyl acetate in dichloromethane). Obtained 43 mg of sulfone contaminated with CPBA (potentially CPBA salt). Extraction with Na₂S₂O₃ and sat aq. carbonate (5 mL: 10 mL) and dichloromethane 25 mL cleanly afforded a white solid. 31 mg, 29% mp. 193-194° C. 1H NMR (500 MHz, DMSO-d₆) δ 13.62 (s, 1H), 8.12 (d, J=5.6 Hz, 1H), 7.52 (d, J=86.7 Hz, 2H), 7.21 (d, J=8.4 Hz, 1H), 7.08 (d, J=5.7 Hz, 1H), 5.09 (s, 2H), 4.91 (q, J=8.7 Hz, 2H), 2.46 (s, 3H), 2.22 (s, 3H). 13C NMR (76 MHz, DMSO-d₆) δ 161.91, 148.53, 148.39, 123.73, 107.89, 65.14 (q, J=34.3 Hz), 60.93, 21.82, 11.39. HRMS M+H⁺ calcd. 400.09372 found 400.09261, IR 3250.9, 2987.7, 1578.6, 1474.7, 1452.6, 1402.0, 1265.7, 1250.4, 1159.2, 1115.8, 1038.2, 978.7, 920.6, 859.5, 828.0, 803.7, 771.0, 735.3, 687.9, 675.0, 656.2.

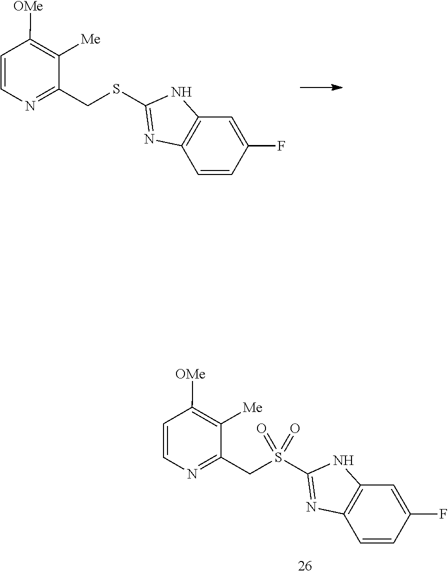 [SMILES: COc1ccnc(CSc2nc3ccc(F)cc3[nH]2)c1C ; COc1ccnc(CS(=O)(=O)c2nc3ccc(F)cc3[nH]2)c1C]

### Preparation of 5-fluoro-2-(((3-methyl-4-methoxypyridin-2-yl)methyl)sulfonyl)-1H-benzo[d]-imidazole (26)

To a 4-dram vial was added 20 (30 mg, 0.1 mmol) and dichloromethane (3 mL). The mixture was stirred until homogenous. mCPBA (0.1M in dichloromethane, 2 mL, 0.2 mmol, 2 equiv) was then added dropwise. The reaction was stirred for a further 2 h and then quenched with Na₂S₂O₃ (1 mL). The mixture was poured into a sep. funnel and diluted with 5 mL H₂O. The aq. phase was extracted with EtOAc (2×20 mL), the organic phases were combined and washed with brine (10 mL). The organic phase was dried over MgSO₄, filtered and concentrated. SiO₂ purification (10 g cartridge, 10 to 100% ethyl acetate in dichloromethane) afforded a yellow solid. 21 mg, 63%. mp. 192-193° C. 1H NMR (500 MHz, DMSO-d₆) δ 13.90 (s, 1H), 8.04 (d, J=5.6 Hz, 1H), 7.73 (dd, J=9.0, 4.8 Hz, 1H), 7.49 (d, J=10.0 Hz, 1H), 7.28 (td, J=9.4, 2.5 Hz, 1H), 6.96 (d, J=5.6 Hz, 1H), 5.08 (s, 2H), 3.86 (s, 3H), 2.19 (s, 3H). 13C NMR (126 MHz, DMSO-d₆) δ 164.24, 161.09, 159.20, 149.44, 148.58, 147.45, 123.52, 113.86 (d, J=26.2 Hz), 106.80, 60.92, 56.33, 11.57. HRMS M+H⁺ calcd. 334.06562, found 334.06584. IR, 2929.3, 1587.8, 1479.0, 1435.6, 1333.0, 1302.6, 1232.7, 1207.3, 1129.1, 1100.2, 1081.5, 1006.1, 965.5, 924.9, 875.5, 837.2, 815.2, 732.6.

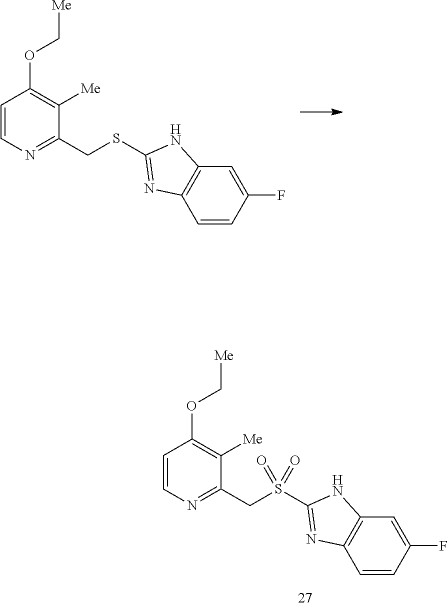 [SMILES: CCOc1ccnc(CSc2nc3ccc(F)cc3[nH]2)c1C ; CCOc1ccnc(CS(=O)(=O)c2nc3ccc(F)cc3[nH]2)c1C]

### Preparation of 5-fluoro-2-(((3-methyl-4-ethoxypyridin-2-yl)methyl)sulfonyl)-1H-benzo[d]-imidazole (27)

To a 4-dram vial was added 21 (31.7 mg, 0.1 mmol), sodium phosphate dibasic (70 mg, 0.5 mmol, 5 equiv), and dichloromethane 2 mL. The mixture was stirred until homogenous. mCPBA (0.1 M solution in dichloromethane, 2 mL, 0.2 mmol, 2 equiv) was added dropwise. The reaction was stirred for a further 2 h and then quenched with Na₂S₂O₃ (1 mL). The mixture was diluted with water (5 mL) and extracted twice with dichloromethane (2×20 mL). The organic phases were combined and washed with brine (10 mL). SiO₂ purification (10 g cartridge, 10% to 100% ethyl acetate in dichloromethane) afforded the product as a yellow solid. 8.5 mg 25%, mp 177-178° C. 1H NMR (300 MHz, DMSO-d₆) δ 8.28 (s, 1H), 8.01 (d, J=5.6 Hz, 1H), 7.71 (dd, J=9.0, 4.8 Hz, 1H), 7.44 (dd, J=9.2, 2.5 Hz, 1H), 7.24 (td, J=9.4, 2.5 Hz, 1H), 6.90 (d, J=5.6 Hz, 1H), 5.04 (s, 2H), 4.11 (q, J=7.1 Hz, 2H), 2.19 (s, 3H), 1.37 (t, J=7.0 Hz, 3H). 13C NMR (151 MHz, DMSO-d₆) δ 163.45, 148.43, 147.46, 123.55, 107.24, 64.29, 60.95, 55.25, 14.81, 11.57. HRMS M+H⁺ calcd. 350.09692 found 350.09667. IR, 2938.3, 1628.6, 1589.1, 1483.6, 1465.9, 1333.8, 1308.2, 1207.8, 1130.6, 1081.9, 1026.9, 963.4, 831.6, 804.2, 731.0.

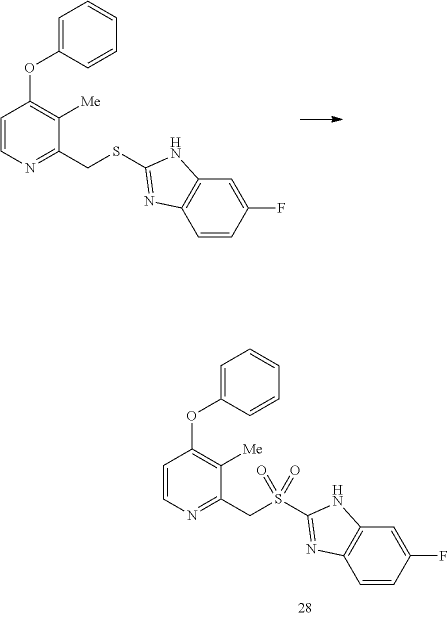 [SMILES: Cc1c(Oc2ccccc2)ccnc1CSc1nc2ccc(F)cc2[nH]1 ; Cc1c(Oc2ccccc2)ccnc1CS(=O)(=O)c1nc2ccc(F)cc2[nH]1]

### Preparation of 5-fluoro-2-(((3-methyl-4-phenoxypyridin-2-yl)methyl)sulfonyl)-1H-benzo[d]-imidazole (28)

To a 4-dram vial was added 22 (37 mg, 0.1 mmol), 2 mL dichloromethane and sodium phosphate dibasic (70 mg, 0.5 mmol, 5 equiv) and the mixture was cooled to 0° C. 115 mg of mCPBA (77%) was dissolved in 1 mL dichloromethane and 200 μL of this solution was added dropwise at 0° C. (23 mg, 0.1 mmol, 1 equiv). The mixture was stirred 1 h at 0° C. and warmed to ambient temperature over 1 h. The mixture was quenched with sat. aq. thiosulfate (1 mL), diluted with water (10 mL), and extracted with dichloromethane (2×20 mL). The organic phases were combined, washed with brine (10 mL) and dried over MgSO₄, filtered and concentrated. Purification by SiO₂ (10 g cartridge, 0 to 75% ethyl acetate in dichloromethane) afforded the product as a brown solid. 18 mg, 45% mp. 155-157° C. 1H NMR (300 MHz, Chloroform-d) δ 8.15 (d, J=5.7 Hz, 1H), 7.61 (s, 1H), 7.53-7.38 (m, 2H), 7.37-7.25 (m, 2H), 7.23 (s, 3H), 7.16-7.00 (m, 3H), 6.57 (d, J=5.7 Hz, 1H), 5.15 (s, 2H), 2.53 (s, 3H). 13C NMR (76 MHz, Chloroform-d) δ 164.48, 154.01, 148.60, 147.86, 147.61, 130.36, 125.70 (d, J=1.9 Hz), 120.72, 110.33, 60.61, 11.80. ¹⁹F NMR (282 MHz, Chloroform-d) δ-113.54. HRMS, M+H⁺ calcd. 398.09692 found 398.09731, IR, 2988.8, 1629.0, 1577.2, 1511.2, 1488.4, 1469.3, 1328.8, 1279.5, 1239.3, 1204.9, 1127.5, 1069.0, 971.9, 919.3, 878.6, 855.7, 830.7, 807.1, 723.3, 689.9.

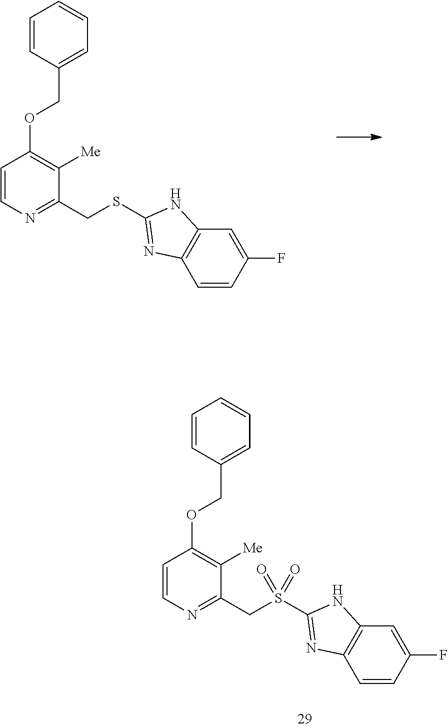 [SMILES: Cc1c(OCc2ccccc2)ccnc1CSc1nc2ccc(F)cc2[nH]1 ; Cc1c(OCc2ccccc2)ccnc1CS(=O)(=O)c1nc2ccc(F)cc2[nH]1]

### Preparation of 5-fluoro-2-(((3-methyl-4-benzyloxypyridin-2-yl)methyl)sulfonyl)-1H-benzo[d]-imidazole (29)

To a 4-dram vial was added 23 (38 mg, 0.1 mmol), sodium phosphate dibasic (70 mg, 0.5 mmol, 5 equiv) and dichloromethane 2 mL. The mixture was stirred until homogenous. mCPBA (77%, 0.1 M in dichloromethane 2 mL, 0.2 mmol 2 equiv) was then added dropwise. The reaction was stirred for a further 2 h and then quenched with aq. Na₂S₂O₃ (1 mL). The mixture was diluted with water and extracted with EtOAc (2×20 mL). The organic phases were combined, washed with brine (10 mL), dried over MgSO₄, filtered and concentrated. Purification by SiO₂ (10 g cartridge, 10 to 100% ethyl acetate in dichloromethane) afforded a yellow solid. 26 mg, 63%. mp. 185-188° C. 1H NMR (300 MHz, DMSO-d₆) δ 13.80 (s, 1H), 8.06 (d, J=5.6 Hz, 1H), 7.94-7.83 (m, 1H), 7.70 (d, J=7.3 Hz, 1H), 7.61-7.14 (m, 7H), 7.04 (d, J=5.7 Hz, 1H), 5.22 (s, 2H), 5.08 (s, 2H), 2.23 (s, 3H). 13C NMR (76 MHz, DMSO-d₆) δ 163.22, 148.46, 147.83, 136.71, 131.06, 129.28, 129.04, 128.53, 128.36, 127.96, 123.82, 107.84, 70.03, 60.90, 11.73. HRMS M+H⁺ calcd. 410.09692 found 410.09665. IR 3013.1, 1582.1, 1477.0, 1451.2, 1328.6, 1298.6, 1200.8, 1128.9, 1074.5, 971.3, 902.8, 833.2, 737.0, 721.9, 697.8.

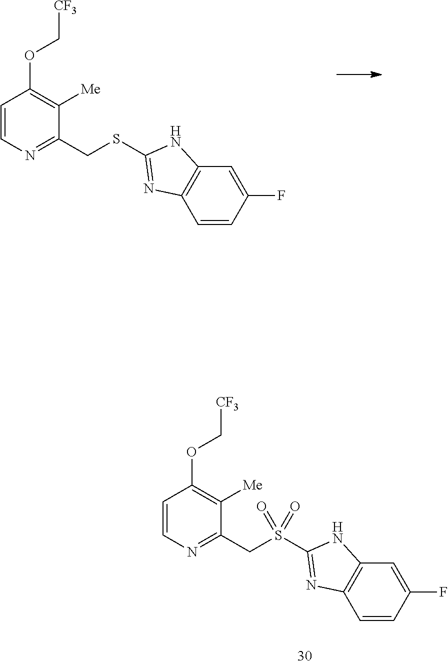 [SMILES: Cc1c(OCC(F)(F)F)ccnc1CSc1nc2ccc(F)cc2[nH]1 ; Cc1c(OCC(F)(F)F)ccnc1CS(=O)(=O)c1nc2ccc(F)cc2[nH]1]

### Preparation of 5-fluoro-2-(((3-methyl-4-(2,2,2)-trifluoroethoxypyridin-2-yl)methyl)sulfonyl)-1H-benzo[d]-imidazole (30)

To a 4-dram vial was added 17 (63 mg, 0.17 mmol) followed by 1 mL MeOH. VO(acac)₂ (3 mg, 0.011 mmol, 0.07 equiv) was added as a solid and the mixture sonicated until homogenous. Under stirring, 0.1 mL of 35% H₂O₂ was added dropwise. The mixture was allowed to stir for 1 h. Then, a further 3 mg of vanadium (0.07 mmol) and 0.1 mL of 35% H₂O₂ was added and the reaction stirred a further 2 h. The reaction was diluted with dichloromethane (10 mL) and transferred to a sep. funnel. The reaction was then washed with aq. Na₂S₂O₃ (5 mL) and the aq. phase was then extracted with a further 10 mL of dichloromethane. The combined organic phases were washed with brine (10 mL) dried over MgSO₄ filtered and concentrated. Purification by SiO₂ (10 g cartridge, 0 to 100% ethyl acetate in dichloromethane) afforded 2 compounds: 16 mg of sulfone at about 50% EtOAc and 12 mg of impure sulfoxide at 90% EtOAc, the latter of which decomposed over time. White solid, 16 mg, 25%. mp 232-233° C. 1H NMR (300 MHz, DMSO-d₆) δ 8.11 (d, J=5.6 Hz, 1H), 7.72 (dd, J=9.0, 4.9 Hz, 1H), 7.52-7.42 (m, 1H), 7.26 (td, J=9.4, 2.5 Hz, 1H), 7.08 (d, J=5.7 Hz, 1H), 5.11 (s, 2H), 4.91 (q, J=8.7 Hz, 2H), 2.24 (s, 3H). 13C NMR (151 MHz, DMSO-d₆) δ 161.91, 148.54, 148.39, 124.6 (q, J=277.3 Hz), 123.74, 123.30, 107.92, 65.11 (q, J=34.5 Hz), 60.80, 55.38, 11.40. ¹⁹F NMR (282 MHz, Chloroform-d) δ-67.95 (t, J=8.7 Hz). HRMS M+H⁺ calcd. 404.06865 found 404.06741. IR 3022.0, 1626.1, 1585.0, 1509.4, 1478.3, 1449.9, 1326.3, 1282.4, 1257.7, 1198.7, 1174.3, 1155.1, 1132.0, 1109.9, 1084.0, 1039.6, 966.4, 918.3, 884.5, 857.3, 839.0, 825.0, 795.1, 732.7, 656.8.

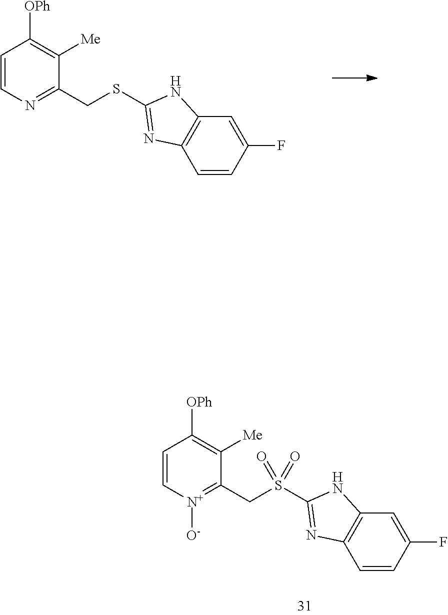 [SMILES: Cc1c(Oc2ccccc2)ccnc1CSc1nc2ccc(F)cc2[nH]1 ; Cc1c(Oc2ccccc2)cc[n+]([O-])c1CS(=O)(=O)c1nc2ccc(F)cc2[nH]1]

### Preparation of 5-fluoro-2-(((1-oxo-3-methyl-4-phenoxypyridin-2-yl)methyl)sulfonyl)-1H-benzo[d]-imidazole (31)

To a 4-dram vial was added 22 (37 mg, 0.1 mmol), dichloromethane (2 mL). NaHCO₃ (25 mg, 0.3 mmol, 3 equiv) was added as a solid and the mixture sonicated until a fine dispersion was achieved. To this was added mCPBA (77%, 57 mg, 2.5 equiv) as a solid with stirring and the reaction was allowed to proceed for 4 h. Observed formation of 2 products (sulfone, n-oxide) by LCMS. The mixture was quenched with aq. thiosulfate (2 mL), diluted with water (5 mL) and then extracted with dichloromethane (20 mL twice). The organic phases were combined, washed with brine (10 mL) dried over MgSO₄, filtered and concentrated. Purification by SiO₂ (10 g cartridge, 0 to 5% MeOH in dichloromethane) afforded the n-oxide as a white powder. 22 mg, 51% mp. 233-235° C. 1H NMR (300 MHz, Chloroform-d) δ 8.12 (d, J=7.2 Hz, 1H), 7.63-7.41 (m, 2H), 7.40-7.24 (m, 2H), 7.19-7.06 (m, 2H), 7.01 (td, J=9.2, 2.4 Hz, 2H), 6.69 (d, J=7.2 Hz, 1H), 5.45 (s, 2H), 2.63 (s, 3H). 13C NMR (76 MHz, Chloroform-d) δ 156.71, 153.83, 149.47, 141.10, 137.82, 130.63, 128.95, 126.08, 120.40, 112.09, 55.49, 13.14. HRMS M+H⁺ calcd. 414.09183 found 414.09190. IR, 2924.1, 2521.6, 1625.9, 1588.7, 1507.0, 1489.6, 1436.4, 1341.4, 1327.5, 1282.3, 1251.6, 1197.0, 1140.6, 1130.1, 1105.6, 1073.5, 1016.9, 963.0, 921.1, 828.8, 785.4, 732.8, 707.5, 690.4.

### 2.3.3. Oxoquinazoline Series

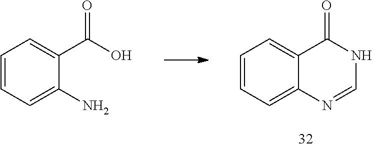 [SMILES: Nc1ccccc1C(=O)O ; O=c1[nH]cnc2ccccc12]

### Preparation of 2-oxoquinazoline (32)

To a microwave vial was added anthranilic acid (1 g, 7.3 mmol) and formamide (1.65 g, 1.5 mL, 5 equiv). The vial was crimped and then heated in a microwave with stirring for 30 min at 150° C. The vial was allowed to cool down to room temperature, uncrimped, and transferred into a 25 mL RB-flask with ethyl acetate washes. The volatiles were removed by rotary evaporation and the product was crystallized out of EtOH (4 mL) to afford 886 mg of pdt which was contaminated with formamide. The solid was washed with water (15 mL) and dried under airflow to obtain a white solid. 773 mg, 73% 1H NMR (400 MHz, Chloroform-d) δ 11.00 (s, 1H), 8.37-8.31 (m, 1H), 8.26 (d, J=13.6 Hz, 1H), 8.12 (s, 1H), 7.93-7.74 (m, 2H), 7.57 (ddd, J=8.2, 6.9, 1.5 Hz, 1H). Spectral data are in accordance with literature. Abdullaha et al., 2019.

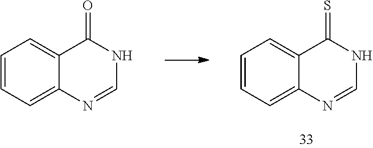 [SMILES: O=c1[nH]cnc2ccccc12 ; S=c1[nH]cnc2ccccc12]

### Preparation of 2-thioquinazoline (33)

To a 30 mL microwave vial was added 32 (220 mg, 1.5 mmol), pyridine (3 mL), followed by Lawesson's reagent (487 mg, 1.2 mmol, 0.8 equiv). The vial was capped and heated in a microwave reactor at 120° C. for 20 mins. The mixture was transferred to a RB-flask and the volatiles were evaporated. The residue was taken up in 8 ml of boiling water (not soluble) and filtered. The cake was washed with a further 5 mL of water, then the cake was collected by dissolving in 1N NaOH. To the aqueous solution was then added sat. aq. NH₄C₁ until all product precipitated. The product was then filtered to remove the water, washed with water (5-10 mL) and the cake was then collected by dissolving in copious EtOAc (MeOH does not work) to afford a white solid. 197 mg, 81% 1H NMR (500 MHz, DMSO-d₆) δ 13.88 (s, 1H), 8.58 (dd, J=8.2, 1.6 Hz, 1H), 8.18 (s, 1H), 7.91 (ddd, J=8.4, 7.0, 1.5 Hz, 1H), 7.74 (dd, J=8.2, 1.1 Hz, 1H), 7.63 (ddd, J=8.3, 7.0, 1.3 Hz, 1H). Spectral data are in accordance with the literature. Alexandre et al., 2003.

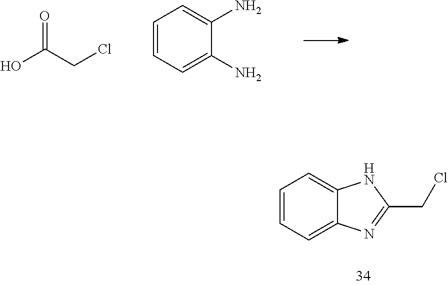 [SMILES: O=C(O)CCl ; Nc1ccccc1N ; ClCc1nc2ccccc2[nH]1]

### Preparation of 2-chloromethylbenzimidazole (34)

To a 100 mL RB flask was added chloroacetic acid (4.7 g, 50 mmol), followed by 25 mL 4N HCl. To this was added phenylene diamine (5.4 g, 50 mmol, 1 equiv) and the mixture was heated to 100° C. for 4 h. The residue was basified with sodium carbonate to pH >7 and the resulting solid was filtered, washed with water, and then collected by dissolving in EtOAc and concentrated to afford a slightly green powder. 7.21 g, 87%: 1H NMR (300 MHz, Chloroform-d) δ 7.63 (br s, 2H), 7.32 (dd, J=6.1, 3.2 Hz, 2H), 4.89 (s, 2H). Spectral data are in agreement with the literature. Dayakar et al., 2015.

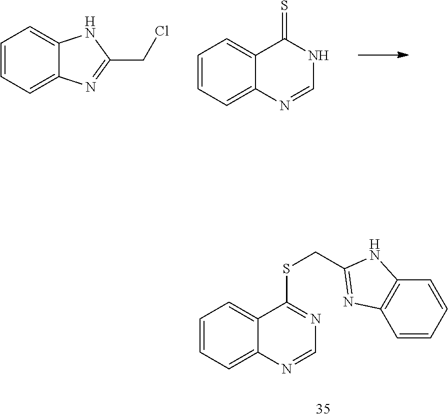

### Preparation of 4-(((1H-benzo[d]imidazol-2-yl)methyl)thio)quinazoline (35)

To a RB-flask containing 33 (215 mg, 1.3 mmol) was added MeOH (10 mL), chloromethylbenzimidazole (332 mg, 2 mmol, 1.5 equiv) and sodium methoxide (358 mg, 6.6 mmol, 5 equiv). The mixture was then stirred for 3 h. The pH was adjusted to 7 with sat. aq. NaHCO₃ and then extracted with dichloromethane (3×20 mL). The organic phases were combined, washed with brine (10 mL) dried over MgSO₄, filtered and concentrated. The product was purified by SiO₂ (10 g cartridge, 10% to 100% ethyl acetate in hexanes) to afford 320 mg of an impure white solid. Recrystallization from acetone (˜2 mL) afforded the desired product as a white solid. 111 mg, 29%: mp 198-200 (dec.) 1H NMR (500 MHz, Chloroform-d) δ 10.61 (s, 1H), 9.05 (s, 1H), 7.94 (dd, J=8.4, 1.3 Hz, 1H), 7.91 (dt, J=8.5, 0.8 Hz, 1H), 7.81 (ddd, J=8.5, 6.9, 1.4 Hz, 1H), 7.51 (ddd, J=8.3, 6.9, 1.2 Hz, 1H), 7.18-7.12 (m, 2H), 4.75 (s, 2H). 13C NMR (126 MHz, Chloroform-d) δ 171.10, 153.06, 151.24, 148.04, 134.55, 128.88, 128.03, 123.88, 123.58, 122.80, 27.22. HRMS M+H⁺ calcd. 293.08554 found 293.08502, IR 3050.8, 1612.4, 1561.5, 1544.3, 1523.7, 1483.7, 1452.7, 1417.9, 1392.3, 1319.2, 1268.8, 1229.3, 1203.2, 1144.1, 1107.7, 1008.8, 996.2, 965.3, 903.5, 876.2, 844.3, 761.7, 741.3, 698.5, 676.

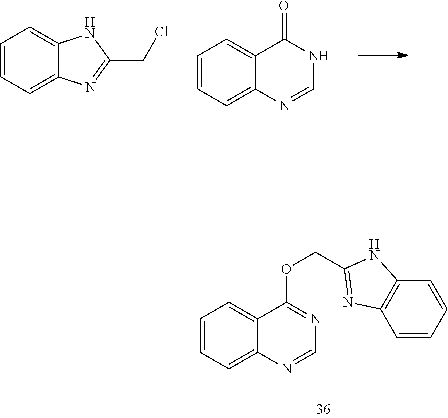

### Preparation of 4-(((1H-benzo[d]imidazol-2-yl)methoxy)quinazoline (36)

To a RB-flask containing the 32 (291 mg, 2 mmol) was added MeOH (10 mL), chloromethylbenzimidazole (498 mg, 3 mmol, 1.5 equiv) and sodium methoxide (540 mg, 10 mmol, 5 equiv). The mixture was then stirred for 3 h. The pH was adjusted to 7 with sat. aq. NaHCO₃ and then extracted with dichloromethane (3×20 mL). The organic phases were combined, washed with brine (10 mL) dried over MgSO₄, filtered and concentrated. The product was purified by SiO₂ (10 g cartridge, 10% to 100% ethyl acetate in hexanes) to afford a complex mixture. The mixture was then repurified using SiO₂ (10 g cartridge, 0 to 100% ethyl acetate in dichloromethane). Three peaks were identified. The first was methanolyzed, while the second was the oxoquinazoline 36. The third peak was a mixture and was repurified by SiO₂ (10 g cartridge, 0 to 10% MeOH in dichloromethane). The peak corresponding to impure product was then subjected to chromatography on reverse-phase (4 g cartridge, 10 to 100% MeCN in H₂O) to afford 7 mg of a white solid. mp >270° C. (dec.) 1H NMR (500 MHz, Chloroform-d) δ 10.37 (s, 1H), 8.34-8.19 (m, 2H), 7.73 (ddd, J=8.4, 7.0, 1.5 Hz, 1H), 7.67 (dd, J=8.2, 1.2 Hz, 1H), 7.58-7.47 (m, 3H), 7.21 (q, J=3.5, 3.0 Hz, 2H), 5.37 (s, 2H). 13C NMR (126 MHz, Chloroform-d) δ 162.59, 148.32, 148.16, 145.72, 134.99, 127.95, 127.83, 126.50, 123.37, 121.65, 45.51, 29.71. HRMS M+H⁺ calcd. 277.10839 found 277.10925 IR, 3352.9, 1611.1, 1544.1, 1429.2, 1361.4, 1025.7.

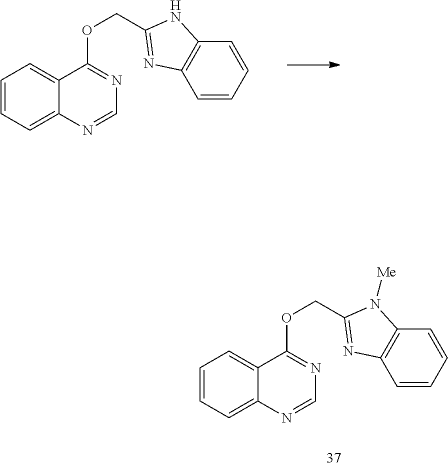 [SMILES: c1ccc2[nH]c(COc3ncnc4ccccc34)nc2c1 ; Cn1c(COc2ncnc3ccccc23)nc2ccccc21]

### 4-((1-methyl-1H-benzo[d]imidazol-2-yl)methoxy)quinazoline (37)

To a dried microwave vial was added 36 (45 mg, 0.15 mmol) in DMF (1 mL). K₂CO₃ (43 mg, 0.3 mmol, 2 equiv) was added as a solid, followed by MeI (10 μL, 0.15 mmol, equiv). The reaction was allowed to stir overnight at 50° C. The mixture was then extracted with 10 mL each of dichloromethane/water, dried over MgSO₄, filtered and concentrated. Purification by SiO₂ (10 g cartridge, 0% to 100% ethyl acetate in dichloromethane) afforded product as a white solid. 34 mg, 71%. 1H NMR (300 MHz, Chloroform-d) δ 8.50 (s, 1H), 8.32 (d, J=8.0 Hz, 1H), 7.77 (ddt, J=6.6, 5.0, 2.3 Hz, 3H), 7.61-7.48 (m, 1H), 7.41-7.29 (m, 3H), 5.53 (s, 2H), 3.99 (s, 3H). 13C NMR (126 MHz, Chloroform-d) δ 160.78, 148.57, 148.04, 146.11, 142.34, 135.84, 134.61, 127.79, 127.51, 126.74, 123.42, 122.67, 121.76, 119.88, 109.77, 40.88, 30.39. HRMS M+H⁺ calcd. 291.12404 found 291.12395. IR, 1681.3, 1612.0, 1473.8, 1275.7, 1260.8, 749.9.

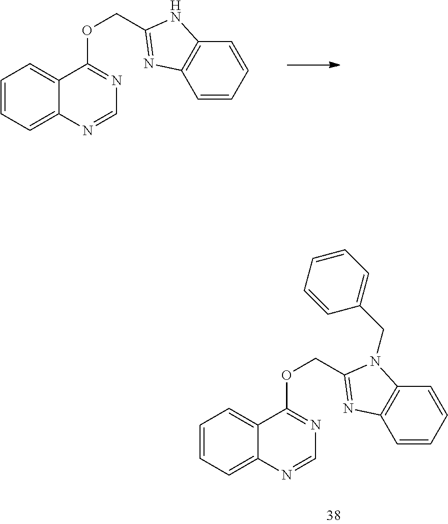 [SMILES: c1ccc2[nH]c(COc3ncnc4ccccc34)nc2c1 ; c1ccc(Cn2c(COc3ncnc4ccccc34)nc3ccccc32)cc1]

### 4-((1-benzyl-1H-benzo[d]imidazol-2-yl)methoxy)quinazoline (38)

To a dried microwave vial was added 36 (45 mg, 0.15 mmol) in DMF (1 mL). K₂CO₃ (43 mg, 0.3 mmol, 2 equiv) was added as a solid, followed by BnCl (17 μL 0.15 mmol, 1 equiv). The reaction was allowed to stir for 18 h at 50° C. The mixture was then extracted with 10 mL each of dichloromethane/water, dried over MgSO₄, filtered and concentrated. Purification by SiO₂ (10 g cartridge, 0% to 70% ethyl acetate in dichloromethane) to afford product as a white solid. 53 mg, 89%. 1H NMR (500 MHz, Chloroform-d) δ 8.35 (s, 1H), 8.18 (dd, J=8.0, 1.5 Hz, 1H), 7.86-7.77 (m, 1H), 7.72 (ddd, J=8.5, 7.1, 1.6 Hz, 1H), 7.64 (dd, J=8.2, 1.2 Hz, 1H), 7.46 (ddd, J=8.1, 7.0, 1.2 Hz, 1H), 7.37-7.27 (m, 3H), 7.11 (dd, J=8.3, 6.9 Hz, 2H), 7.05-7.00 (m, 1H), 6.97-6.87 (m, 2H), 5.64 (s, 2H), 5.47 (s, 2H). 13C NMR (126 MHz, Chloroform-d) δ 160.55, 148.94, 147.79, 145.74, 142.38, 135.73 (d, J=14.9 Hz), 134.37, 128.78, 127.61 (d, J=2.6 Hz), 127.30, 126.71, 125.60, 123.80, 122.86, 121.60, 120.13, 110.13, 47.05, 40.90. HRMS M+H⁺ calcd. 367.15534 found 367.15523. IR 3050.9, 2924.6, 1674.3, 1607.7, 1561.4, 1497.1, 1464.1, 1437.3, 1400.2, 1364.8, 1321.1, 1285.8, 1247.1, 1230.7, 1171.4, 1077.1, 1026.9, 1012.1, 982.9, 954.2, 938.0, 916.0, 885.6, 872.4, 789.0, 772.9, 741.0, 720.6, 697.7.

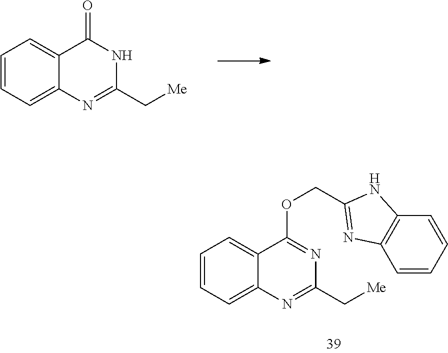 [SMILES: CCc1nc2ccccc2c(=O)[nH]1 ; CCc1nc(OCc2nc3ccccc3[nH]2)c2ccccc2n1]

### Preparation of 2-ethyl-4-(((1H-benzo[d]imidazol-2-yl)methoxy)quinazoline (39)

To a 50-mL RB-flask was added substrate (0.15 g, 0.86 mmol), followed by 10 mL of DMF. Under rapid stirring, NaH (60% in oil, 51 mg, 1.3 mmol, 1.5 equiv) was added in one batch. Then, chloromethylbenzimidazole (215 mg, 1.3 mmol, 1.5 equiv) was added. The reaction was then heated to 80° C. for 3 h. The mixture was allowed to cool to rt and poured into a sep. funnel, acidified with sat. aq. NH₄Cl (3 mL), and then extracted with 30 mL each of ethyl acetate and water. The aq. phase was washed with a further 30 mL of ethyl acetate, the organic phases were combined and washed with brine (15 mL), dried over MgSO₄, filtered and concentrated. Purification by column chromatography (10 g SiO₂, 10-100% EtOAc in dichloromethane, then 10 g SiO₂ 0-4% MeOH in dichloromethane) afforded the desired product as a white solid. 66 mg, 25%. ¹H NMR (500 MHz, DMSO-d₆) δ 12.51 (s, 1H), 8.13 (dd, J=7.9, 1.5 Hz, 1H), 7.85 (ddd, J=8.5, 7.1, 1.6 Hz, 1H), 7.69 (dd, J=8.3, 1.1 Hz, 1H), 7.53 (ddd, J=8.2, 7.1, 1.2 Hz, 1H), 7.46 (br s, 2H), 7.15 (br s, 2H), 5.56 (s, 2H), 2.97 (q, J=7.2 Hz, 2H), 1.27 (t, J=7.2 Hz, 3H). 13C NMR (126 MHz, DMSO) δ 161.81, 158.62, 150.47, 147.50, 134.98, 127.37, 126.88, 126.73, 120.40, 41.42, 27.74, 11.08. HRMS (M+H⁺) calcd. 305.13969, found 305.13984. IR 3202, 2931, 1650, 1612, 1589, 1538, 1473, 1457, 1442, 1413, 1375, 1348, 1270, 1240, 1191, 1178, 1113, 1087, 1019, 972, 923, 903, 878, 839, 810, 798, 764, 730, 691.

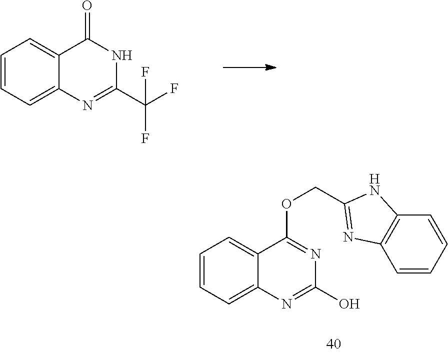 [SMILES: O=c1[nH]c(C(F)(F)F)nc2ccccc12 ; Oc1nc(OCc2nc3ccccc3[nH]2)c2ccccc2n1]

### Preparation of 2-hydroxy-4-(((1H-benzo[d]imidazol-2-yl)methoxy)quinazoline (40)

To a 50 mL RB-flask was added substrate (0.110 g, 0.51 mmol) and 5 mL DMF. Under rapid stirring NaH (60% in mineral oil, 26 mg, 0.64 mmol, 1.25 equiv) was added and allowed to stir for 5 minutes. To this mixture was added 2-chloromethylbenzimidazole (107 mg, 0.64 mmol, 1.25 equiv) and the mixture heated to 80° C. overnight. After allowing the mixture to cool to rt, it was poured into a sep. funnel and diluted with EtOAc (15 ml) and water (10 mL). The phases were separated and the aq. phase extracted with 10 ml EtOAc. The organic phases were combined, washed 3×10 mL water and then 10 mL brine, dried over MgSO₄, filtered and concentrated. Purification by SiO₂ (10 g, slow gradient from 30-100% EtOAc in dichloromethane) afforded an impure mixture. Further purification by SiO₂ (10 g, 0-4% MeOH in dichloromethane) afforded XX as the sole clearly identifiable product as an off-white solid. 17 mg, 9%. ¹H NMR (500 MHz, DMSO-d₆) δ 12.39 (s, 1H), 11.63 (s, 1H), 7.97 (dd, J=7.9, 1.4 Hz, 1H), 7.72 (ddd, J=8.5, 7.3, 1.5 Hz, 1H), 7.51 (d, J=7.6 Hz, 1H), 7.41 (d, J=6.9 Hz, 1H), 7.26 (ddd, J=8.8, 7.8, 1.6 Hz, 2H), 7.13 (tt, J=7.8, 6.3 Hz, 2H), 5.30 (s, 2H). 13C NMR (126 MHz, DMSO) δ 162.49, 151.08, 150.59, 143.51, 140.10, 135.67, 134.63, 127.95, 123.10, 122.22, 121.56, 118.72, 115.74, 114.40, 111.48, 49.06, 38.82. HRMS (M+H⁺) calcd. 291.10330, found 291.10333. IR 3367, 2922, 2852, 1713, 1660, 1622, 1520, 1492, 1455, 1424, 1383, 1348, 1297, 1272, 1194, 1132, 1025, 981, 925, 790, 736, 690, 681.

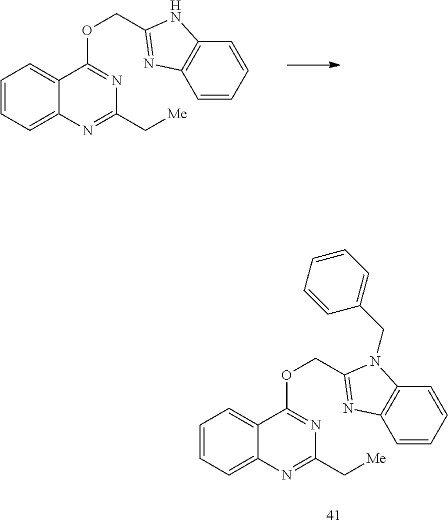 [SMILES: CCc1nc(OCc2nc3ccccc3[nH]2)c2ccccc2n1 ; CCc1nc(OCc2nc3ccccc3n2Cc2ccccc2)c2ccccc2n1]

### Preparation of 2-ethyl-4-((1-benzyl-1H-benzo[d]imidazol-2-yl)methoxy)quinazoline (41)

To a dried 10-mL microwave vial was added substrate (60 mg, 0.19 mmol) and 5 mL DMF. K₂CO₃ (55 mg, 0.4 mmol, 2 equiv) was added as a solid, followed by BnCl (25 mg, 22 μL, 0.19 mmol, 1 equiv). The reaction was allowed to stir overnight at 50° C. The mixture was then extracted with 10 mL each of dichloromethane and water. The aq. phase was washed with a further 10 mL dichloromethane, the combined organic phases were dried over MgSO₄, filtered and concentrated.

Purification by SiO₂ (10 g, 0-70% EtOAc in dichloromethane) to afford the product as a white solid. 42 mg, 54%. ¹H NMR (500 MHz, DMSO-d₆) δ 8.10 (dd, J=8.0, 1.5 Hz, 1H), 7.85 (ddd, J=8.5, 7.1, 1.6 Hz, 1H), 7.68 (dd, J=8.2, 1.1 Hz, 1H), 7.58-7.49 (m, 3H), 7.38 (dd, J=8.1, 6.6 Hz, 2H), 7.34-7.30 (m, 1H), 7.28 (d, J=6.9 Hz, 1H), 7.21 (ddd, J=8.2, 7.2, 1.2 Hz, 1H), 7.15 (ddd, J=8.2, 7.2, 1.2 Hz, 1H), 5.70 (s, 2H), 5.62 (s, 2H), 2.81 (q, J=7.2 Hz, 2H), 1.21 (t, J=7.2 Hz, 3H). 13C NMR (126 MHz, DMSO) δ 161.62, 158.67, 150.42, 147.48, 142.37, 137.03, 136.16, 135.03, 129.30, 128.17, 127.35, 127.28, 126.91, 126.74, 122.91, 122.22, 120.22, 119.38, 110.87, 46.82, 27.71, 11.13. HRMS (M+H⁺) calcd. 395.18664, found 395.18610. IR 2933, 1671, 1588, 1567, 1497, 1464, 1440, 1375, 1340, 1325, 1280, 1245, 1201, 1186, 1142, 1111, 1078, 1025, 1011, 971, 914, 889, 846, 811, 774, 741, 722, 697, 654.

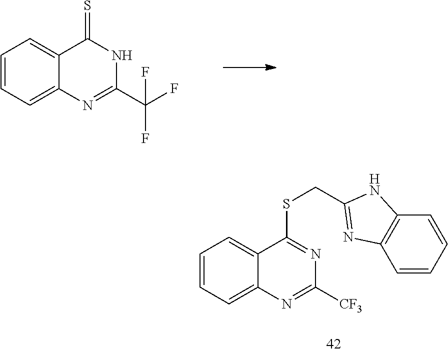 [SMILES: FC(F)(F)c1nc2ccccc2c(=S)[nH]1 ; FC(F)(F)c1nc(SCc2nc3ccccc3[nH]2)c2ccccc2n1]

### Preparation of 2-trifluoromethyl-4-(((1H-benzo[d]imidazol-2-yl)thiomethyl)quinazoline (42)

To a 25 mL RB-flask containing substrate (63 mg, 0.27 mmol) was added MeOH (2 mL), chloromethylbenzimidazole (91 mg, 0.55 mmol, 2 equiv) and sodium methoxide (74 mg, 1.35 mmol, 5 equiv). The mixture was then stirred for 3 h. The pH was adjusted to 7 with sat. aq. NaHCO₃ and then extracted with dichloromethane (3×20 mL). The organic phases were combined, washed with brine (10 mL) dried over MgSO₄, filtered and concentrated. Purification by SiO₂ (10-100% EtOAc in hexanes) afforded the product as a white solid. 74 mg, 75%. ¹H NMR (500 MHz, DMSO-d₆) δ 12.45 (s, 1H), 8.32 (dd, J=8.4, 4.7 Hz, 1H), 8.17 (q, J=4.1 Hz, 2H), 7.95 (dq, J=8.4, 4.3 Hz, 1H), 7.57-7.44 (m, 2H), 7.17 (dd, J=6.1, 3.1 Hz, 2H), 4.97 (s, 2H). ¹³C NMR (126 MHz, DMSO) δ 173.24, 150.42, 150.13, 149.78, 147.12, 136.51, 131.02, 129.60, 124.47, 123.51, 122.31, 121.23, 119.04, 27.73. HRMS (M+H⁺) calcd. 361.07293 found 361.07398. IR 3055, 1567, 1487, 1435, 1390, 1351, 1293, 1274, 1247, 1195, 1137, 1108, 1024, 998, 881, 847, 740, 690.

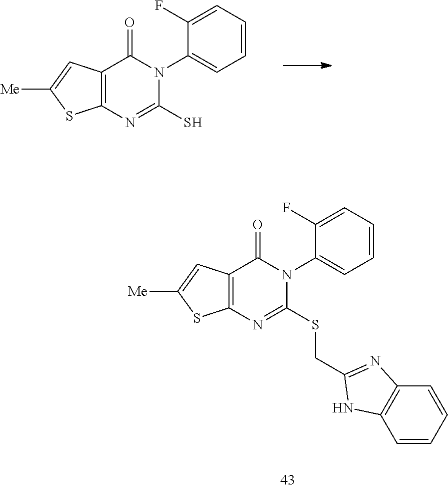 [SMILES: Cc1cc2c(=O)n(-c3ccccc3F)c(S)nc2s1 ; Cc1cc2c(=O)n(-c3ccccc3F)c(SCc3nc4ccccc4[nH]3)nc2s1]

### Preparation of (43)

To a 10-mL microwave vial was added substrate (100 mg, 0.34 mmol), EtOH (1.5 mL) and 2-chloromethylbenzimidazole (69 mg, 0.41 mmol, 1.2 equiv). To this stirring mixture was added 0.5 mL 1N NaOH and the reaction monitored by TLC. After reaction was complete (TLC) the material was poured into a sep. funnel, diluted with water (10 mL) and extracted with EtOAc (2×15 mL). The organic layers were combined, washed with brine (15 mL), dried over MgSO₄, filtered and concentrated. Purification by SiO₂ (10 g, 20% EtOAc in dichloromethane) afforded the product as an off-white solid. 57 mg, 40%. ¹H NMR (300 MHz, DMSO-d₆) δ 12.42 (s, 1H), 7.67 (td, J=7.7, 6.0 Hz, 2H), 7.59-7.36 (m, 4H), 7.13 (m, 3H), 4.65 (s, 2H). 13C NMR (76 MHz, DMSO) δ 162.48, 156.98, 156.58, 149.49, 137.25, 132.00, 126.13, 122.38, 120.99, 119.78, 117.36, 55.38, 31.17. HRMS (M+H⁺) calcd. 423.07441, found 423.07652. IR 2917, 1673, 1594, 1498, 1452, 1434, 1408, 1308, 1264, 1192, 1114, 1086, 1027, 933, 840, 819, 792, 763, 740.

### Example 3

### Analogues of Aryl Hydrocarbon Receptor Ligands for the Prevention and Treatment of Inflammatory Disorders

### 3.1 Overview

The presently disclosed subject matter demonstrates, in part, that activation of the aryl hydrocarbon receptor (AHR) in mice and human tissue can inhibit TLR4 signaling, and thus serve to dampen inflammation in the gut. The finding that AHR activation could inhibit TLR4 signaling led to a search for novel AHR agonists that could be used as therapeutics for NEC. To this end, the AHR analogue “A18”, which had been identified and sold commercially as the proton pump inhibitor “Lansoprazole,” was identified as an AHR agonist. The administration of lansoprazole, i.e., A18, effectively prevented and treated NEC in mouse models, and dampened inflammation in the intestine of human resected at the time of surgery for NEC.

### 3.2 Results and Discussion

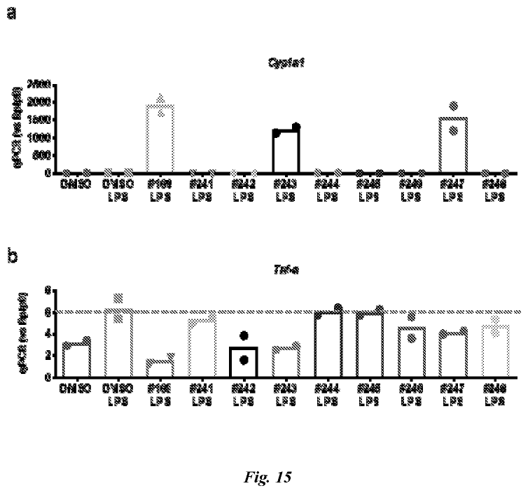

Analogues of A18 were then synthesized and developed. A total of 235 analogues were tested in mouse enteroid cell lines. Compound #169, and other structures, referred to herein as compound #243 and compound #247, activated AHR signaling (FIG. 15a) and significantly inhibited TLR4 signaling (FIG. 15b) as indicated by attenuated Tnf-a induction by TLR4 ligands lipopolysaccharide (LPS) in mouse enteroids.

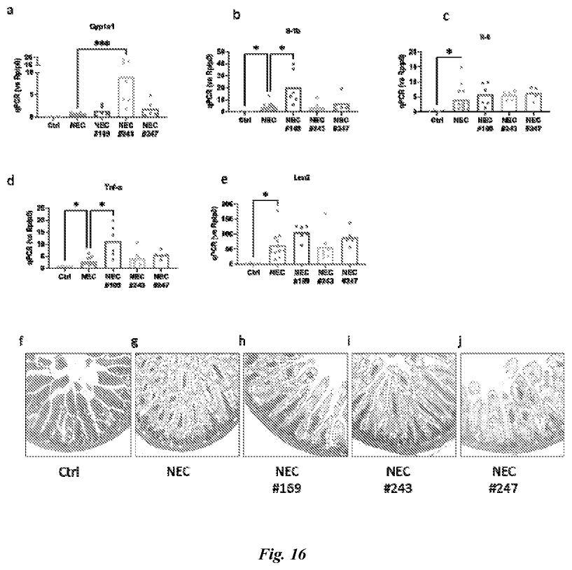

To determine whether compound #169, compound #243, or compound #247 could potentially be administered as a gut protective agent against NEC, compound #169, compound #243, or compound #247 was supplemented into infant formula, which was then administered to pups in a mouse NEC model. As shown in FIG. 16a, administration of 20 mg/kg/day of compound #247 significantly induced the expression of Cyp1a1, a hallmark of AHR activation, while administration of 20 mg/kg/day of compound #169 or compound #243 did not induce the expression of Cyp1a1 in vivo. Administration of compound #169, compound #243, or compound #247, however, did not reduce the severity of NEC, as manifested by similar expression of IL-1b, IL-6, Tnf-a and Lcn2 in the intestinal epithelium (FIG. 16b-FIG. 16e) and reduced histological injury (FIG. 16f-FIG. 16j).

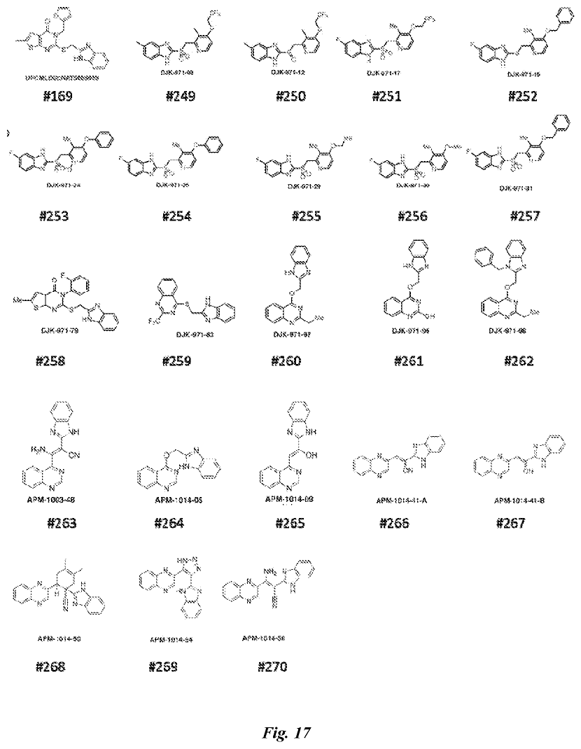

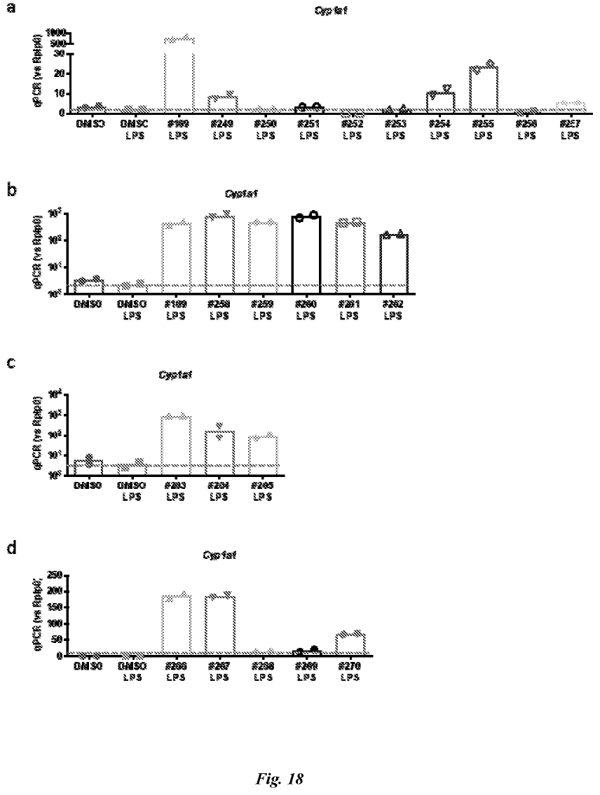

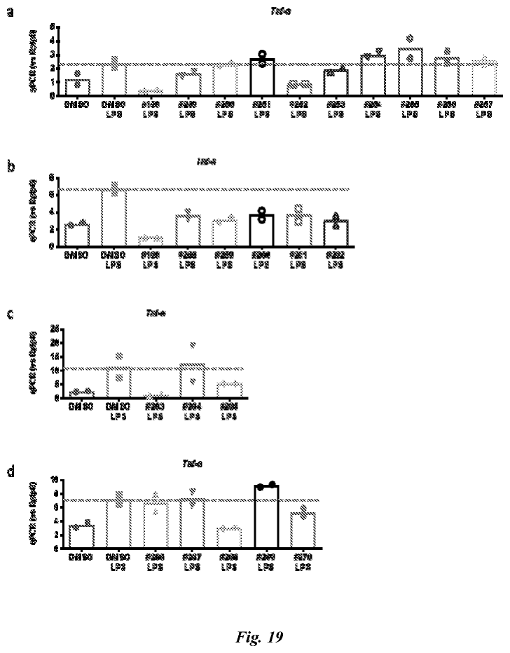

Analogues of compound #169 were then synthesized and developed (FIG. 17). A total of 22 analogues were tested in mouse enteroid cell lines for activation of AHR signaling (FIG. 18) and inhibition of TLR4 signaling (FIG. 19). One representative compound, referred to herein as compound #263, significantly inhibited the TLR4 signaling in mouse enteroids to a similar extent than compound #169 (FIG. 19).

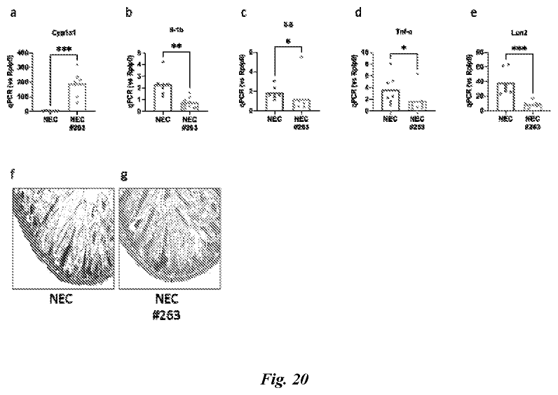

To further determine whether compound #263 could potentially be administered as a gut protective agent against NEC, compound #263 was supplemented into the infant formula, which was then administered to pups in the mouse NEC model. As shown in FIG. 20a, administration of 20 mg/kg/day of compound #263 significantly induced the expression of Cyp1a1. Administration of 20 mg/kg/day of compound #263 also significantly reduced the severity of experimental NEC in the pups, as manifest by reduced expression of IL-1b, IL-6, Tnf-a and Lcn2 in the intestinal epithelium (FIG. 20b-FIG. 20e) and reduced histological injury (FIG. 20f-FIG. 20g).

### 3.3 Methods

### 3.3.1 Mice

The C57BL/6J mice were purchased from the Jackson Laboratory. All mice were housed in a specific pathogen free environment on a 12-hour-light/12-hour-dark cycle with free access to water and standard rodent chow (Teklad global 18% protein rodent diets, Envigo) except otherwise specified. All mouse experiments were approved by the Johns Hopkins University Animal Care and Use Committee.

### 3.3.2 Induction of NEC in Mice

Experimental NEC was induced in 7-day old mice as we have previously described, Good et al., 2015; Sodhi et al., 2018; Egan et al., 2016, by gavage feeding newborn mice with formula containing Similac Advance infant formula (Abbott Nutrition): Esbilac (PetAg) canine milk replacer, 2:1 ratio, which was supplemented with enteric bacteria made from a stock created from a specimen obtained from an infant with surgical NEC five times per day.

Additionally, the mice were subjected to hypoxia (5% 02-95% N₂) for 10 min in a hypoxia chamber (Billups-Rothenberg) twice daily for 4 days. The compound #169, compound #243, compound #247 or compound #263 administered by oral gavage during the induction of NEC at the dose of 20 mg/kg body weight/day.

Age-matched breast milk-fed mouse pups were used as healthy controls. Evaluation of ileal histology by H&E staining and expression of pro-inflammatory cytokines by qRT-PCR at a fixed point in the terminal ileum 2-cm proximal to the cecum, were used to determine the disease severity.

3.3.3 Harvest and Culture of Enteroids from Mouse Ileum

Primary intestinal crypt cultures (enteroids) were generated from the ileum of neonatal (p7-p11) mice at as described, Neal et al., 2012, and maintained in Matrigel (Corning). The enteroids were digested and passed using TrypLE Express (Gibco) weekly, and used between passage 3 and 10 for all experiments. The enteroids were pre-treated with tested compound (40 uM, overnight) and then treated with LPS (50 μg/mL) for 6 hours for further analysis.

3.3.4 RNA Isolation, cDNA Synthesis, Quantification of mRNA and miRNA.

Total RNA was isolated using the RNeasy mini kit (Qiagen) and complementary DNA was synthesized from 0.5 μg RNA using QuantiTect Reverse Transcription kit (Qiagen) following the manufacturer's protocols. The mRNA quantification was performed on the Bio-Rad CFX96 Real-Time System (Bio-Rad) using iTaq™ universal SYBR® Green supermix (Bio-Rad) and the primers listed in Table 2. The relative mRNA expression levels were normalized against the expression of the housekeeping gene ribosomal protein lateral stalk subunit P0 (Rplp0).

TABLE 2
Primers.

| | | | Amplicon |
|---|---|---|---|
| Gene | Forward primer | Reverse primer | size (bp) |
| Cyp1a1 | TTTGTGAACCAGTGGCAGGT | TCTCCGATGCACTTTCGCTT | 158 |
| Il-1b | AGTGTGGATCCCAAGCAATACCCA | TGTCCTGACCACTGTTGTTTCCCA | 175 |
| Il-6 | CCAATTTCCAATGCTCTCCT | ACCACAGTGAGGAATGTCCA | 182 |
| Lcn2 | ACAACCAGTTCGCCATGGTAT | AAGCGGGTGAAACGTTCCTT | 121 |
| Rplp0 | GGCGACCTGGAAGTCCAACT | CCATCAGCACCACAGCCTTC | 143 |
| Tnf-α | TTCCGAATTCACTGGAGCCTCGAA | TGCACCTCAGGGAAGAATCTGGAA | 144 |

### REFERENCES

All publications, patent applications, patents, and other references mentioned in the specification are indicative of the level of those skilled in the art to which the presently disclosed subject matter pertains. All publications, patent applications, patents, and other references are herein incorporated by reference to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents forms part of the common general knowledge in the art.
Neu, J. & Walker, W. A. Necrotizing enterocolitis. New England Journal of Medicine (2011). doi:10.1056/NEJMra1005408
Hackam, D. J., Sodhi, C. P. & Good, M. New insights into necrotizing enterocolitis: From laboratory observation to personalized prevention and treatment. Journal of Pediatric Surgery (2019). doi:10.1016/j.jpedsurg.2018.06.012
Flahive, C., Schlegel, A. & Mezoff, E. A. Necrotizing Enterocolitis: Updates on Morbidity and Mortality Outcomes. Journal of Pediatrics (2020). doi:10.1016/j.jpeds.2019.12.035
Leaphart, C. L. et al. A critical role for TLR4 in the pathogenesis of necrotizing enterocolitis by modulating intestinal injury and repair. J Immunol 179, 4808-4820 (2007).
Sodhi, C. P. et al. Intestinal epithelial toll-like receptor 4 regulates goblet cell development and is required for necrotizing enterocolitis in mice. Gastroenterology 143, 705-708 (2012).
Werts, A. D. et al. A Novel Role for Necroptosis in the Pathogenesis of Necrotizing Enterocolitis. Cell. Mol. Gastroenterol. Hepatol. (2019). doi:10.1016/j.jcmgh.2019.11.002
Jilling, T. et al. The Roles of Bacteria and TLR4 in Rat and Murine Models of Necrotizing Enterocolitis. J. Immunol. (2006). doi:10.4049/jimmuno1.177.5.3273
Fawley, J. et al. Single-Immunoglobulin Interleukin-1-Related Receptor regulates vulnerability to TLR4-mediated necrotizing enterocolitis in a mouse model. Pediatr Res 83, 164-174 (2017).
Gribar, S. C. et al. Reciprocal Expression and Signaling of TLR4 and TLR9 in the Pathogenesis and Treatment of Necrotizing Enterocolitis. J Immunol. 182, 636-646 (2014).
Afrazi, A. et al. Toll-like receptor 4-mediated endoplasmic reticulum stress in intestinal crypts induces necrotizing enterocolitis. J. Biol. Chem. 289, 9584-9599 (2014).
Hackam, D. J. & Sodhi, C. P. Toll-Like Receptor—Mediated Intestinal Inflammatory Imbalance in the Pathogenesis of Necrotizing Enterocolitis. CMGH (2018). doi:10.1016/j.jcmgh.2018.04.001
Jin, Y.-T., Duan, Y., Deng, X.-K. & Lin, J. Prevention of necrotizing enterocolitis in premature infants—an updated review. World J Clin. Pediatr. (2019). doi:10.5409/wjcp.v8.i2.23
Underwood, M. A. Probiotics and the prevention of necrotizing enterocolitis. J Pediatr Surg (2018). doi:10.1016/j.jpedsurg.2018.08.055
Ogunyemi, D., Murillo, M., Jackson, U., Hunter, N. & Alperson, B. The relationship between placental histopathology findings and perinatal outcome in preterm infants. J. Matern. Neonatal Med. (2003). doi:10.1080/jmf.13.2.102.109
Duci, M. et al. Maternal and placental risk factors associated with the development of necrotizing enterocolitis (NEC) and its severity. J. Pediatr. Surg. (2019). doi:10.1016/j.jpedsurg.2019.04.018
Englund-Ogge, L. et al. Maternal dietary patterns and preterm delivery: Results from large prospective cohort study. BMJ (2014). doi:10.1136/bmj.g1446
Saunders, L. et al. Effect of a Mediterranean diet during pregnancy on fetal growth and preterm delivery: Results from a French Caribbean mother-child cohort study (TIMOUN). Paediatr. Perinat. Epidemiol. (2014). doi:10.1111/ppe.12113
Rothhammer, V. & Quintana, F. J. The aryl hydrocarbon receptor: an environmental sensor integrating immune responses in health and disease. Nature Reviews Immunology (2019). doi:10.1038/s41577-019-0125-8
Amakura, Y. et al. Influence of food polyphenols on aryl hydrocarbon receptor-signaling pathway estimated by in vitro bioassay. Phytochemistry (2008). doi:10.1016/j.phytochem.2007.07.022
Ahlfors, H. et al. IL-22 Fate Reporter Reveals Origin and Control of IL-22 Production in Homeostasis and Infection. J. Immunol. 193, 4602-4613 (2014).
Chong, C. R., Chen, X., Shi, L., Liu, J. O. & Sullivan, D. J. A clinical drug library screen identifies astemizole as an antimalarial agent. Nat. Chem. Biol. (2006). doi:10.1038/nchembio806
Good, M. et al. Breast milk protects against the development of necrotizing enterocolitis through inhibition of Toll-like receptor 4 in the intestinal epithelium via activation of the epidermal growth factor receptor. Mucosal Immunol. (2015). doi:10.1038/mi.2015.30
Sodhi, C. P. et al. Fat composition in infant formula contributes to the severity of necrotizing enterocolitis. Br. J. Nutr. (2018). doi:10.1017/S₀₀₀₇₁₁₄₅₁₈₀₀₁₈₃₆
Egan, C. E. et al. Toll-like receptor 4-mediated lymphocyte influx induces neonatal necrotizing enterocolitis. J Clin. Invest. 126, 495-508 (2016).
Good, M. et al. Lactobacillus rhamnosus HN001 decreases the severity of necrotizing enterocolitis in neonatal mice and preterm piglets: evidence in mice for a role of TLR9. Am J Physiol Gastrointest Liver Physiol 306, G1021-32 (2014).
Sun et al., Human milk fortification affects gut microbiota in preterm pigs. The Journal of Nutrition, (2018) doi.org/10.1093/jn/nxx033
Brawner, K. M. et al. Depletion of dietary aryl hydrocarbon receptor ligands alters microbiota composition and function. Sci. Rep. (2019). doi:10.1038/s41598-019-51194-w
Neal, M. D. et al. Toll-like receptor 4 is expressed on intestinal stem cells and regulates their proliferation and apoptosis via the p53 up-regulated modulator of apoptosis. J. Biol. Chem. 287, 37296-37308 (2012).
Hepworth, M. R. et al. Innate lymphoid cells regulate CD4+T-cell responses to intestinal commensal bacteria. Nature (2013). doi:10.1038/nature12240
Lu, P., Sodhi, C. P. & Hackam, D. J. Toll-like receptor regulation of intestinal development and inflammation in the pathogenesis of necrotizing enterocolitis. Pathophysiology 21, 81-93 (2014).
Kiss, E. A. et al. Natural aryl hydrocarbon receptor ligands control organogenesis of intestinal lymphoid follicles. Science (80-.). (2011). doi:10.1126/science.1214914
Schiering, C. et al. Feedback control of AHR signaling regulates intestinal immunity. Nature (2017). doi:10.1038/nature21080
Chen, L. et al. IL-23 activates innate lymphoid cells to promote neonatal intestinal pathology. Mucosal Immunol. (2015). doi:10.1038/mi.2014.77
Singh, R. et al. Enhancement of the gut barrier integrity by a microbial metabolite through the Nrf2 pathway. Nat. Commun. (2019). doi:10.1038/s41467-018-07859-7
He, X., Jing, Z. & Cheng, G. MicroRNAs: New regulators of toll-like receptor signaling pathways. BioMed Research International (2014). doi:10.1155/2014/945169
Han, S. M. et al. Trends in incidence and outcomes of necrotizing enterocolitis over the last 12 years: A multicenter cohort analysis. J. Pediatr. Surg. (2020). doi:10.1016/j.jpedsurg.2020.02.046
Nino, D. F., Sodhi, C. P. & Hackam, D. J. Necrotizing enterocolitis: new insights into pathogenesis and mechanisms. Nat Rev Gastroenterol Hepatol 13, 590-600 (2016).
Gao, J. et al. Impact of the gut microbiota on intestinal immunity mediated by tryptophan metabolism. Frontiers in Cellular and Infection Microbiology (2018). doi:10.3389/fcimb.2018.00013
Shinde, R. and McGaha, T. L. The Aryl Hydrocarbon Receptor: Connecting Immunity to the Microenvironment. Trends Immunol. 39, 1005-1020 (2018).
Veldhoen, M. et al. The aryl hydrocarbon receptor links TH17-cell-mediated autoimmunity to environmental toxins. Nature (2008). doi:10.1038/nature06881
Monteleone, I. et al. Aryl hydrocarbon receptor-induced signals up-regulate IL-22 production and inhibit inflammation in the gastrointestinal tract. Gastroenterology (2011). doi:10.1053/j.gastro.2011.04.007
Alvarado, D. M. et al. Epithelial Indoleamine 2,3-Dioxygenase 1 Modulates Aryl Hydrocarbon Receptor and Notch Signaling to Increase Differentiation of Secretory Cells and Alter Mucus-Associated Microbiota. Gastroenterology (2019). doi:10.1053/j.gastro.2019.07.013

Wu, Y. Y., Hsu, C. M., Chen, P. H., Fung, C. P. & Chen, L. W. Toll-like receptor stimulation induces nondefensin protein expression and reverses antibiotic-induced gut defense impairment. Infect. Immun. (2014). doi:10.1128/IAI.01578-14
Yang, Z.-C., Yu, F., Wang, Y.-Q. & Wei, J.-F. Stereoselective Pharmacodynamics and Pharmacokinetics of Proton Pump Inhibitors. Curr. Drug Metab. (2016). doi:10.2174/1389200217666160322143857
Jin, U. H., Lee, S. O., Pfent, C. & Safe, S. The aryl hydrocarbon receptor ligand omeprazole inhibits breast cancer cell invasion and metastasis. BMC Cancer (2014). doi:10.1186/1471-2407-14-498
Gutierrez-Vazquez, C. & Quintana, F. J. Regulation of the Immune Response by the Aryl Hydrocarbon Receptor. Immunity (2018). doi:10.1016/j.immuni.2017.12.012
Denison, M. S. & Faber, S. C. And now for something completely different: Diversity in ligand-dependent activation of Ah receptor responses. Current Opinion in Toxicology (2017). doi:10.1016/j.cotox.2017.01.006
Landes, B. D., Petite, J. P. & Flouvat, B. Clinical Pharmacokinetics of Lansoprazole. Clinical Pharmacokinetics (1995). doi:10.2165/00003088-199528060-00004
Alganabi, M., Lee, C., Bindi, E., Li, B. & Pierro, A. Recent advances in understanding necrotizing enterocolitis. F1000Research (2019). doi:10.12688/f1000research.17228.1
Parlapani, E. et al. The Mediterranean diet adherence by pregnant women delivering prematurely: association with size at birth and complications of prematurity. J. Matern. Neonatal Med. (2019). doi:10.1080/14767058.2017.1399120
Luniwal, A.; Wang, L.; Pavlovsky, A.; Erhardt, P. W.; Viola, R. E. Bioorganic & Medicinal Chemistry 2012, 20, 2950-2956.
Soler, M.; Figueras, E.; Serrano-Plana, J.; González-Bártulos, M.; Massaguer, A.; Company, A.; Martinez, M. A.; Malina, J.; Brabec, V.; Feliu, L.; Planas, M.; Ribas, X.; Costas, M. Inorganic Chemistry 2015, 54, 10542-10558.
Chen, D.; Zhou, Y.; Zhou, H.; Liu, S.; Liu, Q.; Zhang, K.; Uozumi, Y. Synlett 2018, 29, 1765-1768.
Liu, X.; Zhang, S.-B.; Dong, Z.-B. European Journal of Organic Chemistry 2018, 2018, 5406-5411.
Mavrova, A. T.; Yancheva, D.; Anastassova, N.; Anichina, K.; Zvezdanovic, J.; Djordjevic, A.; Markovic, D.; Smelcerovic, A. Bioorganic & Medicinal Chemistry 2015, 23, 6317-6326.
Zhao, W.; Wang, X.; Yang, C. Synthetic Communications 2014, 44, 150-160.
Limnios, D.; Kokotos, C. G. Chemistry—A European Journal 2014, 20, 559-563.
Schießl, J.; Stein, P. M.; Stirn, J.; Emler, K.; Rudolph, M.; Rominger, F.; Hashmi, A. S. K. Advanced Synthesis & Catalysis 2019, 361, 725-738.
Duan, X.-F.; Ma, Z.-Q.; Zhang, F.; Zhang, Z.-B. The Journal of Organic Chemistry 2009, 74, 939-942.
Palav, A.; Misal, B.; Emolla, A.; Parab, V.; Waske, P.; Khandekar, D.; Chaudhary, V.; Chaturbhuj, G. Organic Process Research & Development 2019, 23, 244-251.
Li, G.; Yang, S.; Lv, B.; Han, Q.; Ma, X.; Sun, K.; Wang, Z.; Zhao, F.; Lv, Y.; Wu, H. Organic & Biomolecular Chemistry 2015, 13, 11184-11188.
Cignarella, G.; Vianello, P.; Berti, F.; Rossoni, G. European Journal of Medicinal Chemistry 1996, 31, 359-364.
Zhang, F.; Duan, X.-F. Organic Letters 2011, 13, 6102-6105.
Kubo, K.; Oda, K.; Kaneko, T.; Satoh, H.; Nohara, A. CHEMICAL & PHARMACEUTICAL BULLETIN 1990, 38, 2853-2858.
International PCT Patent Application Publication No. WO2014/154612 for Novel Pyridine Derivatives, to Gavelle et al., published Oct. 2, 2014.
International PCT Patent Application Publication No. WO2010/117425 for Certain Substituted Pyrimidines, Pharmaceutical Compositions Thereof, and Methods for Their Use, to Shi et al., published Oct. 14, 2010.
Kuehler, T. C.; Fryklund, J.; Bergman, N.-k.; Weilitz, J.; Lee, A.; Larsson, H. Journal of Medicinal Chemistry 1995, 38, 4906-4916.
Kühler, T. C.; Swanson, M.; Shcherbuchin, V.; Larsson, H.; Mellgârd, B.; Sjöström, J.-E. Journal of Medicinal Chemistry 1998, 41, 1777-1788.
Weidmann, K.; Herling, A. W.; Lang, H. J.; Scheunemann, K. H.; Rippel, R.; Nimmesgem, H.; Scholl, T.; Bickel, M.; Metzger, H. Journal of Medicinal Chemistry 1992, 35, 438-450.
International PCT Patent Application Publication No. WO2001034578 for Compounds with Anti-Helicobacter Pylori Activity, to Carcanague et al., published May 17, 2001.
Abdullaha, M.; Mohammed, S.; Ali, M.; Kumar, A.; Vishwakarma, R. A.; Bharate, S. B. The Journal of Organic Chemistry 2019, 84, 5129-5140.
Alexandre, F.-R.; Berecibar, A.; Wrigglesworth, R.; Besson, T. Tetrahedron 2003, 59, 1413-1419.
Dayakar, C.; Jyothi, D.; Suman, P.; Raju, B. C. Synthetic Communications 2015, 45, 1642-1651.

Although the foregoing subject matter has been described in some detail by way of illustration and example for purposes of clarity of understanding, it will be understood by those skilled in the art that certain changes and modifications can be practiced within the scope of the appended claims.

That which is claimed:

1. An aryl hydrocarbon receptor (AUR) agonist comprising a compound of formula (I): 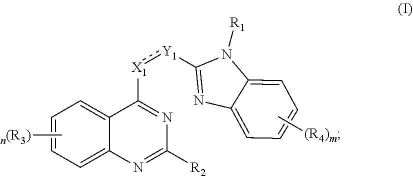
wherein: [symbol]
indicates a single or a double bond;
n and m are each independently selected from the group consisting of 0, 1, 2, 3, and 4;
X₁ is selected from the group consisting of S, O, S(═O), S(═O)₂, and —C(Rₓ)—;
Y₁ is —CH₂— or —C(R_{y})—;
wherein Rₓ and R_{y} are each independently selected from the group consisting of H, hydroxyl, cyano, and amino, or Rₓ and R_{y} together form part of a 5- to 6-membered substituted or unsubstituted cycloalkyl, cycloalkenyl, cycloheteroalkyl, cycloheteroalkenyl, aryl, or heteroaryl;
R₁ is selected from the group consisting of H, substituted or unsubstituted straightchain or branched C₁-C₆ alkyl, and —(CH₂)ₚ—Ar, wherein Ar is selected from the group consisting of substituted aryl, unsubstituted aryl, substituted heteroaryl, and unsubstituted heteroaryl, p is an integer selected from the group consisting of 0, 1, 2, 3, and 4;
R₂ is selected from the group consisting of H, substituted or unsubstituted straightchain or branched C₁-C₆ alkyl, hydroxyl, C₁-C₆ alkoxyl, halogen, —CF₃, cyano, amino, carboxyl, nitro, and mercapto;
each R₃ and R₄ are independently selected from the group consisting of H, substituted or unsubstituted straightchain or branched C₁-C₆ alkyl, hydroxyl, C₁-C₆ alkoxyl, halogen, —CF₃, cyano, amino, carboxyl, nitro, and mercapto; and
pharmaceutically acceptable salts thereof.

2. The compound of claim 1, wherein X₁ is S or O.

3. The compound of claim 1 or claim 2, wherein R₁ is selected from the group consisting of H, substituted or unsubstituted straightchain or branched C₁-C₆ alkyl, and —(CH₂)ₚ—Ar, wherein Ar is substituted aryl or unsubstituted aryl, and p is 1.

4. The compound of any one of claims 1-3, wherein R₂ is selected from the group consisting of H, substituted or unsubstituted straightchain or branched C₁-C₆ alkyl, hydroxyl, and —CF₃.

5. The compound of any one of claims 1-4, wherein the compound of formula (I) is selected from the group consisting of:

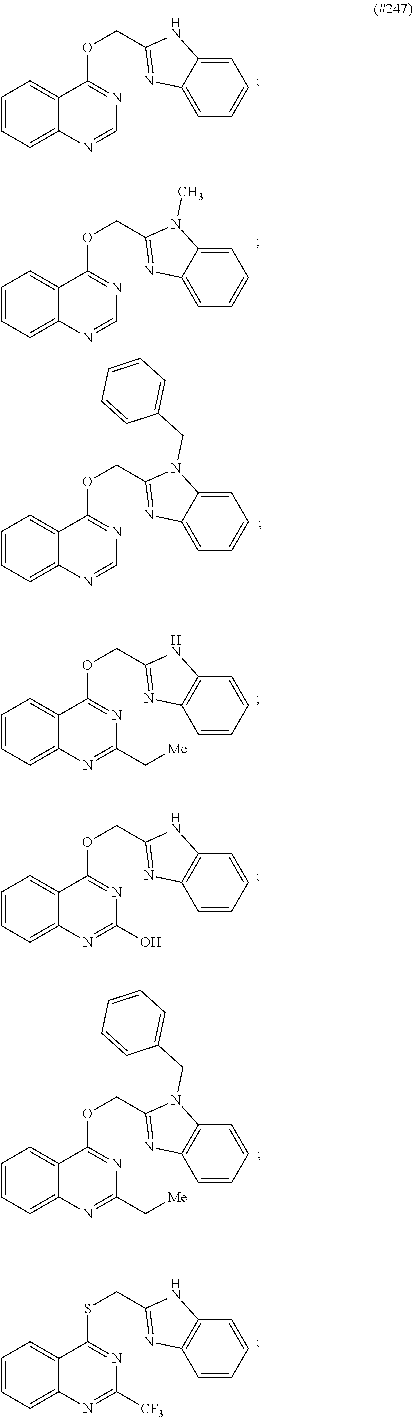

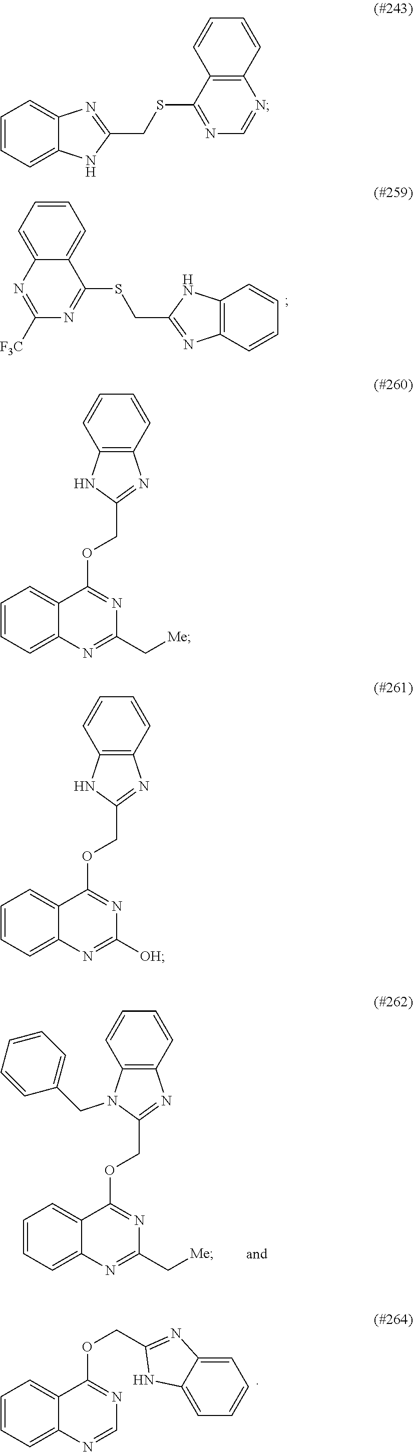

6. The compound of claim 1, wherein X₁ is —C(Rₓ)— and Y₁ is —C(R_{y})—.

7. The compound of claim 6, wherein the compound of formula (I) is selected from the group consisting of:

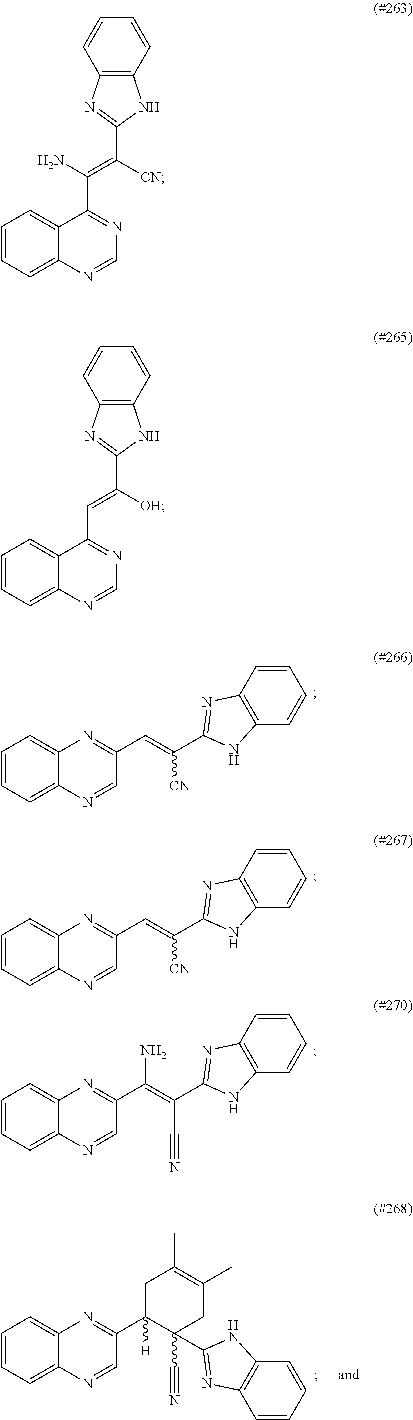 [SMILES: N#C/C(=C(/N)c1ncnc2ccccc12)c1nc2ccccc2[nH]1 ; O/C(=C\c1ncnc2ccccc12)c1nc2ccccc2[nH]1 ; N#CC(=Cc1cnc2ccccc2n1)c1nc2ccccc2[nH]1 ; N#CC(=Cc1cnc2ccccc2n1)c1nc2ccccc2[nH]1 ; N#C/C(=C(/N)c1cnc2ccccc2n1)c1nc2ccccc2[nH]1 ; CC1=C(C)CC(C#N)(c2nc3ccccc3[nH]2)C(c2cnc3ccccc3n2)C1]

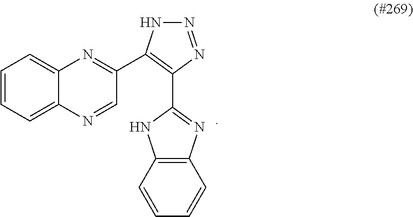 [SMILES: c1ccc2nc(-c3[nH]nnc3-c3nc4ccccc4[nH]3)cnc2c1]

8. An aryl hydrocarbon receptor agonist comprising a compound of formula (II): 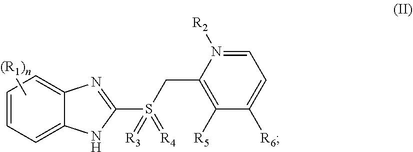
wherein:
n is an integer selected from the group consisting of 0, 1, 2, 3, and 4;
each R₁ is independently halogen or substituted or unsubstituted straightchain or branched C₁-C₆ alkyl;
R₂ is present or absent and when present is oxygen;
R₃ and R₄ are each independently present or absent and when present are oxygen;
R₅ is substituted or unsubstituted straightchain or branched C₁-C₆ alkyl;
R₆ is —O—(CH₂)ₘ-R₇, wherein R₇ is selected from the group consisting of substituted or unsubstituted straightchain or branched C₁-C₆ alkyl, —CF₃, and substituted or unsubstituted aryl; and
pharmaceutically acceptable salts thereof.

9. The compound of claim 8, wherein the compound of formula (II) is:

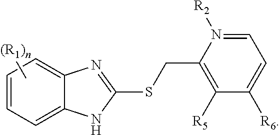

10. The compound of claim 9, wherein the compound of formula (II) is:

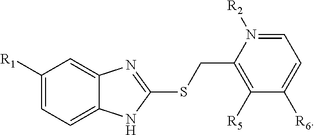

11. The compound of claim 10, wherein the compound of formula (II) is:

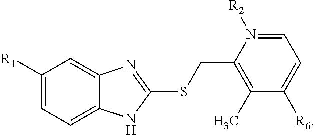

12. The compound of claim 11, wherein the compound of formula (II) is selected from the group consisting of:

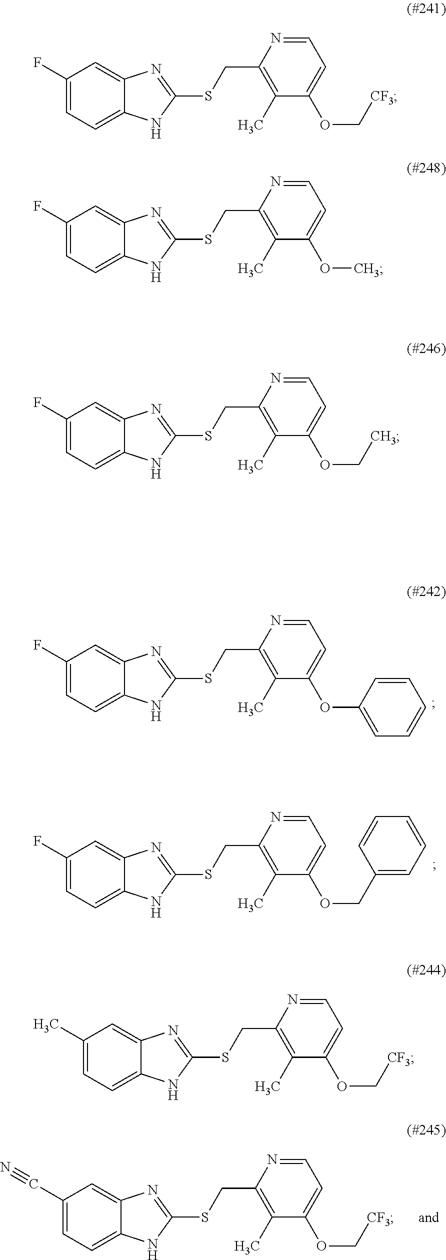

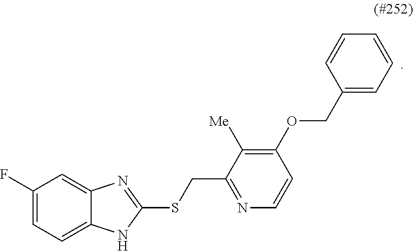 [SMILES: Cc1c(OCc2ccccc2)ccnc1CSc1nc2cc(F)ccc2[nH]1]

13. The compound of claim 8, wherein the compound of formula (II) is:

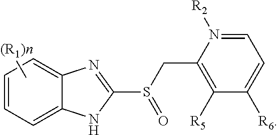

14. The compound of claim 13, wherein the compound of formula (II) is:

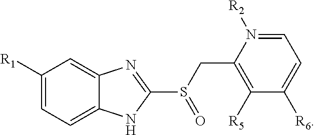

15. The compound of claim 14, wherein the compound of formula (II) is:

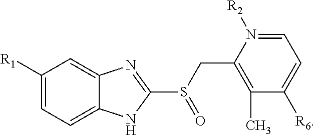

16. The compound of claim 15, wherein the compound of formula (II) is:

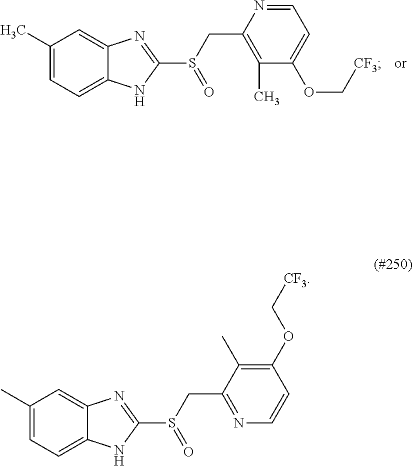 [SMILES: Cc1ccc2[nH]c(S(=O)Cc3nccc(OCC(F)(F)F)c3C)nc2c1 ; Cc1ccc2[nH]c(S(=O)Cc3nccc(OCC(F)(F)F)c3C)nc2c1]

17. The compound of claim 8, wherein the compound of formula (II) is:

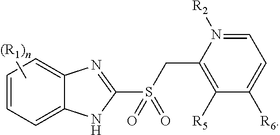

18. The compound of claim 17, wherein the compound of formula (II) is:

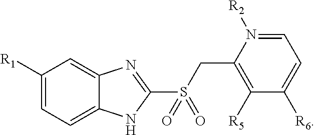

19. The compound of claim 18, wherein the compound of formula (II) is:

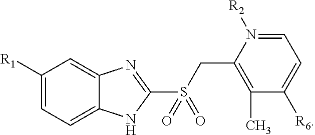

20. The compound of claim 19, wherein the compound of formula (II) is selected from the group consisting of:

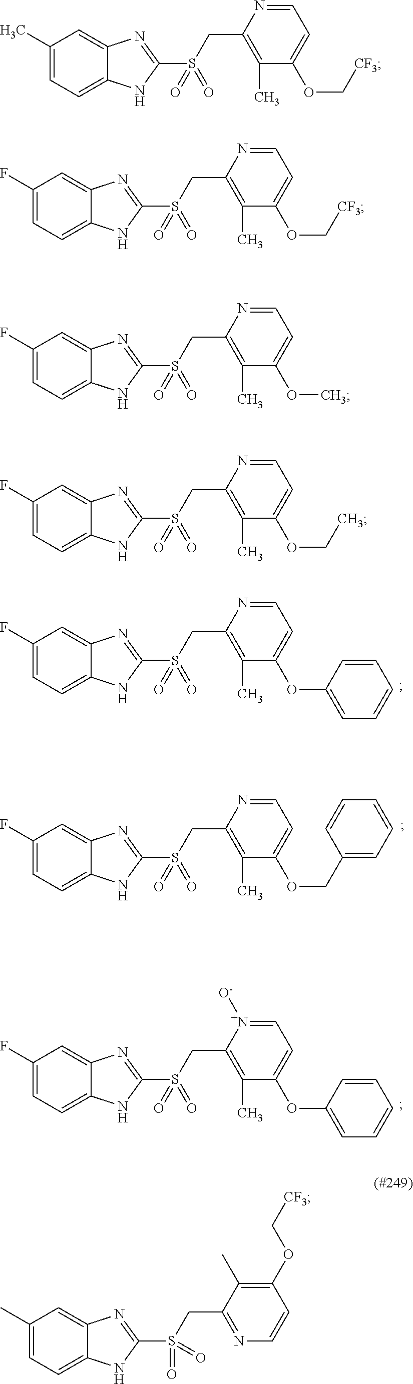

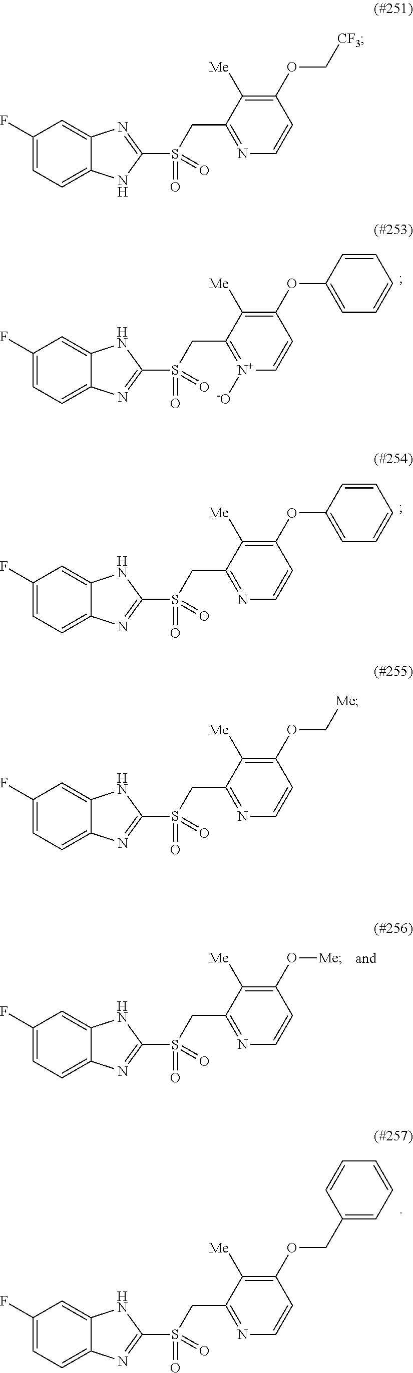

21. An aryl hydrocarbon receptor (AHR) comprising a compound of formula (III): 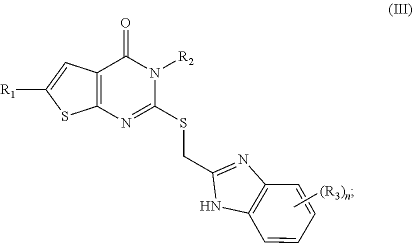
wherein:
n is an integer selected from the group consisting of 0, 1, 2, 3, and 4;
R₁ is substituted or unsubstituted straightchain or branched C₁-C₆ alkyl;
R₂ is selected from the group consisting of substituted aryl, unsubstituted aryl, substituted heteroaryl, and unsubstituted heteroaryal;
each R₃ is independently selected from the group consisting of H, substituted or unsubstituted straightchain or branched C₁-C₆ alkyl, hydroxyl, C₁-C₆ alkoxyl, halogen, —CF₃, cyano, amino, carboxyl, nitro, and mercapto; and
pharmaceutically acceptable salts thereof.

22. The compound of claim 21, wherein the compound of formula (III) is: 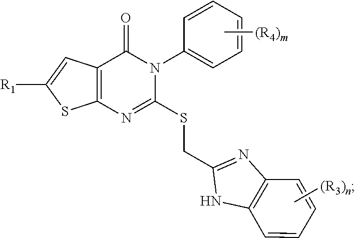
wherein:
m is an integer selected from the group consisting of 0, 1, 2, 3, 4, and 5; and
R₄ is selected from the group consisting of H, substituted or unsubstituted straightchain or branched C₁-C₆ alkyl, hydroxyl, C₁-C₆ alkoxyl, halogen, —CF₃, cyano, amino, carboxyl, nitro, and mercapto.

23. The compound of claim 22, wherein the compound of formula (III) is:

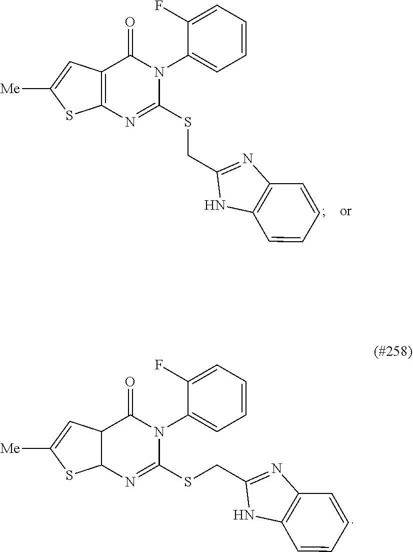 [SMILES: Cc1cc2c(=O)n(-c3ccccc3F)c(SCc3nc4ccccc4[nH]3)nc2s1 ; CC1=CC2C(=O)N(c3ccccc3F)C(SCc3nc4ccccc4[nH]3)=NC2S1]

24. A method for treating or preventing or reducing the risk of an inflammatory disorder associated with a reduced expression of an aryl hydrocarbon receptor (AHR) in a subject in need of treatment thereof, the method comprising administering to the subject an aryl hydrocarbon receptor (AHR) agonist of any one of claims 1-23, a compound having the following structure: 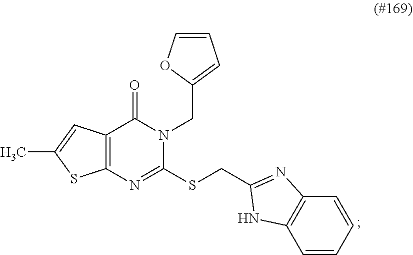 [SMILES: Cc1cc2c(=O)n(Cc3ccco3)c(SCc3nc4ccccc4[nH]3)nc2s1]
or pharmaceutically acceptable salts thereof, to activate the AHR, thereby treating or preventing or reducing the risk of the inflammatory disorder.

25. The method of claim 24, wherein the inflammatory disorder is necrotizing enterocolitis.

26. The method of claim 24, wherein the subject is a human subject.

27. The method of claim 26, wherein the human subject is an infant.

28. The method of claim 27, wherein the human subject is a premature infant.

29. The method of claim 24, wherein the administration is enteral administration.

30. The method of claim 29, wherein the enteral administration is oral administration or gastric administration.

31. An infant nutritional formula comprising a therapeutically effective amount of one or more aryl hydrocarbon receptor (AHR) agonists of any one of claims 1-23, a compound having the following structure: 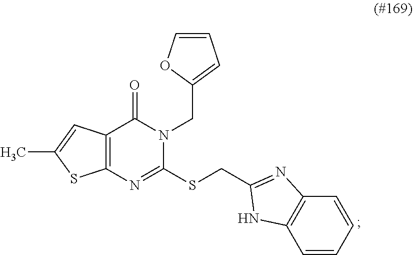 [SMILES: Cc1cc2c(=O)n(Cc3ccco3)c(SCc3nc4ccccc4[nH]3)nc2s1]
or pharmaceutically acceptable salts thereof.

32. The infant nutritional formula of claim 31, wherein the formula is nutritionally complete.

33. The infant nutritional formula of claim 32, wherein the formula comprises one or more ingredients selected from the group consisting of protein, fat, linoleic acid, vitamin A, vitamin C, vitamin D, vitamin E, vitamin K, thiamin (B1), riboflavin (B2), B6, B12, niacin, folic acid, pantothenic acid, calcium, magnesium, iron, zinc, manganese, copper, phosphorous, iodine, sodium chloride, potassium chloride, one or more carbohydrates, including lactose, sucrose, glucose, dextrins, natural and modified starches, nucleotides, emulsifiers, stabilizers, and diluents.

34. The infant nutritional formula of claim 32, wherein the formula is in the form of a liquid, a powder, a capsule, a tablet, or an orally disintegrating tablet.

35. The infant nutritional formula of claim 34, wherein the liquid is in the form of a solution, an emulsion, or a suspension.

36. The infant nutritional formula of claim 32, wherein the formula is adapted for enteral administration to an infant.

37. The infant nutritional formula of claim 36, wherein the enteral administration is oral administration or gastric administration.

38. A method for preventing, reducing the risk of, or reducing the severity of an inflammatory disorder associated with a reduced expression of an aryl hydrocarbon receptor (AHR) in a subject in need of treatment thereof, the method comprising administering to a mother while pregnant with the subject one or more aryl hydrocarbon receptor (AHR) agonist of any one of claims 1-23, a compound having the following structure: 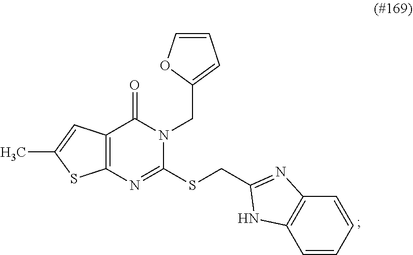 [SMILES: Cc1cc2c(=O)n(Cc3ccco3)c(SCc3nc4ccccc4[nH]3)nc2s1]
or pharmaceutically acceptable salts thereof, to activate the AHR, thereby treating or preventing, reducing the risk of, or reducing the severity of the inflammatory disorder.

39. The method of claim 38, wherein the mother is at risk for delivering the subject prematurely.
